SEQUENCE LISTING (290 sequences)

SEQ ID NO: 1 - DNA, 25 bp, Artificial Sequence
  REGX3.1 Primer
ggagagtaaagttcttgaacttcct

SEQ ID NO: 2 - DNA, 18 bp, Artificial Sequence
  REGX3.1 Primer
agttacgtgccagatcag

SEQ ID NO: 3 - DNA, 24 bp, Artificial Sequence
  REGX3.1 Primer
tgcggcaatagtgtttataacact

SEQ ID NO: 4 - DNA, 23 bp, Artificial Sequence
  REGX3.1 Primer
atgaaagttcaatcattctgtct

SEQ ID NO: 5 - DNA, 18 bp, Artificial Sequence
  REGX3.1 Primer
cggcgtaaaacacgtcta

SEQ ID NO: 6 - DNA, 23 bp, Artificial Sequence
  REGX3.1 Primer
gctaaaaagcacaaatagaagtc

SEQ ID NO: 7 - DNA, 25 bp, Artificial Sequence
  REGX3.1 Primer
tgtctgatgaacagtttaggtgaaa

SEQ ID NO: 8 - DNA, 20 bp, Artificial Sequence
  REGX3.1 Primer
ttgcttcacactcaaaagaa

SEQ ID NO: 9 - DNA, 43 bp, Artificial Sequence
  REGX3.1 Primer
ggagagtaaagttcttgaacttcctagttacgtgccagatcag

SEQ ID NO: 10 - DNA, 47 bp, Artificial Sequence
  REGX3.1 Primer
tgcggcaatagtgtttataacactatgaaagttcaatcattctgtct

SEQ ID NO: 11 - DNA, 24 bp, Artificial Sequence
  REGX1.1 Primer
ttccgtgtaccaagcaatttcatg

SEQ ID NO: 12 - DNA, 19 bp, Artificial Sequence
  REGX1.1 Primer
tgacactaagaggggtgta

SEQ ID NO: 13 - DNA, 23 bp, Artificial Sequence
  REGX1.1 Primer
aagagctatgaattgcagacacc

SEQ ID NO: 14 - DNA, 19 bp, Artificial Sequence
  REGX1.1 Primer
tggacattccccattgaag

SEQ ID NO: 15 - DNA, 18 bp, Artificial Sequence
  REGX1.1 Primer
gtccgaacaactggactt

SEQ ID NO: 16 - DNA, 25 bp, Artificial Sequence
  REGX1.1 Primer
gtcttgattatggaatttaagggaa

SEQ ID NO: 17 - DNA, 21 bp, Artificial Sequence
  REGX1.1 Primer
ctcatgttcacggcagcagta

SEQ ID NO: 18 - DNA, 21 bp, Artificial Sequence
  REGX1.1 Primer
attggcaaagaaatttgacac

SEQ ID NO: 19 - DNA, 43 bp, Artificial Sequence
  REGX1.1 Primer
ttccgtgtaccaagcaatttcatgtgacactaagaggggtgta

SEQ ID NO: 20 - DNA, 42 bp, Artificial Sequence
  REGX1.1 Primer
aagagctatgaattgcagacacctggacattccccattgaag

SEQ ID NO: 21 - DNA, 24 bp, Artificial Sequence
  REGX1.2 Primer
ttccgtgtaccaagcaatttcatg

SEQ ID NO: 22 - DNA, 19 bp, Artificial Sequence
  REGX1.2 Primer
tgacactaagaggggtgta

SEQ ID NO: 23 - DNA, 25 bp, Artificial Sequence
  REGX1.2 Primer
ctgaaaagagctatgaattgcagac

SEQ ID NO: 24 - DNA, 18 bp, Artificial Sequence
  REGX1.2 Primer
ttggacattccccattga

SEQ ID NO: 25 - DNA, 18 bp, Artificial Sequence
  REGX1.2 Primer
gtccgaacaactggactt

SEQ ID NO: 26 - DNA, 25 bp, Artificial Sequence
  REGX1.2 Primer
gtcttgattatggaatttaagggaa

SEQ ID NO: 27 - DNA, 20 bp, Artificial Sequence
  REGX1.2 Primer
tcatgttcacggcagcagta

SEQ ID NO: 28 - DNA, 23 bp, Artificial Sequence
  Artificial
attggcaaagaaatttgacacct

SEQ ID NO: 29 - DNA, 43 bp, Artificial Sequence; identical to SEQ ID NO: 19
  REGX1.2 Primer

SEQ ID NO: 30 - DNA, 43 bp, Artificial Sequence
  REGX1.2 Primer
ctgaaaagagctatgaattgcagacttggacattccccattga

SEQ ID NO: 31 - DNA, 24 bp, Artificial Sequence
  REGX2.1 Primer
agccgcattaatcttcagttcatc

SEQ ID NO: 32 - DNA, 18 bp, Artificial Sequence
  REGX2.1 Primer
taagcgtgttgactggac

SEQ ID NO: 33 - DNA, 23 bp, Artificial Sequence
  REGX2.1 Primer
agaaaggttcaacacatggttgt

SEQ ID NO: 34 - DNA, 20 bp, Artificial Sequence
  REGX2.1 Primer
tagggttaccaatgtcgtga

SEQ ID NO: 35 - DNA, 18 bp, Artificial Sequence
  REGX2.1 Primer
ctgtccacgagtgctttg

SEQ ID NO: 36 - DNA, 22 bp, Artificial Sequence
  REGX2.1 Primer
tgaggtacacacttaatagctt

SEQ ID NO: 37 - DNA, 21 bp, Artificial Sequence
  REGX2.1 Primer
accaattataggatattcaat

SEQ ID NO: 38 - DNA, 21 bp, Artificial Sequence
  REGX2.1 Primer
agcagacaaattcccagttct

SEQ ID NO: 39 - DNA, 42 bp, Artificial Sequence
  REGX2.1 Primer
agccgcattaatcttcagttcatctaagcgtgttgactggac

SEQ ID NO: 40 - DNA, 43 bp, Artificial Sequence
  REGX2.1 Primer
agaaaggttcaacacatggttgttagggttaccaatgtcgtga

SEQ ID NO: 41 - DNA, 24 bp, Artificial Sequence
  REGX2.2 Primer
gccgcattaatcttcagttcatca

SEQ ID NO: 42 - DNA, 18 bp, Artificial Sequence
  REGX2.2 Primer
ttaagcgtgttgactgga

SEQ ID NO: 43 - DNA, 25 bp, Artificial Sequence
  REGX2.2 Primer
agaaaggttcaacacatggttgtta

SEQ ID NO: 44 - DNA, 19 bp, Artificial Sequence
  REGX2.2 Primer
ttagggttaccaatgtcgt

SEQ ID NO: 45 - DNA, 18 bp, Artificial Sequence
  REGX2.2 Primer
ctgtccacgagtgctttg

SEQ ID NO: 46 - DNA, 21 bp, Artificial Sequence
  REGX2.2 Primer
tgaggtacacacttaatagct

SEQ ID NO: 47 - DNA, 22 bp, Artificial Sequence
  REGX2.2 Primer
ccaattataggatattcaatag

SEQ ID NO: 48 - DNA, 25 bp, Artificial Sequence
  REGX2.2 Primer
tgcattattagcagacaaattccca

SEQ ID NO: 49 - DNA, 42 bp, Artificial Sequence
  REGX2.2 Primer
gccgcattaatcttcagttcatcattaagcgtgttgactgga

SEQ ID NO: 50 - DNA, 44 bp, Artificial Sequence
  REGX2.2 Primer
agaaaggttcaacacatggttgttattagggttaccaatgtcgt

SEQ ID NO: 51 - DNA, 24 bp, Artificial Sequence
  REGX2.3 Primer
gccgcattaatcttcagttcatca

SEQ ID NO: 52 - DNA, 18 bp, Artificial Sequence
  REGX2.3 Primer
ttaagcgtgttgactgga

SEQ ID NO: 53 - DNA, 24 bp, Artificial Sequence
  REGX2.3 Primer
agaaaggttcaacacatggttgtt

SEQ ID NO: 54 - DNA, 19 bp, Artificial Sequence
  REGX2.3 Primer
ttagggttaccaatgtcgt

SEQ ID NO: 55 - DNA, 18 bp, Artificial Sequence
  Artificial
ctgtccacgagtgctttg

SEQ ID NO: 56 - DNA, 21 bp, Artificial Sequence
  REGX2.3 Primer
tgaggtacacacttaatagct

SEQ ID NO: 57 - DNA, 22 bp, Artificial Sequence
  REGX2.3 Primer
ccaattataggatattcaatag

SEQ ID NO: 58 - DNA, 25 bp, Artificial Sequence
  REGX2.3 Primer
tgcattattagcagacaaattccca

SEQ ID NO: 59 - DNA, 42 bp, Artificial Sequence; identical to SEQ ID NO: 49
  REGX2.3 Primer

SEQ ID NO: 60 - DNA, 43 bp, Artificial Sequence
  REGX2.3 Primer
agaaaggttcaacacatggttgttttagggttaccaatgtcgt

SEQ ID NO: 61 - DNA, 24 bp, Artificial Sequence
  REGX2.4 Primer
gccgcattaatcttcagttcatca

SEQ ID NO: 62 - DNA, 19 bp, Artificial Sequence
  REGX2.4 Primer
ttaagcgtgttgactggac

SEQ ID NO: 63 - DNA, 24 bp, Artificial Sequence
  REGX2.4 Primer
agaaaggttcaacacatggttgtt

SEQ ID NO: 64 - DNA, 19 bp, Artificial Sequence
  REGX2.4 Primer
ttagggttaccaatgtcgt

SEQ ID NO: 65 - DNA, 18 bp, Artificial Sequence
  REGX2.4 Primer
ctgtccacgagtgctttg

SEQ ID NO: 66 - DNA, 21 bp, Artificial Sequence
  REGX2.4 Primer
tgaggtacacacttaatagct

SEQ ID NO: 67 - DNA, 21 bp, Artificial Sequence
  REGX2.4 Primer
ccaattataggatattcaata

SEQ ID NO: 68 - DNA, 25 bp, Artificial Sequence
  REGX2.4 Primer
tgcattattagcagacaaattccca

SEQ ID NO: 69 - DNA, 43 bp, Artificial Sequence
  REGX2.4 Primer
gccgcattaatcttcagttcatcattaagcgtgttgactggac

SEQ ID NO: 70 - DNA, 43 bp, Artificial Sequence; identical to SEQ ID NO: 60
  REGX2.4 Primer

SEQ ID NO: 71 - DNA, 22 bp, Artificial Sequence
  N3 Primer
ccactgcgttctccattctggt

SEQ ID NO: 72 - DNA, 19 bp, Artificial Sequence
  N3 Primer
aaatgcaccccgcattacg

SEQ ID NO: 73 - DNA, 21 bp, Artificial Sequence
  N3 Primer
cgcgatcaaaacaacgtcggc

SEQ ID NO: 74 - DNA, 20 bp, Artificial Sequence
  N3 Primer
ccttgccatgttgagtgaga

SEQ ID NO: 75 - DNA, 18 bp, Artificial Sequence
  N3 Primer
tggaccccaaaatcagcg

SEQ ID NO: 76 - DNA, 19 bp, Artificial Sequence
  N3 Primer
gccttgtcctcgagggaat

SEQ ID NO: 77 - DNA, 21 bp, Artificial Sequence
  N3 Primer
gttgaatctgagggtccacca

SEQ ID NO: 78 - DNA, 22 bp, Artificial Sequence
  N3 Primer
acccaataatactgcgtcttgg

SEQ ID NO: 79 - DNA, 41 bp, Artificial Sequence
  N3 Primer
ccactgcgttctccattctggtaaatgcaccccgcattacg

SEQ ID NO: 80 - DNA, 41 bp, Artificial Sequence
  N3 Primer
cgcgatcaaaacaacgtcggcccttgccatgttgagtgaga

SEQ ID NO: 81 - DNA, 21 bp, Artificial Sequence
  N6 Primer
cgacgttgttttgatcgcgcc

SEQ ID NO: 82 - DNA, 20 bp, Artificial Sequence
  N6 Primer
attacgtttggtggaccctc

SEQ ID NO: 83 - DNA, 20 bp, Artificial Sequence
  N6 Primer
gcgtcttggttcaccgctct

SEQ ID NO: 84 - DNA, 20 bp, Artificial Sequence
  N6 Primer
aattggaacgccttgtcctc

SEQ ID NO: 85 - DNA, 20 bp, Artificial Sequence
  N6 Primer
ccccaaaatcagcgaaatgc

SEQ ID NO: 86 - DNA, 20 bp, Artificial Sequence
  N6 Primer
agccaatttggtcatctgga

SEQ ID NO: 87 - DNA, 23 bp, Artificial Sequence
  N6 Primer
tccattctggttactgccagttg

SEQ ID NO: 88 - DNA, 21 bp, Artificial Sequence
  N6 Primer
caacatggcaaggaagacctt

SEQ ID NO: 89 - DNA, 41 bp, Artificial Sequence
  N6 Primer
cgacgttgttttgatcgcgccattacgtttggtggaccctc

SEQ ID NO: 90 - DNA, 40 bp, Artificial Sequence
  N6 Primer
gcgtcttggttcaccgctctaattggaacgccttgtcctc

SEQ ID NO: 91 - DNA, 21 bp, Artificial Sequence
  N10 Primer
cgccttgtcctcgagggaatt

SEQ ID NO: 92 - DNA, 18 bp, Artificial Sequence
  N10 Primer
cgtcttggttcaccgctc

SEQ ID NO: 93 - DNA, 22 bp, Artificial Sequence
  N10 Primer
agacgaattcgtggtggtgacg

SEQ ID NO: 94 - DNA, 19 bp, Artificial Sequence
  N10 Primer
tggcccagttcctaggtag

SEQ ID NO: 95 - DNA, 19 bp, Artificial Sequence
  N10 Primer
gccccaaggtttacccaat

SEQ ID NO: 96 - DNA, 20 bp, Artificial Sequence
  N10 Primer
agcaccatagggaagtccag

SEQ ID NO: 97 - DNA, 21 bp, Artificial Sequence
  N10 Primer
tcttccttgccatgttgagtg

SEQ ID NO: 98 - DNA, 24 bp, Artificial Sequence
  N10 Primer
atgaaagatctcagtccaagatgg

SEQ ID NO: 99 - DNA, 39 bp, Artificial Sequence
  N10 Primer
cgccttgtcctcgagggaattcgtcttggttcaccgctc

SEQ ID NO: 100 - DNA, 41 bp, Artificial Sequence
  N10 Primer
agacgaattcgtggtggtgacgtggcccagttcctaggtag

SEQ ID NO: 101 - DNA, 27 bp, Artificial Sequence
  N13e Primer
gtctttgttagcaccatagggaagtcc

SEQ ID NO: 102 - DNA, 23 bp, Artificial Sequence
  N13e Primer
tgaaagatctcagtccaagatgg

SEQ ID NO: 103 - DNA, 27 bp, Artificial Sequence
  N13e Primer
ggagccttgaatacaccaaaagatcac

SEQ ID NO: 104 - DNA, 23 bp, Artificial Sequence
  N13e Primer
ttgaggaagttgtagcacgattg

SEQ ID NO: 105 - DNA, 23 bp, Artificial Sequence
  N13e Primer
aattggctactaccgaagagcta

SEQ ID NO: 106 - DNA, 23 bp, Artificial Sequence
  N13e Primer
gtagaagccttttggcaatgttg

SEQ ID NO: 107 - DNA, 27 bp, Artificial Sequence
  N13e Primer
tggcccagttcctaggtagtagaaata

SEQ ID NO: 108 - DNA, 22 bp, Artificial Sequence
  N13e Primer
cgcaatcctgctaacaatgctg

SEQ ID NO: 109 - DNA, 50 bp, Artificial Sequence
  N13e Primer
gtctttgttagcaccatagggaagtcctgaaagatctcagtccaagatgg

SEQ ID NO: 110 - DNA, 50 bp, Artificial Sequence
  N13e Primer
ggagccttgaatacaccaaaagatcacttgaggaagttgtagcacgattg

SEQ ID NO: 111 - DNA, 25 bp, Artificial Sequence
  RdRp.1 Primer
cagttgaaactacaaatggaacacc

SEQ ID NO: 112 - DNA, 19 bp, Artificial Sequence
  RdRp.1 Primer
tacagtgttcccacctaca

SEQ ID NO: 113 - DNA, 25 bp, Artificial Sequence
  RdRp.1 Primer
agctaggtgttgtacataatcagga

SEQ ID NO: 114 - DNA, 19 bp, Artificial Sequence
  RdRp.1 Primer
ggtcagcagcatacacaag

SEQ ID NO: 115 - DNA, 19 bp, Artificial Sequence
  RdRp.1 Primer
cagatgcattctgcattgt

SEQ ID NO: 116 - DNA, 18 bp, Artificial Sequence
  RdRp.1 Primer
attaccagaagcagcgtg

SEQ ID NO: 117 - DNA, 24 bp, Artificial Sequence
  RdRp.1 Primer
ttttctcactagtggtccaaaact

SEQ ID NO: 118 - DNA, 25 bp, Artificial Sequence
  RdRp.1 Primer
tgtaaacttacatagctctagactt

SEQ ID NO: 119 - DNA, 44 bp, Artificial Sequence
  RdRp.1 Primer
cagttgaaactacaaatggaacacctacagtgttcccacctaca

SEQ ID NO: 120 - DNA, 44 bp, Artificial Sequence
  RdRp.1 Primer
agctaggtgttgtacataatcaggaggtcagcagcatacacaag

SEQ ID NO: 121 - DNA, 22 bp, Artificial Sequence
  RdRp.2 Primer
gccaaccaccatagaatttgct

SEQ ID NO: 122 - DNA, 18 bp, Artificial Sequence
  RdRp.2 Primer
aatagccgccactagagg

SEQ ID NO: 123 - DNA, 23 bp, Artificial Sequence
  RdRp.2 Primer
agtgatgtagaaaaccctcacct

SEQ ID NO: 124 - DNA, 19 bp, Artificial Sequence
  RdRp.2 Primer
aggcatggctctatcacat

SEQ ID NO: 125 - DNA, 23 bp, Artificial Sequence
  RdRp.2 Primer
actatgaccaatagacagtttca

SEQ ID NO: 126 - DNA, 21 bp, Artificial Sequence
  RdRp.2 Primer
ggccataattctaagcatgtt

SEQ ID NO: 127 - DNA, 20 bp, Artificial Sequence
  RdRp.2 Primer
gttccaattactacagtagc

SEQ ID NO: 128 - DNA, 20 bp, Artificial Sequence
  RdRp.2 Primer
atgggttgggattatcctaa

SEQ ID NO: 129 - DNA, 40 bp, Artificial Sequence
  RdRp.2 Primer
gccaaccaccatagaatttgctaatagccgccactagagg

SEQ ID NO: 130 - DNA, 42 bp, Artificial Sequence
  RdRp.2 Primer
agtgatgtagaaaaccctcacctaggcatggctctatcacat

SEQ ID NO: 131 - DNA, 24 bp, Artificial Sequence
  RdRp.3 Primer
atcaccctgtttaactagcattgt

SEQ ID NO: 132 - DNA, 20 bp, Artificial Sequence
  RdRp.3 Primer
tgaccttactaaaggacctc

SEQ ID NO: 133 - DNA, 23 bp, Artificial Sequence
  RdRp.3 Primer
tatgtgtaccttccttacccaga

SEQ ID NO: 134 - DNA, 24 bp, Artificial Sequence
  RdRp.3 Primer
ccatctgtttttacgatatcatct

SEQ ID NO: 135 - DNA, 20 bp, Artificial Sequence
  RdRp.3 Primer
gcaaaatgttggactgagac

SEQ ID NO: 136 - DNA, 22 bp, Artificial Sequence
  RdRp.3 Primer
gaaccgttcaatcataagtgta

SEQ ID NO: 137 - DNA, 20 bp, Artificial Sequence
  RdRp.3 Primer
atgttgagagcaaaattcat

SEQ ID NO: 138 - DNA, 21 bp, Artificial Sequence
  RdRp.3 Primer
tccatcaagaatcctaggggc

SEQ ID NO: 139 - DNA, 44 bp, Artificial Sequence
  RdRp.3 Primer
atcaccctgtttaactagcattgttgaccttactaaaggacctc

SEQ ID NO: 140 - DNA, 47 bp, Artificial Sequence
  RdRp.3 Primer
tatgtgtaccttccttacccagaccatctgtttttacgatatcatct

SEQ ID NO: 141 - DNA, 25 bp, Artificial Sequence
  RdRp.4 Primer
atgcgtaaaactcattcacaaagtc

SEQ ID NO: 142 - DNA, 22 bp, Artificial Sequence
  RdRp.4 Primer
caacacagactttatgagtgtc

SEQ ID NO: 143 - DNA, 22 bp, Artificial Sequence
  RdRp.4 Primer
tgatactctctgacgatgctgt

SEQ ID NO: 144 - DNA, 19 bp, Artificial Sequence
  RdRp.4 Primer
agccactagaccttgagat

SEQ ID NO: 145 - DNA, 20 bp, Artificial Sequence
  RdRp.4 Primer
cgataagtatgtccgcaatt

SEQ ID NO: 146 - DNA, 23 bp, Artificial Sequence
  RdRp.4 Primer
actgacttaaagttctttatgct

SEQ ID NO: 147 - DNA, 25 bp, Artificial Sequence
  RdRp.4 Primer
tgtgtcaacatctctatttctatag

SEQ ID NO: 148 - DNA, 24 bp, Artificial Sequence
  RdRp.4 Primer
tgtgtgtttcaatagcacttatgc

SEQ ID NO: 149 - DNA, 47 bp, Artificial Sequence
  RdRp.4 Primer
atgcgtaaaactcattcacaaagtccaacacagactttatgagtgtc

SEQ ID NO: 150 - DNA, 41 bp, Artificial Sequence
  RdRp.4 Primer
tgatactctctgacgatgctgtagccactagaccttgagat

SEQ ID NO: 151 - DNA, 25 bp, Artificial Sequence
  Orf1ab.1 Primer
tcccccactagctagataatctttg

SEQ ID NO: 152 - DNA, 25 bp, Artificial Sequence
  Orf1ab.1 Primer
ccaattcaactgtattatctttctg

SEQ ID NO: 153 - DNA, 25 bp, Artificial Sequence
  Orf1ab.1 Primer
gtgttaagatgttgtgtacacacac

SEQ ID NO: 154 - DNA, 18 bp, Artificial Sequence
  Orf1ab.1 Primer
atccatattggcttccgg

SEQ ID NO: 155 - DNA, 19 bp, Artificial Sequence
  Orf1ab.1 Primer
agctggtaatgcaacagaa

SEQ ID NO: 156 - DNA, 19 bp, Artificial Sequence
  Orf1ab.1 Primer
caccaccaaaggattcttg

SEQ ID NO: 157 - DNA, 21 bp, Artificial Sequence
  Orf1ab.1 Primer
gctttagcagcatctacagca

SEQ ID NO: 158 - DNA, 24 bp, Artificial Sequence
  Orf1ab.1 Primer
tggtactggtcaggcaataacagt

SEQ ID NO: 159 - DNA, 50 bp, Artificial Sequence
  Orf1ab.1 Primer
tcccccactagctagataatctttgccaattcaactgtattatctttctg

SEQ ID NO: 160 - DNA, 43 bp, Artificial Sequence
  Orf1ab.1 Primer
gtgttaagatgttgtgtacacacacatccatattggcttccgg

SEQ ID NO: 161 - DNA, 20 bp, Artificial Sequence
  Orf1ab.2 Primer
tgactgaagcatgggttcgc

SEQ ID NO: 162 - DNA, 19 bp, Artificial Sequence
  Orf1ab.2 Primer
gtctgcggtatgtggaaag

SEQ ID NO: 163 - DNA, 25 bp, Artificial Sequence
  Orf1ab.2 Primer
gctgatgcacaatcgtttttaaacg

SEQ ID NO: 164 - DNA, 19 bp, Artificial Sequence
  Orf1ab.2 Primer
catcagtactagtgcctgt

SEQ ID NO: 165 - DNA, 22 bp, Artificial Sequence
  Orf1ab.2 Primer
acttaaaaacacagtctgtacc

SEQ ID NO: 166 - DNA, 19 bp, Artificial Sequence
  Orf1ab.2 Primer
tcaaaagccctgtatacga

SEQ ID NO: 167 - DNA, 24 bp, Artificial Sequence
  Orf1ab.2 Primer
gagttgatcacaactacagccata

SEQ ID NO: 168 - DNA, 19 bp, Artificial Sequence
  Orf1ab.2 Primer
ttgcggtgtaagtgcagcc

SEQ ID NO: 169 - DNA, 39 bp, Artificial Sequence
  Orf1ab.2 Primer
tgactgaagcatgggttcgcgtctgcggtatgtggaaag

SEQ ID NO: 170 - DNA, 44 bp, Artificial Sequence
  Orf1ab.2 Primer
gctgatgcacaatcgtttttaaacgcatcagtactagtgcctgt

SEQ ID NO: 171 - DNA, 25 bp, Artificial Sequence
  Orf1ab.3 Primer
gatcacaactacagccataaccttt

SEQ ID NO: 172 - DNA, 23 bp, Artificial Sequence
  Orf1ab.3 Primer
gggttttacacttaaaaacacag

SEQ ID NO: 173 - DNA, 24 bp, Artificial Sequence
  Orf1ab.3 Primer
tgatgcacaatcgtttttaaacgg

SEQ ID NO: 174 - DNA, 19 bp, Artificial Sequence
  Orf1ab.3 Primer
catcagtactagtgcctgt

SEQ ID NO: 175 - DNA, 18 bp, Artificial Sequence
  Orf1ab.3 Primer
ttgtgctaatgaccctgt

SEQ ID NO: 176 - DNA, 19 bp, Artificial Sequence
  Orf1ab.3 Primer
tcaaaagccctgtatacga

SEQ ID NO: 177 - DNA, 22 bp, Artificial Sequence
  Orf1ab.3 Primer
ccacataccgcagacggtacag

SEQ ID NO: 178 - DNA, 18 bp, Artificial Sequence
  Orf1ab.3 Primer
ggtgtaagtgcagcccgt

SEQ ID NO: 179 - DNA, 48 bp, Artificial Sequence
  Orf1ab.3 Primer
gatcacaactacagccataacctttgggttttacacttaaaaacacag

SEQ ID NO: 180 - DNA, 43 bp, Artificial Sequence
  Orf1ab.3 Primer
tgatgcacaatcgtttttaaacggcatcagtactagtgcctgt

SEQ ID NO: 181 - DNA, 22 bp, Artificial Sequence
  Orf1ab.4 Primer
acaaggtggttccagttctgta

SEQ ID NO: 182 - DNA, 20 bp, Artificial Sequence
  Orf1ab.4 Primer
gggctagattccctaagagt

SEQ ID NO: 183 - DNA, 24 bp, Artificial Sequence
  Orf1ab.4 Primer
tgttacagacacacctaaaggtcc

SEQ ID NO: 184 - DNA, 23 bp, Artificial Sequence
  Orf1ab.4 Primer
accatacctctatttaggttgtt

SEQ ID NO: 185 - DNA, 22 bp, Artificial Sequence
  Orf1ab.4 Primer
ctgttatccgatttacaggatt

SEQ ID NO: 186 - DNA, 19 bp, Artificial Sequence
  Orf1ab.4 Primer
ggcagctaaactaccaagt

SEQ ID NO: 187 - DNA, 20 bp, Artificial Sequence
  Orf1ab.4 Primer
tagatagtaccagttccatc

SEQ ID NO: 188 - DNA, 25 bp, Artificial Sequence
  Orf1ab.4 Primer
tgaagtatttatactttattaaagg

SEQ ID NO: 189 - DNA, 42 bp, Artificial Sequence
  Orf1ab.4 Primer
acaaggtggttccagttctgtagggctagattccctaagagt

SEQ ID NO: 190 - DNA, 47 bp, Artificial Sequence
  Orf1ab.4 Primer
tgttacagacacacctaaaggtccaccatacctctatttaggttgtt

SEQ ID NO: 191 - DNA, 24 bp, Artificial Sequence
  E.1 Primer
cgtcggttcatcataaattggttc

SEQ ID NO: 192 - DNA, 19 bp, Artificial Sequence
  E.1 Primer
cacaatcgacggttcatcc

SEQ ID NO: 193 - DNA, 23 bp, Artificial Sequence
  E.1 Primer
actactagcgtgcctttgtaagc

SEQ ID NO: 194 - DNA, 19 bp, Artificial Sequence
  E.1 Primer
gtctcttccgaaacgaatg

SEQ ID NO: 195 - DNA, 20 bp, Artificial Sequence
  E.1 Primer
cctgaagaacatgtccaaat

SEQ ID NO: 196 - DNA, 23 bp, Artificial Sequence
  E.1 Primer
cgctattaactattaacgtacct

SEQ ID NO: 197 - DNA, 21 bp, Artificial Sequence
  E.1 Primer
cattactggattaacaactcc

SEQ ID NO: 198 - DNA, 25 bp, Artificial Sequence
  E.1 Primer
acaagctgatgagtacgaacttatg

SEQ ID NO: 199 - DNA, 43 bp, Artificial Sequence
  E.1 Primer
cgtcggttcatcataaattggttccacaatcgacggttcatcc

SEQ ID NO: 200 - DNA, 42 bp, Artificial Sequence
  E.1 Primer
actactagcgtgcctttgtaagcgtctcttccgaaacgaatg

SEQ ID NO: 201 - DNA, 25 bp, Artificial Sequence
  E.2 Primer
cgaaagcaagaaaaagaagtacgct

SEQ ID NO: 202 - DNA, 24 bp, Artificial Sequence
  E.2 Primer
agtacgaacttatgtactcattcg

SEQ ID NO: 203 - DNA, 25 bp, Artificial Sequence
  E.2 Primer
tggtattcttgctagttacactagc

SEQ ID NO: 204 - DNA, 22 bp, Artificial Sequence
  E.2 Primer
agactcacgttaacaatattgc

SEQ ID NO: 205 - DNA, 19 bp, Artificial Sequence
  E.2 Primer
ttgtaagcacaagctgatg

SEQ ID NO: 206 - DNA, 23 bp, Artificial Sequence
  E.2 Primer
agagtaaacgtaaaaagaaggtt

SEQ ID NO: 207 - DNA, 21 bp, Artificial Sequence
  E.2 Primer
acgtacctgtctcttccgaaa

SEQ ID NO: 208 - DNA, 24 bp, Artificial Sequence
  E.2 Primer
catccttactgcgcttcgattgtg

SEQ ID NO: 209 - DNA, 49 bp, Artificial Sequence
  E.2 Primer
cgaaagcaagaaaaagaagtacgctagtacgaacttatgtactcattcg

SEQ ID NO: 210 - DNA, 47 bp, Artificial Sequence
  E.2 Primer
tggtattcttgctagttacactagcagactcacgttaacaatattgc

SEQ ID NO: 211 - DNA, 25 bp, Artificial Sequence
  E.3 Primer
ctagcaagaataccacgaaagcaag

SEQ ID NO: 212 - DNA, 19 bp, Artificial Sequence
  E.3 Primer
ttcggaagagacaggtacg

SEQ ID NO: 213 - DNA, 21 bp, Artificial Sequence
  E.3 Primer
cactagccatccttactgcgc

SEQ ID NO: 214 - DNA, 21 bp, Artificial Sequence
  E.3 Primer
aaggttttacaagactcacgt

SEQ ID NO: 215 - DNA, 23 bp, Artificial Sequence
  E.3 Primer
gtacgaacttatgtactcattcg

SEQ ID NO: 216 - DNA, 22 bp, Artificial Sequence
  E.3 Primer
tttttaacacgagagtaaacgt

SEQ ID NO: 217 - DNA, 22 bp, Artificial Sequence
  E.3 Primer
agaagtacgctattaactatta

SEQ ID NO: 218 - DNA, 22 bp, Artificial Sequence
  E.3 Primer
ttcgattgtgtgcgtactgctg

SEQ ID NO: 219 - DNA, 44 bp, Artificial Sequence
  E.3 Primer
ctagcaagaataccacgaaagcaagttcggaagagacaggtacg

SEQ ID NO: 220 - DNA, 42 bp, Artificial Sequence
  E.3 Primer
cactagccatccttactgcgcaaggttttacaagactcacgt

SEQ ID NO: 221 - DNA, 25 bp, Artificial Sequence
  E.4 Pimer
acgagagtaaacgtaaaaagaaggt

SEQ ID NO: 222 - DNA, 18 bp, Artificial Sequence
  E.4 Pimer
gcttcgattgtgtgcgta

SEQ ID NO: 223 - DNA, 24 bp, Artificial Sequence
  E.4 Pimer
ctagagttcctgatcttctggtct

SEQ ID NO: 224 - DNA, 23 bp, Artificial Sequence
  E.4 Pimer
tggctaaaattaaagttccaaac

SEQ ID NO: 225 - DNA, 19 bp, Artificial Sequence
  E.4 Pimer
cactagccatccttactgc

SEQ ID NO: 226 - DNA, 18 bp, Artificial Sequence
  E.4 Pimer
gtaccgttggaatctgcc

SEQ ID NO: 227 - DNA, 25 bp, Artificial Sequence
  E.4 Pimer
agactcacgttaacaatattgcagc

SEQ ID NO: 228 - DNA, 25 bp, Artificial Sequence
  E.4 Pimer
acgaactaaatattatattagtttt

SEQ ID NO: 229 - DNA, 43 bp, Artificial Sequence
  E.4 Pimer
acgagagtaaacgtaaaaagaaggtgcttcgattgtgtgcgta

SEQ ID NO: 230 - DNA, 47 bp, Artificial Sequence
  E.4 Pimer
ctagagttcctgatcttctggtcttggctaaaattaaagttccaaac

SEQ ID NO: 231 - DNA, 25 bp, Artificial Sequence
  E.2 Primer
ctgccatggctaaaattaaagttcc

SEQ ID NO: 232 - DNA, 20 bp, Artificial Sequence
  E.2 Primer
agttcctgatcttctggtct

SEQ ID NO: 233 - DNA, 23 bp, Artificial Sequence
  E.2 Primer
tccaacggtactattaccgttga

SEQ ID NO: 234 - DNA, 25 bp, Artificial Sequence
  E.2 Primer
aaggaataggaaacctattactagg

SEQ ID NO: 235 - DNA, 22 bp, Artificial Sequence
  E.2 Primer
actctcgtgttaaaaatctgaa

SEQ ID NO: 236 - DNA, 24 bp, Artificial Sequence
  E.2 Primer
gcaaattgtagaagacaaatccat

SEQ ID NO: 237 - DNA, 25 bp, Artificial Sequence
  E.2 Primer
aaaactaatataatatttagttcgt

SEQ ID NO: 238 - DNA, 23 bp, Artificial Sequence
  E.2 Primer
aaaaagctccttgaacaatggaa

SEQ ID NO: 239 - DNA, 45 bp, Artificial Sequence
  E.2 Primer
ctgccatggctaaaattaaagttccagttcctgatcttctggtct

SEQ ID NO: 240 - DNA, 48 bp, Artificial Sequence
  E.2 Primer
tccaacggtactattaccgttgaaaggaataggaaacctattactagg

SEQ ID NO: 241 - DNA, 21 bp, Artificial Sequence
  RNaseP.1 Primer
gttgcggatccgagtcagtgg

SEQ ID NO: 242 - DNA, 19 bp, Artificial Sequence
  RNaseP.1 Primer
ccgtggagcttgttgatga

SEQ ID NO: 243 - DNA, 22 bp, Artificial Sequence
  RNaseP.1 Primer
aactcagccatccacatccgag

SEQ ID NO: 244 - DNA, 19 bp, Artificial Sequence
  RNaseP.1 Primer
tcacggaggggataagtgg

SEQ ID NO: 245 - DNA, 18 bp, Artificial Sequence
  RNaseP.1 Primer
ggtggctgccaatacctc

SEQ ID NO: 246 - DNA, 18 bp, Artificial Sequence
  RNaseP.1 Primer
actcagcatgcgaagagc

SEQ ID NO: 247 - DNA, 21 bp, Artificial Sequence
  RNaseP.1 Primer
gtgtgtcggtctctggctcca

SEQ ID NO: 248 - DNA, 21 bp, Artificial Sequence
  RNaseP.1 Primer
tcttcagggtcacacccaagt

SEQ ID NO: 249 - DNA, 40 bp, Artificial Sequence
  RNaseP.1 Primer
gttgcggatccgagtcagtggccgtggagcttgttgatga

SEQ ID NO: 250 - DNA, 41 bp, Artificial Sequence
  RNaseP.1 Primer
aactcagccatccacatccgagtcacggaggggataagtgg

SEQ ID NO: 251 - DNA, 22 bp, Artificial Sequence
  RNaseP.2 Primer
cggatgtggatggctgagttgt

SEQ ID NO: 252 - DNA, 18 bp, Artificial Sequence
  RNaseP.2 Primer
gagccagagaccgacaca

SEQ ID NO: 253 - DNA, 22 bp, Artificial Sequence
  RNaseP.2 Primer
actcctccacttatcccctccg

SEQ ID NO: 254 - DNA, 18 bp, Artificial Sequence
  RNaseP.2 Primer
tggtccgaggtccagtac

SEQ ID NO: 255 - DNA, 20 bp, Artificial Sequence
  RNaseP.2 Primer
cgtggagcttgttgatgagc

SEQ ID NO: 256 - DNA, 18 bp, Artificial Sequence
  RNaseP.2 Primer
tgggcttccagggaacag

SEQ ID NO: 257 - DNA, 20 bp, Artificial Sequence
  RNaseP.2 Primer
atccgagtcagtggctcccg

SEQ ID NO: 258 - DNA, 20 bp, Artificial Sequence
  RNaseP.2 Primer
atatggctcttcgcatgctg

SEQ ID NO: 259 - DNA, 40 bp, Artificial Sequence
  RNaseP.2 Primer
cggatgtggatggctgagttgtgagccagagaccgacaca

SEQ ID NO: 260 - DNA, 40 bp, Artificial Sequence
  RNaseP.2 Primer
actcctccacttatcccctccgtggtccgaggtccagtac

SEQ ID NO: 261 - DNA, 21 bp, Artificial Sequence
  RNaseP.3 Primer
acatggctctggtccgaggtc

SEQ ID NO: 262 - DNA, 20 bp, Artificial Sequence
  RNaseP.3 Primer
ctccacttatcccctccgtg

SEQ ID NO: 263 - DNA, 22 bp, Artificial Sequence
  RNaseP.3 Primer
ctgttccctggaagcccaaagg

SEQ ID NO: 264 - DNA, 19 bp, Artificial Sequence
  RNaseP.3 Primer
taactgggcccaccaagag

SEQ ID NO: 265 - DNA, 18 bp, Artificial Sequence
  RNaseP.3 Primer
tcagggtcacacccaagt

SEQ ID NO: 266 - DNA, 20 bp, Artificial Sequence
  RNaseP.3 Primer
cgcatacacacactcaggaa

SEQ ID NO: 267 - DNA, 23 bp, Artificial Sequence
  RNaseP.3 Primer
actcagcatgcgaagagccatat

SEQ ID NO: 268 - DNA, 22 bp, Artificial Sequence
  RNaseP.3 Primer
ctgcattgagggtgggggtaat

SEQ ID NO: 269 - DNA, 41 bp, Artificial Sequence
  RNaseP.3 Primer
acatggctctggtccgaggtcctccacttatcccctccgtg

SEQ ID NO: 270 - DNA, 41 bp, Artificial Sequence
  RNaseP.3 Primer
ctgttccctggaagcccaaaggtaactgggcccaccaagag

SEQ ID NO: 271 - DNA, 22 bp, Artificial Sequence
  RNaseP.4 Primer
cactggatccagttcagcctcc

SEQ ID NO: 272 - DNA, 18 bp, Artificial Sequence
  RNaseP.4 Primer
gcacacagcatggcagaa

SEQ ID NO: 273 - DNA, 22 bp, Artificial Sequence
  RNaseP.4 Primer
ttaggaaaaggcttcccagccg

SEQ ID NO: 274 - DNA, 20 bp, Artificial Sequence
  RNaseP.4 Primer
tgggccttaaagtccgtctt

SEQ ID NO: 275 - DNA, 18 bp, Artificial Sequence
  RNaseP.4 Primer
gccctgtggaacgaagag

SEQ ID NO: 276 - DNA, 18 bp, Artificial Sequence
  RNaseP.4 Primer
tccgtccagcagcttctg

SEQ ID NO: 277 - DNA, 18 bp, Artificial Sequence
  RNaseP.4 Primer
caccgcggggctctcggt

SEQ ID NO: 278 - DNA, 19 bp, Artificial Sequence
  RNaseP.4 Primer
ctgccccggagacccaatg

SEQ ID NO: 279 - DNA, 40 bp, Artificial Sequence
  RNaseP.4 Primer
cactggatccagttcagcctccgcacacagcatggcagaa

SEQ ID NO: 280 - DNA, 42 bp, Artificial Sequence
  RNaseP.4 Primer
ttaggaaaaggcttcccagccgtgggccttaaagtccgtctt

SEQ ID NO: 281 - DNA, 20 bp, Artificial Sequence
  RNaseP.5 Primer
cacctgcaaggacccgaagc

SEQ ID NO: 282 - DNA, 18 bp, Artificial Sequence
  RNaseP.5 Primer
aaccgcgccatcaacatc

SEQ ID NO: 283 - DNA, 21 bp, Artificial Sequence
  RNaseP.5 Primer
gccaatacctccaccgtggag

SEQ ID NO: 284 - DNA, 18 bp, Artificial Sequence
  RNaseP.5 Primer
gttgcggatccgagtcag

SEQ ID NO: 285 - DNA, 18 bp, Artificial Sequence
  RNaseP.5 Primer
tacattcacggcttgggc

SEQ ID NO: 286 - DNA, 19 bp, Artificial Sequence
  RNaseP.5 Primer
gggtgtgaccctgaagact

SEQ ID NO: 287 - DNA, 18 bp, Artificial Sequence
  RNaseP.5 Primer
cgcctgcagctgcagcgc

SEQ ID NO: 288 - DNA, 22 bp, Artificial Sequence
  RNaseP.5 Primer
gttgatgagctggagccagaga

SEQ ID NO: 289 - DNA, 38 bp, Artificial Sequence
  RNaseP.5 Primer
cacctgcaaggacccgaagcaaccgcgccatcaacatc

SEQ ID NO: 290 - DNA, 39 bp, Artificial Sequence
  RNaseP.5 Primer
gccaatacctccaccgtggaggttgcggatccgagtcag

(19) United States
(12) Patent Application Publication
(10) Pub. No.: US 2022/0290261 A1
(43) Pub. Date: Sep. 15, 2022

(54) PRIMER DESIGN AND USE FOR LOOP-MEDIATED ISOTHERMAL AMPLIFICATION (LAMP) PATHOGEN DETECTION

(71) Applicant: Purdue Research Foundation, West Lafayette, IN (US)

(72) Inventors: Josiah Davidson, West Lafayette, IN (US); Jiangshan Wang, West Lafayette, IN (US); Murali Kannan Maruthamuthu, Lafayette, IN (US); Andres Dextre, West Lafayette, IN (US); Mohit Verma, West Lafayette, IN (US)

(21) Appl. No.: 17/670,091

(22) Filed: Feb. 11, 2022

Related U.S. Application Data
(60) Provisional application No. 63/148,527, filed on Feb. 11, 2021.

(51) Int. Cl. C12Q 1/70; C12Q 1/6811; G01N 33/52

(52) U.S. Cl. CPC C12Q 1/701; C12Q 1/6811; G01N 33/523; C12Q 1/6853

(57) ABSTRACT

The present disclosure is drawn to an isolated complementary DNA (cDNA) of a nucleic acid molecule that can comprise a nucleotide sequence that is at least 85% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or a combination thereof. In one embodiment, a primer set for reverse transcription loop-mediated isothermal amplification (RT-LAMP) analysis can comprise a forward inner primer (FIP) sequence, a backward inner primer (BIP) sequence, a forward outer primer (F3) sequence, a backward outer primer (B3) sequence, a forward loop primer (LF) sequence, and a backward loop primer (LB) sequence. In another embodiment, a method of detecting a target pathogen can comprise providing a primer set.

### RELATED APPLICATIONS

This application claims the benefit of U.S. Provisional Patent Application Ser. No. 63/148,527 filed Feb. 11, 2021, the entire contents of which are incorporated herein by reference.

### BACKGROUND

Polymerase chain reaction (PCR) is a molecular biology technique that allows amplification of nucleotides for various analytical purposes. Quantitative PCR (qPCR) is an adaptation of PCR which allows monitoring of the amplification of a targeted nucleotide during the PCR. Diagnostic qPCR has been applied to detect nucleotides that are diagnostic of infectious diseases, cancer, and genetic abnormalities. Reverse transcriptase qPCR (RT-qPCR) is an adaptation of qPCR which allows detection of a target RNA nucleotide. Because of this ability, RT-qPCR is well-suited for detecting virus pathogens. However, RT-qPCR requires sizeable conventional equipment which may not be available in certain point of care settings, and additionally requires significant sample preparation and time to perform and obtain results.

By contrast, Loop-Mediated Isothermal Amplification (LAMP) is a more simplistic approach to diagnostic identification of target nucleotides. In particular, LAMP is a one-operation nucleic acid amplification method to multiply specific target nucleotide sequences. In addition to use of an isothermal heating process, LAMP can use a visual output test indicator, such as a simple color change rather than a more complicated fluorescent indicator required by PCR. Reverse-transcriptase LAMP (RT-LAMP) can be used like RT-qPCR in order to identify the presence or absence target nucleotides from RNA, and as such, can be used in a diagnostic capacity to identify the presence or absence of viral pathogens in a test subject. Because LAMP is a more simplistic, it can be performed with less equipment and sample preparation and therefore is more accessible for use in point of care settings, such as clinics, emergency rooms, and even on a mobile basis.

### SUMMARY

The present disclosure is drawn to technology (e.g., cDNA, primer sets, and methods) for reverse transcription loop-mediated isothermal amplification (RT-LAMP) analysis and detecting a Sarbecovirus target pathogen in a subject.

In some disclosure embodiments, an isolated complementary DNA (cDNA) of a nucleic acid molecule can include a nucleotide sequence that is at least 85% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or a combination thereof. In one aspect, the nucleotide sequence can be at least 85% identical to SEQ ID NO: 9 (e.g., SEQ ID NO: 1 joined to SEQ ID NO: 2). In yet another aspect, the nucleotide sequence can comprise SEQ ID NO: 1 joined to SEQ ID NO: 2 by a linking sequence selected from Table 11. In a further aspect, the nucleotide sequence can be at least 85% identical to SEQ ID NO: 10 (e.g., SEQ ID NO:3 joined to SEQ ID NO: 4). In yet another aspect, the nucleotide sequence can comprise SEQ ID NO: 3 joined to SEQ ID NO: 4 by a linking sequence selected from Table 11.

In one aspect, the guanine and cytosine (GC) content of the nucleotide sequence can be 50% or less. In another aspect, the guanine and cytosine (GC) content of the nucleotide sequence can be 40% or less. In another aspect, an end stability of the nucleotide sequence can be less than −2.5 kcal/mol. In another aspect, the nucleotide sequence can have a melting temperature of from about 40° C. to about 62° C. In yet another aspect, the nucleotide sequence can have a minimum primer dimerization energy of less than −1.0 kcal/mol. In yet another aspect, the nucleotide sequence can be less than 50% identical to nucleotide sequences associated with non-target agents (commensal microorganisms, other pathogens, and human genome).

In another aspect, the nucleotide sequence can be at least 90% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or a combination thereof. In another aspect, the nucleotide sequence can be at least 95% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or a combination thereof. In another aspect, the nucleotide sequence can be 100% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 or a combination thereof.

In some disclosure embodiments, a primer set for reverse transcription loop-mediated isothermal amplification (RT-LAMP) analysis can include: a forward inner primer (FIP) sequence that is at least 85% identical to a combination of SEQ ID NO: 1 coupled to SEQ ID NO: 2 (e.g. SEQ ID NO: 9); a backward inner primer (BIP) sequence that is at least 85% identical to a combination of seq ID NO: 3 coupled to SEQ ID NO: 4 (e.g. SEQ ID NO: 10); a forward outer primer (F3) sequence that is at least 85% identical to SEQ ID NO: 5; a backward outer primer (B3) sequence that is at least 85% identical to SEQ ID NO: 6; a forward loop primer (LF) sequence that is at least 85% identical to SEQ ID NO: 7; and a backward loop primer (LB) sequence that is at least 85% identical to SEQ ID NO: 8.

In one aspect, the FIP sequence can include a linking sequence joining SEQ ID NO: 1 and SEQ ID NO: 2. In one aspect, the linking sequence can be selected from Table 11. In another aspect, the BIP sequence can include a linking sequence joining SEQ ID NO: 3 and SEQ ID NO: 4. In one aspect, the linking sequence can be selected from Table 11.

In another aspect, the guanine and cytosine (GC) content of the FIP, the BIP, the F3, the B3, the LF, the LB, or a combination thereof can be 50% or less. In another aspect, the guanine and cytosine (GC) content of the FIP, the BIP, the F3, the B3, the LF, the LB, or a combination thereof can be 40% or less. In yet another aspect, an end stability of the FIP, the BIP, the F3, the B3, the LF, the LB, or a combination thereof can be less than −2.5 kcal/mol. In another aspect, the FIP, the BIP, the F3, the B3, the LF, the LB, or a combination thereof can have a melting temperature of from about 40° C. to about 62° C. In yet another aspect, the FIP, the BIP, the F3, the B3, the LF, the LB, or a combination thereof can have a minimum primer dimerization energy of less than −1.0 kcal/mol. In another aspect, the FIP, the BIP, the F3, the B3, the LF, the LB, or a combination thereof can have less than 50% identical to nucleotide sequences associated with non-target agents (commensal microorganisms, other pathogens, and human genome).

In another aspect, the FIP sequence can be at least 90% identical to a combination of SEQ ID NO: 1 and SEQ ID NO: 2. In another aspect, the BIP sequence can be at least 90% identical to a combination of seq ID NO: 3 and SEQ ID NO: 4. In another aspect, the F3 sequence can be at least 90% identical to SEQ ID NO: 5. In another aspect, the B3 sequence can be at least 90% identical to SEQ ID NO: 6. In another aspect, the LF sequence can be at least 90% identical to SEQ ID NO: 7. In another aspect, the LB sequence can be at least 90% identical to SEQ ID NO: 8.

In another aspect, the FIP sequence can be at least 95% identical to a combination of SEQ ID NO: 1 and SEQ ID NO: 2. In another aspect, the BIP sequence can be at least 95% identical to a combination of seq ID NO: 3 and SEQ ID NO: 4. In another aspect, the F3 sequence can be at least 95% identical to SEQ ID NO: 5. In another aspect, the B3 sequence can be at least 95% identical to SEQ ID NO: 6. In another aspect, the LF sequence can be at least 95% identical to SEQ ID NO: 7. In another aspect, the LB sequence can be at least 95% identical to SEQ ID NO: 8.

In yet another aspect, the FIP sequence can be at least 100% identical to a combination of SEQ ID NO: 1 and SEQ ID NO: 2, which is equivalent to SEQ ID NO: 9. In another aspect, the BIP sequence can be at least 100% identical to a combination of seq ID NO: 3 and SEQ ID NO: 4, which is equivalent to SEQ ID NO: 10. In another aspect, the F3 sequence can be at least 100% identical to SEQ ID NO: 5. In another aspect, the B3 sequence can be at least 100% identical to SEQ ID NO: 6. In another aspect, the LF sequence can be at least 100% identical to SEQ ID NO: 7. In another aspect, the LB sequence can be at least 100% identical to SEQ ID NO: 8.

In some disclosure embodiments, a method of detecting a target Sarbecovirus pathogen in a subject can include providing a primer set. In one aspect, the primer set can include: a forward inner primer (FIP) sequence that is at least 85% identical to a combination of SEQ ID NO: 1 and SEQ ID NO: 2; a backward inner primer (BIP) sequence that is at least 85% identical to a combination of seq ID NO: 3 and SEQ ID NO: 4; a forward outer primer (F3) sequence that is at least 85% identical to SEQ ID NO: 5; a backward outer primer (B3) sequence that is at least 85% identical to SEQ ID NO: 6; a forward loop primer (LF) sequence that is at least 85% identical to SEQ ID NO: 7; and a backward loop primer (LB) sequence that is at least 85% identical to SEQ ID NO: 8.

In one aspect, the target pathogen can be a human coronavirus selected from: Severe Acute Respiratory Syndrome (SARS)-CoV (SARS-CoV), Severe Acute Respiratory Syndrome (SARS)-CoV 2 (SARS-CoV-2), Middle East Respiratory Syndrome (MERS)-CoV (MERS-CoV), SARS-CoV hCoV-HKU1, hCoV-0C43, hCoV-NL63, and hCoV-229E. In one aspect, the subject can be a human subject. In yet another aspect, the target pathogen can be Severe Acute Respiratory Syndrome (SARS)-CoV 2 (SARS-CoV-2).

### BRIEF DESCRIPTION OF THE DRAWINGS

Features and advantages of the disclosure will be apparent from the detailed description which follows, taken in conjunction with the accompanying drawings, which together illustrate, by way of example, features of the disclosure; and, wherein:

FIG. 1 illustrates a schematic of target regions on a solid-reaction medium in accordance with an example embodiment;

FIG. 2 illustrates RT-qLAMP amplification curves for varying primer sets in saliva at a final concentration of 18%. Blue lines indicate positive control where 5 μL of heat-inactivated SARS-CoV-2 spiked into saliva was added to the reaction mix to result in a final concentration of 1.0×10⁵ viral genome copies per reaction. Black lines indicate non-template control (NTC) where 5 μL of saliva diluted 9:10 with water was added to the reaction mix in accordance with an example embodiment;

FIG. 3A illustrates RT-qLAMP amplification curves for varying primer sets in water. Blue lines indicate positive control where 5 μL of 0.2 ng/μL A) N gene synthetic RNA template, B) RNA-dependent RNA Polymerase (RdRP) synthetic RNA template, or C) orflab synthetic RNA template was added to the reaction. Black lines indicate non-template controls (NTC) where 5 μL of water was added in place of template synthetic RNA. Four replicates of each condition were run per primer set. Reactions had a final volume of 25 μL and used 2×NEB Fluorometric LAMP master mix per the manufacturer protocol. Reactions were run on a qTower3G with maximum ramp rate in accordance with an example embodiment;

FIG. 3B illustrates fluorometric screening of Region X primer sets in Saliva using Heat-inactivated SARS-CoV-2 in accordance with an example embodiment with RT-qLAMP fluorometric results of Region X primer sets in 18% saliva. Blue lines indicate positive controls where 5 μL of heat-inactivated SARS-CoV-2 were added to the reaction mix to result in a final concentration of 1.0×10⁵ viral genome copies per reaction. Black lines indicate non-template control (NTC) where 5 μL of human saliva was diluted to 90% with nuclease-free water and was added to the reaction mix. Reactions had a final volume of 25 μL and used NEB 2×Fluorometric master mix. Reactions were run on a qTower3G with a ramp rate of 0.1° C./s;

FIG. 4 illustrates Colorimetric RT-LAMP scan images for limit of detection (LoD) of orflab primer sets. Yellow wells indicate a successful LAMP reaction taking place whereas red/orange wells indicate absent or low-level amplifications respectively. 20 μL reaction mixtures were spiked with 5 μL of heat-inactivated virus dilutions in water at the labeled concentrations. Endpoint images were taken after incubating the plate at 65° C. for 60 minutes. Three replicates for each viral concentration were run per primer set in accordance with an example embodiment;

FIG. 5 illustrates Fluorometric RT-qLAMP results for primer sets targeting human RNaseP POP7 gene in A) 18% saliva spiked with 10⁵ genome equivalents/reaction of heat-inactivated SARS-CoV-2, and B) water with 0.2 ng of synthetic RNaseP POP7 RNA in accordance with an example embodiment;

FIG. 6 illustrates the limit of detection in fresh saliva for the orf7ab primer set in accordance with an example embodiment;

FIG. 7 illustrates the limit of detection for the orf7ab primer set in accordance with an example embodiment;

FIG. 8A illustrates a graph of intensity of fluorescence over time in accordance with an example embodiment;

FIG. 8B illustrates a graph of intensity of fluorescence over time in accordance with an example embodiment;

FIG. 9A illustrates a graph of intensity of fluorescence over time in accordance with an example embodiment;

FIG. 9B illustrates a graph of intensity of fluorescence over time in accordance with an example embodiment;

FIG. 9C illustrates a graph of intensity of fluorescence over time in accordance with an example embodiment;

FIG. 9D illustrates a graph of intensity of fluorescence over time in accordance with an example embodiment;

FIG. 9E illustrates a graph of intensity of fluorescence over time in accordance with an example embodiment;

FIG. 9F illustrates a graph of intensity of fluorescence over time in accordance with an example embodiment;

FIG. 9G illustrates a graph of intensity of fluorescence over time in accordance with an example embodiment;

FIG. 10A illustrates a graph of intensity of fluorescence over time in accordance with an example embodiment;

FIG. 10B illustrates a graph of intensity of fluorescence over time in accordance with an example embodiment;

FIG. 10C illustrates a graph of intensity of fluorescence over time in accordance with an example embodiment;

FIG. 10D illustrates a graph of intensity of fluorescence over time in accordance with an example embodiment;

FIG. 11A illustrates a graph of intensity of fluorescence over time in accordance with an example embodiment;

FIG. 11B illustrates a graph of intensity of fluorescence over time in accordance with an example embodiment;

FIG. 11C illustrates a graph of intensity of fluorescence over time in accordance with an example embodiment;

FIG. 11D illustrates a graph of intensity of fluorescence over time in accordance with an example embodiment;

FIG. 11E illustrates a graph of intensity of fluorescence over time in accordance with an example embodiment;

FIG. 11F illustrates a graph of intensity of fluorescence over time in accordance with an example embodiment;

FIG. 11G illustrates a graph of intensity of fluorescence over time in accordance with an example embodiment;

FIG. 12 illustrates a graph of intensity of fluorescence over time in accordance with an example embodiment;

FIG. 13A illustrates a graph of intensity of fluorescence over time in accordance with an example embodiment;

FIG. 13B illustrates a graph of intensity of fluorescence over time in accordance with an example embodiment;

FIG. 13C illustrates a graph of intensity of fluorescence over time in accordance with an example embodiment;

FIG. 13D illustrates a graph of intensity of fluorescence over time in accordance with an example embodiment;

FIG. 13E illustrates a graph of intensity of fluorescence over time in accordance with an example embodiment;

FIG. 13F illustrates a graph of intensity of fluorescence over time in accordance with an example embodiment;

FIG. 13G illustrates a graph of intensity of fluorescence over time in accordance with an example embodiment;

FIG. 14A illustrates a graph of intensity of fluorescence over time in accordance with an example embodiment;

FIG. 14B illustrates a graph of intensity of fluorescence over time in accordance with an example embodiment;

FIG. 14C illustrates a graph of intensity of fluorescence over time in accordance with an example embodiment;

FIG. 14D illustrates a graph of intensity of fluorescence over time in accordance with an example embodiment;

FIG. 14E illustrates a graph of intensity of fluorescence over time in accordance with an example embodiment;

FIG. 14F illustrates a graph of intensity of fluorescence over time in accordance with an example embodiment; and

FIG. 14G illustrates a graph of intensity of fluorescence over time in accordance with an example embodiment.

Reference will now be made to the exemplary embodiments illustrated, and specific language will be used herein to describe the same. It will nevertheless be understood that no limitation of the scope of the technology is thereby intended.

### DETAILED DESCRIPTION

Before invention embodiments are described, it is to be understood that this disclosure is not limited to the particular structures, process steps, or materials disclosed herein, but is extended to equivalents thereof as would be recognized by those ordinarily skilled in the relevant arts. It should also be understood that terminology employed herein is used for the purpose of describing particular examples or embodiments only and is not intended to be limiting. The same reference numerals in different drawings represent the same element. Numbers provided in flow charts and processes are provided for clarity in illustrating steps and operations and do not necessarily indicate a particular order or sequence.

Furthermore, the described features, structures, or characteristics can be combined in any suitable manner in one or more embodiments. In the following description, numerous specific details are provided, such as examples of compositions, storage, administration etc., to provide a thorough understanding of various invention embodiments. One skilled in the relevant art will recognize, however, that such detailed embodiments do not limit the overall inventive concepts articulated herein, but are merely representative thereof.

### Definitions

It should be noted that as used herein, the singular forms “a,” “an,” and, “the” include plural referents unless the context clearly dictates otherwise. Thus, for example, reference to “an excipient” includes reference to one or more of such excipients, and reference to “the carrier” includes reference to one or more of such carriers.

As used herein, the terms “formulation” and “composition” are used interchangeably and refer to a mixture of two or more compounds, elements, or molecules. In some aspects, the terms “formulation” and “composition” may be used to refer to a mixture of one or more active agents with a carrier or other excipients.

As used herein, the term “soluble” is a measure or characteristic of a substance or agent with regards to its ability to dissolve in a given solvent. The solubility of a substance or agent in a particular component of the composition refers to the amount of the substance or agent dissolved to form a visibly clear solution at a specified temperature such as about 25° C. or about 37° C.

As used herein, a “subject” refers to an animal. In one aspect the animal may be a mammal. In another aspect, the mammal may be a human.

As used herein, “non-liquid” when used to refer to the state of a composition disclosed herein refers to the physical state of the composition as being a semi-solid or solid. In this written description, the use of the term “solid” shall provide express support for the term “semisolid” and vice versa.

As used herein, “solid” and “semi-solid” refers to the physical state of a composition that supports its own weight at standard temperature and pressure and has adequate viscosity or structure to not freely flow. Semi-solid materials may conform to the shape of a container under applied pressure.

As used herein, a “solid phase medium” refers to a non-liquid medium. In one example, the non-liquid medium can be a material with a porous surface. In another example, the non-liquid medium can be a material with a fibrous surface. In yet another example, the non-liquid medium can be paper.

As used herein, a first nucleotide sequence can be joined to a second nucleotide sequence by a “linking sequence” when the first nucleotide sequence is coupled to a first end (e.g., 5′ or 3′ end) of the linking sequence and the second nucleotide sequence is coupled to a second end (e.g., 5′ or 3′ end) of the linking sequence. In one example, the first nucleotide sequence can be directly coupled to a first end of the linking sequence and the second nucleotide can be directly coupled to the second end of the linking sequence.

As used herein, a “forward inner primer (FIP)” can be a combination of an F1c primer and an F2 primer.

As used herein, an “F1c,” “F2,” “backward inner primer (BIP),” “B1c,” “B2,” “forward outer primer (F3),” “backward outer primer (B3),” “forward loop primer (LF),” “backward loop primer (LB),” refer to various primers used in an RT-LAMP reaction. These terms are well known in the art and their accepted meaning is intended herein.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” in the written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, comparative terms such as “increased,” “decreased,” “better,” “worse,” “higher,” “lower,” “enhanced,” “maximized,” “minimized,” and the like refer to a property of a device, component, composition, or activity that is measurably different from other devices, components, compositions or activities that are in a surrounding or adjacent area, that are similarly situated, that are in a single device or composition or in multiple comparable devices or compositions, that are in a group or class, that are in multiple groups or classes, or as compared to the known state of the art.

The term “coupled,” as used herein, is defined as directly or indirectly connected in a chemical, mechanical, electrical or nonelectrical manner. Objects described herein as being “adjacent to” each other may be in physical contact with each other, in close proximity to each other, or in the same general region or area as each other, as appropriate for the context in which the phrase is used. “Directly coupled” refers to objects, components, or structures that are in physical contact with one another and attached.

Occurrences of the phrase “in one embodiment,” or “in one aspect,” herein do not necessarily all refer to the same embodiment or aspect.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 angstroms to about 80 angstroms” should also be understood to provide support for the range of “50 angstroms to 80 angstroms.” Furthermore, it is to be understood that in this specification support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, levels and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges or decimal units encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

### Example Embodiments

An initial overview of invention embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

Selecting primer sets for loop-mediated isothermal amplification (LAMP) and reverse transcription LAMP (RT-LAMP) can be difficult because of the various constraints involved. First, the primer should have adequate stability to allow the LAMP reaction to proceed in a timely manner. Second, when used as a diagnostic test for a specific pathogen, the primer should target a unique sequence have minimal overlap with other potential pathogens, commensals, or background genome. Third, the limit of detection of a target pathogen should be low enough to allow detection of the target pathogen at low concentrations. Fourth, the false positive and false negative rates should be controlled to allow a reliability and a significant degree of confidence in the test results. Fifth, when conducting LAMP reactions on a solid-reaction medium (e.g. paper) slight defects which may not be an issue in liquid LAMP may pose an issue. Finally, in some cases, the reaction speed in a solid-based medium can be more than twice as slow as the reaction speed in a liquid-based medium.

A generalized approach to primer selection can rely on selected properties of the primers. For example, the guanine and cytosine (GC) content of a primer can provide a rough and ready way to approximate the stability of a primer. However, the GC content of a primer and related tools, can be misleading. As such, finding a specific primer sequence in a genome of tens of thousands of nucleotides can involve an extreme amount of experimentation. The amount of experimentation can be significantly controlled by using a process that uses a selected combination of primer parameters (e.g., nucleotide region length, length of primers, distance between primers, end stabilities, melting temperatures, minimum primer dimerization energy, distance between loop primers and inner primers, and screening based on reaction speed, limit of detection, and reducing false positives).

The nucleotide sequences resulting from such a process can have performance properties (e.g., low false positives, fast reaction speed, and low limit of detection). One of the primer sets identified based on this process is the RegX3.1 primer set, as identified herein. In one embodiment, the RegX3.1 primer set can include ten primers as follows: an F1c primer, an F2 primer, a B1c primer, a B2 primer, an F3 primer, a B3 primer, an LF primer, an LB primer, an FIP primer, and a BIP primer that can be associated with 10 distinct nucleotide sequences.

For example, in one disclosure embodiment, an isolated complementary DNA (cDNA) of a nucleic acid molecule can include a nucleotide sequence that is at least 85% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or a combination thereof.

In another disclosure embodiment, a primer set for reverse transcription loop-mediated isothermal amplification (RT-LAMP) analysis can include: a forward inner primer (FIP) sequence that is at least 85% identical to a combination of SEQ ID NO: 1 and SEQ ID NO: 2; a backward inner primer (BIP) sequence that is at least 85% identical to a combination of SEQ ID NO: 3 and SEQ ID NO: 4; a forward outer primer (F3) sequence that is at least 85% identical to SEQ ID NO: 5; a backward outer primer (B3) sequence that is at least 85% identical to SEQ ID NO: 6; a forward loop primer (LF) sequence that is at least 85% identical to SEQ ID NO: 7; and a backward loop primer (LB) sequence that is at least 85% identical to SEQ ID NO: 8. In some embodiments, the combination of SEQ ID NO: 1 and SEQ ID NO: 2 can be SEQ ID NO: 9. In other embodiments, the combination of SEQ ID NO: 3 and SEQ ID NO: 4 can be SEQ ID NO:10.

In yet another disclosure embodiment, a method of detecting a target pathogen from a Sarbecovirus in a subject can include providing a primer set. In one aspect, the primer set can include: a forward inner primer (FIP) sequence that is at least 85% identical to a combination of SEQ ID NO: 1 and SEQ ID NO: 2; a backward inner primer (BIP) sequence that is at least 85% identical to a combination of seq ID NO: 3 and SEQ ID NO: 4; a forward outer primer (F3) sequence that is at least 85% identical to SEQ ID NO: 5; a backward outer primer (B3) sequence that is at least 85% identical to SEQ ID NO: 6; a forward loop primer (LF) sequence that is at least 85% identical to SEQ ID NO: 7; and a backward loop primer (LB) sequence that is at least 85% identical to SEQ ID NO: 8. In some embodiments, the combination of SEQ ID NO: 1 and SEQ ID NO: 2 can be SEQ ID NO: 9. In other embodiments, the combination of SEQ ID NO: 3 and SEQ ID NO: 4 can be SEQ ID NO:10.

With the above-described background in mind, the present disclosure is drawn to cDNA, primer sets, and methods for reverse transcription loop-mediated isothermal amplification (RT-LAMP) analysis. The present disclosure is also drawn to detecting a target pathogen from a Sarbecovirus subgenus in a subject. The present disclosure is also drawn to various primer sets for reverse transcription loop-mediated isothermal amplification (RT-LAMP) analysis.

In one disclosure embodiment, an isolated complementary DNA (cDNA) of a nucleic acid molecule can have a specific nucleotide sequence. In one aspect, the nucleotide sequence can be at least 85% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, the like, or a combination thereof. In another aspect, the nucleotide sequence can be at least 90% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, the like, or a combination thereof. In another aspect, the nucleotide sequence can be at least 95% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, the like, or a combination thereof. In another aspect, the nucleotide sequence can be 100% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, the like, or a combination thereof.

In one example, the nucleotide sequence can be identical to SEQ ID NO: 9. In one aspect, SEQ ID NO: 9 can be a combination of SEQ ID NO: 1 and SEQ ID NO: 2. In one example, SEQ ID NO: 9 can be a combination of SEQ ID NO: 1 and SEQ ID NO: 2 when SEQ ID NO: 9 is 100% identical to a concatenation of SEQ ID NO: 1 and SEQ ID NO: 2 (e.g., SEQ ID: 1 is joined to SEQ ID NO: 2 without any intervening sequences between SEQ ID: 1 and SEQ ID: 2).

In one aspect, the nucleotide sequence can be at least 85% identical to SEQ ID NO: 9. In another aspect, the nucleotide sequence can be at least 90% identical to SEQ ID NO: 9. In yet another aspect, the nucleotide sequence can be at least 95% identical to SEQ ID NO: 9. In yet another aspect, the nucleotide sequence can be 100% identical to SEQ ID NO: 9.

In another aspect, the nucleotide sequence can include SEQ ID NO: 1 joined to SEQ ID NO: 2 by a linking sequence selected from Table 11. In this example, the linking sequence can be an intervening sequence between SEQ ID NO: 1 and SEQ ID NO: 2 without any other sequences between SEQ ID NO: 1 and SEQ ID NO: 2. In this example, when the linking sequence between SEQ ID NO: 1 and SEQ ID NO: 2 is removed, then the resulting sequence can be 85% identical, 90% identical, 95% identical, or 100% identical to SEQ ID NO: 9.

In one example, the nucleotide sequence can be identical to SEQ ID NO: 10. In one aspect, SEQ ID NO: 10 can be a combination of SEQ ID NO: 3 and SEQ ID NO: 4. In one example, SEQ ID NO: 10 can be a combination of SEQ ID NO: 3 and SEQ ID NO: 3 when SEQ ID NO: 10 is 100% identical to a concatenation of SEQ ID NO: 3 and SEQ ID NO: 3 (e.g., SEQ ID: 3 is joined to SEQ ID NO: 4 without any intervening sequences between SEQ ID: 3 and SEQ ID: 4).

In one aspect, the nucleotide sequence can be at least 85% identical to SEQ ID NO: 10. In another aspect, the nucleotide sequence can be at least 90% identical to SEQ ID NO: 10. In yet another aspect, the nucleotide sequence can be at least 95% identical to SEQ ID NO: 10. In yet another aspect, the nucleotide sequence can be 100% identical to SEQ ID NO: 10.

In another aspect, the nucleotide sequence can include SEQ ID NO: 3 joined to SEQ ID NO: 4 by a linking sequence selected from Table 11. In this example, the linking sequence can be an intervening sequence between SEQ ID NO: 3 and SEQ ID NO: 4 without any other sequences between SEQ ID NO: 3 and SEQ ID NO: 4. In this example, when the linking sequence between SEQ ID NO: 3 and SEQ ID NO: 4 is removed, then the resulting sequence can be 85% identical, 90% identical, 95% identical, or 100% identical to SEQ ID NO: 10.

In some cases, the thermodynamic parameters and other properties of the nucleotide sequence can impact the stability and performance of the RT-LAMP reaction. As depicted in Table 1, the thermodynamic parameters of the F3, B3, FIP, BIP, LF, LB, F2, F1c, B2, and B1c primers can fall within a selected range.

The 10 primers included in the orf7ab.1 (e.g., RegX3.1) primer set have relatively low guanine/cytosine (GC) content (30%-50%), with the average being about 39% GC for this primer set. Typically, a GC content between 45% and 65% can be achieved for many primer sets. As the GC content decreases below the range of 45% to 65%, decreasing stability is expected. However, that is not the case with the orf7ab.1 primer set because the end stabilities (the free energy change upon the binding of the last 6 base pairs on either the 5′ end of the 3′ end of the primer) are more negative than −2.5 kcal/mol for the orf7ab.1 primer set (with the exception of the 5′ end of LB and the 3′ ends of F2 and B2). This increased end stability relative to the −4.0-threshold combined with the lower GC content relative to a random sample of nucleotides may provide increased stability and performance for the orf7ab.1 primer set.

TABLE 1
thermodynamic parameters for primer set orf7ab.1

| | | Melting | 5' | 3' | |
|---|---|---|---|---|---|
| | | Temp | Stability | Stability | GC |
| Name | Length | (C) | (kcal/mol) | (kcal/mol) | Content |
| SARS-CoV- | 18 | 56.38 | −4.76 | −7.85 | 0.5 |
| 2_RegX3.1_F3 | | | | | |
| SARS-CoV- | 23 | 55.96 | −4.36 | −4.09 | 0.35 |
| 2_RegX3.1_B3 | | | | | |
| SARS-CoV- | 43 | | | | |
| 2_RegX3.1_FIP | | | | | |
| SARS-CoV- | 47 | | | | |
| 2_RegX3.1_BIP | | | | | |
| SARS-CoV- | 25 | 60.43 | −4.18 | −4.91 | 0.36 |
| 2_RegX3.1_LF | | | | | |
| SARS-CoV- | 20 | 55.15 | −3.52 | −4.91 | 0.35 |
| 2_RegX3.1_LB | | | | | |
| SARS-CoV- | 18 | 55.48 | −4.25 | −3.73 | 0.5 |
| 2_RegX3.1_F2 | | | | | |
| SARS-CoV- | 25 | 60.24 | −4.69 | −5.04 | 0.4 |
| 2_RegX3.1_F1C | | | | | |
| SARS-CoV- | 23 | 55.98 | −4.74 | −3.57 | 0.3 |
| 2_RegX3.1_B2 | | | | | |
| SARS-CoV- | 24 | 60.75 | −4.55 | −7.93 | 0.38 |
| 2_RegX3.1_BIC | | | | | |

In one aspect, the guanine and cytosine (GC) content of the nucleotide sequence can be less than or equal to a selected percentage. The selected percentage of GC content can be based on an end stability of the nucleotide sequence. In one example, the GC content of the nucleotide sequence can be 50% or less (e.g., less than or equal to 50% of the nucleotide sequence are comprised of guanine (G) or cytosine (C), with the remaining nucleotides of the nucleotide sequence being comprised of adenine (A) or thymine (T)). In one example, the GC content of the nucleotide sequence can be 45% or less. In another example, the GC content of the nucleotide sequence can be 40% or less. In yet another example, the GC content of the nucleotide sequence can be 35% or less.

In another aspect, at least one end stability of the nucleotide sequence (e.g., the 5′ end, the 3′ end, or both the 5′ end and the 3′ end of the nucleotide sequence) can have a stability that is less than or equal to a selected stability number. The selected stability number can be based on one or more of: the selected percentage of GC content, the selected temperature range, the like, or combinations thereof. In one example, the at least one end stability of the nucleotide sequence can be less than −2.5 kcal/mol (i.e., more negative). In another example, the at least one end stability of the nucleotide sequence can be less than −5.0 kcal/mol. In another example, the at least one end stability of the nucleotide sequence can be less than −6.0 kcal/mol. In another example, the at least one end stability of the nucleotide sequence can be less than −7.0 kcal/mol. In one aspect, both the 5′ end and the 3′ end of the nucleotide sequence can be less than at least one of −2.5 kcal/mol, −4.0 kcal/mol, −5.0 kcal/mol, −6.0 kcal/mol, −7.0 kcal/mol, the like, or combinations thereof.

In another aspect, the nucleotide sequence can have a melting temperature within a selected temperature range. The selected temperature range can be based on one or more of: the temperature range for activation of a reverse transcriptase, a temperature range for a DNA polymerase, the like, or a combination thereof. In one example, the nucleotide sequence can have a melting temperature of from about 40° C. to about 62° C. In another example, the nucleotide sequence can have a melting temperature of from about 50° C. to about 62° C. In one example, the nucleotide sequence can have a melting temperature of from about 55° C. to about 62° C.

In yet another aspect, the nucleotide sequence can have a selected minimum primer dimerization energy. The selected minimum primer dimerization energy can be based on one or more of: the selected percentage of GC content, the selected stability number, the selected temperature range, the like, or combinations thereof. In one example, the minimum primer dimerization energy can be less than −0.5 kcal/mol. In another example, the minimum primer dimerization energy can be less than −1.0 kcal/mol. In another example, the minimum primer dimerization energy can be less than −2.5 kcal/mol. In yet another example, the minimum primer dimerization energy can be less than −5.0 kcal/mol.

In yet another aspect, the nucleotide sequence can have a cross-contamination homology that can be less than a cross-contamination percentage. In one example, the nucleotide sequence can be less 50% identical to nucleotide sequences associated with non-target agents (commensal microorganisms, other pathogens, and human genome). In one example, the nucleotide sequence can be less 40% identical to nucleotide sequences associated with non-target agents (commensal microorganisms, other pathogens, and human genome). In one example, the nucleotide sequence can be less 30% identical to nucleotide sequences associated with non-target agents (commensal microorganisms, other pathogens, and human genome). In one example, the nucleotide sequence can be less 20% identical to nucleotide sequences associated with non-target agents (commensal microorganisms, other pathogens, and human genome). In one example, the nucleotide sequence can be less 10% identical to nucleotide sequences associated with non-target agents (commensal microorganisms, other pathogens, and human genome).

In some disclosure embodiments, a primer set for RT-LAMP analysis can include: a forward inner primer (FIP) sequence that is at least 85% identical to a combination of SEQ ID NO: 1 and SEQ ID NO: 2; a backward inner primer (BIP) sequence that is at least 85% identical to a combination of seq ID NO: 3 and SEQ ID NO: 4; a forward outer primer (F3) sequence that is at least 85% identical to SEQ ID NO: 5; a backward outer primer (B3) sequence that is at least 85% identical to SEQ ID NO: 6; a forward loop primer (LF) sequence that is at least 85% identical to SEQ ID NO: 7; a backward loop primer (LB) sequence that is at least 85% identical to SEQ ID NO: 8, the like, or combinations thereof. In some embodiments, the combination of SEQ ID NO: 1 and SEQ ID NO: 2 can be SEQ ID NO: 9. In other embodiments, the combination of SEQ ID NO: 3 and SEQ ID NO: 4 can be SEQ ID NO:10.

The forward inner primer (FIP) and the backward inner primer (BIP) can be generated by combining two primers (e.g., the F1c and the F2 primers for the FIP primer, or the B1c and the B2 primers for the BIP primer). The F1c, F2, B1c, and B2 sequences can have linker sequences (L) such that the FIP primer can be F1c-L-F2 and the BIP primer can be B1c-L-B2. Table 11 contains a list of the F1c, F2, B1c, and B2 sub-primers that were used when generating the FIP and BIP primers.

In one example, the FIP sequence can be at least 90% identical to a combination of SEQ ID NO: 1 and SEQ ID NO: 2. In another example, the FIP sequence can be at least 95% identical to a combination of SEQ ID NO: 1 and SEQ ID NO: 2. In yet example, the FIP sequence can be 100% identical to a combination of SEQ ID NO: 1 and SEQ ID NO: 2, which is equivalent to SEQ ID NO: 9.

In one aspect, the FIP sequence can include a linking sequence joining SEQ ID NO: 1 and SEQ ID NO: 2. Regardless of the percentage homology between the FIP sequence and the combination of SEQ ID NO: 1 and SEQ ID NO: 2, the linking sequence between SEQ ID NO: 1 and SEQ ID NO: 2 can be a linking sequence that is selected from Table 11. In one example, the linking sequence joining SEQ ID NO: 1 and SEQ ID NO: 2 can be 85%, 90%, 95%, 100%, the like, or a combination thereof, identical to the linking sequence that is selected from Table 11.

In one example, the BIP sequence can be at least 90% identical to a combination of SEQ ID NO: 3 and SEQ ID NO: 4. In another example, the BIP sequence can be at least 95% identical to a combination of SEQ ID NO: 3 and SEQ ID NO: 4. In yet example, the BIP sequence can be 100% identical to a combination of SEQ ID NO: 3 and SEQ ID NO: 4, which is equivalent to SEQ ID NO: 10.

In one aspect, the BIP sequence can include a linking sequence joining SEQ ID NO: 3 and SEQ ID NO: 4. Regardless of the percentage homology between the BIP sequence and the combination of SEQ ID NO: 3 and SEQ ID NO: 4, the linking sequence between SEQ ID NO: 3 and SEQ ID NO: 4 can be a linking sequence that is selected from Table 11. In one example, the linking sequence joining SEQ ID NO: 3 and SEQ ID NO: 4 can be 85%, 90%, 95%, 100%, the like, or a combination thereof, identical to the linking sequence that is selected from Table 11.

The homology percentage between F3 and SEQ ID NO: 5 can vary within a selected percentage range. In one example, the F3 sequence can be at least 90% identical to SEQ ID NO: 5. In another aspect, the F3 sequence can be at least 95% identical to SEQ ID NO: 5. In another aspect, the F3 sequence can be 100% identical to SEQ ID NO: 5.

The homology percentage between B3 and SEQ ID NO: 6 can vary within a selected percentage range. In another aspect, the B3 sequence can be at least 90% identical to SEQ ID NO: 6. In another aspect, the B3 sequence can be at least 95% identical to SEQ ID NO: 6. In another aspect, the B3 sequence can be 100% identical to SEQ ID NO: 6.

The homology percentage between LF and SEQ ID NO: 7 can vary within a selected percentage range. In one aspect, the LF sequence can be at least 90% identical to SEQ ID NO: 7. In another aspect, the LF sequence can be at least 95% identical to SEQ ID NO: 7. In another aspect, the LF sequence can be 100% identical to SEQ ID NO: 7.

The homology percentage between LB and SEQ ID NO: 8 can vary within a selected percentage range. In another aspect, the LB sequence can be at least 90% identical to SEQ ID NO: 8. In another aspect, the LB sequence can be at least 95% identical to SEQ ID NO: 8. In another aspect, the LB sequence can be 100% identical to SEQ ID NO: 8.

In another aspect, the GC content of the FIP, the BIP, the F3, the B3, the LF, the LB, the like, or a combination thereof can be one or more of: 50% or less, 45% or less, 40% or less, 35% or less, the like, or a combination thereof.

In yet another aspect, an end stability of the FIP, the BIP, the F3, the B3, the LF, the LB, the like, or a combination thereof can be less than one or more of: −2.5 kcal/mol, −4.0 kcal/mol, −5.0 kcal/mol, −6.0 kcal/mol, −7.0 kcal/mol, the like, or a combination thereof.

In another aspect, the FIP, the BIP, the F3, the B3, the LF, the LB, the like, or a combination thereof can have a melting temperature in a temperature range of from: about 40° C. to about 62° C.; or about 50° C. to about 62° C.; or about 55° C. to about 62° C.

In yet another aspect, the FIP, the BIP, the F3, the B3, the LF, the LB, the like, or a combination thereof can have a minimum primer dimerization energy of less than one or more of: −0.5 kcal/mol, −1.0 kcal/mol, −2.0 kcal/mol, −4.0 kcal/mol, −5.0 kcal/mol, the like, or combinations thereof.

In another aspect, the FIP, the BIP, the F3, the B3, the LF, the LB, the like, or a combination thereof can be less identical to nucleotide sequences associated with non-target agents (commensal microorganisms, other pathogens, and human genome) than a selected percentage. In one example, the selected percentage can be less than or equal to one or more of: 50%, 40%, 30%, 20%, 10%, the like, or combinations thereof.

In another disclosure embodiment, a method of detecting a target pathogen in a subject can include providing a primer set. In one aspect, the primer set can include: a forward inner primer (FIP) sequence that is at least 85% identical to a combination of SEQ ID NO: 1 and SEQ ID NO: 2; a backward inner primer (BIP) sequence that is at least 85% identical to a combination of seq ID NO: 3 and SEQ ID NO: 4; a forward outer primer (F3) sequence that is at least 85% identical to SEQ ID NO: 5; a backward outer primer (B3) sequence that is at least 85% identical to SEQ ID NO: 6; a forward loop primer (LF) sequence that is at least 85% identical to SEQ ID NO: 7; a backward loop primer (LB) sequence that is at least 85% identical to SEQ ID NO: 8; the like, or combinations thereof.

The target pathogen can comprise various pathogen types. In one aspect, the pathogen target can be one or more of a viral pathogen, a bacterial pathogen, a fungal pathogen, a protozoa pathogen, the like, or combinations thereof. The pathogen target can be detected when the nucleic acid from the pathogen target can be released from a cell wall, a cell membrane, a protein coat, or the like.

More specifically, in one aspect, the pathogen target can be a viral target. In some aspects, the viral target can be H1N1, H2N2, H3N2, H1N1pdm09, severe acute respiratory syndrome coronavirus 1 (SARS-CoV-1), severe acute respiratory syndrome coronavirus 2 (SARS-CoV-2), Middle East respiratory syndrome (MERS), influenza, the like, or combinations thereof.

In one example, the target pathogen can be a human coronavirus selected from: Severe Acute Respiratory Syndrome (SARS)-CoV (SARS-CoV), Severe Acute Respiratory Syndrome (SARS)-CoV 2 (SARS-CoV-2), Middle East Respiratory Syndrome (MERS)-CoV (MERS-CoV), SARS-CoV hCoV-HKU1, hCoV-0C43, hCoV-NL63, and hCoV-229E. In one example, the subject can be a human subject. In yet another example, the target pathogen can be Severe Acute Respiratory Syndrome (SARS)-CoV 2 (SARS-CoV-2).

When the pathogen target includes RNA, the RNA can be reverse transcribed. Therefore, in another aspect, the LAMP detection can be reverse transcription RT-LAMP. In this example, cDNA can be generated from a target RNA with a reverse transcriptase enzyme. The cDNA can be amplified to a detectable amount. When the pathogen target can be detected directly from DNA, then LAMP can be used to amplify the DNA to a detectable amount without reverse transcribing the RNA to DNA.

### Additional Primer Sets

In another disclosure embodiment, a primer set for RT-LAMP analysis can include: (a) an FIP sequence that is at least 85% identical to a combination of SEQ ID NO: 11 and SEQ ID NO: 12; (b) a BIP sequence that is at least 85% identical to a combination of seq ID NO: 13 and SEQ ID NO: 14; (c) an F3 sequence that is at least 85% identical to SEQ ID NO: 15; (d) a B3 sequence that is at least 85% identical to SEQ ID NO: 16; (e) an LF sequence that is at least 85% identical to SEQ ID NO: 17; and (f) an LB sequence that is at least 85% identical to SEQ ID NO: 18. In one aspect, the FIP sequence can be 100% identical to a combination of SEQ ID NO: 11 and SEQ ID NO: 12, which can be equivalent to SEQ ID NO: 19. In another aspect, the FIP sequence can include a linking sequence joining SEQ ID NO: 11 and SEQ ID NO: 12, wherein the linking sequence is selected from Table 11. In another aspect, the BIP sequence can be 100% identical to a combination of SEQ ID NO: 13 and SEQ ID NO: 14, which can be equivalent to SEQ ID NO: 20. In another aspect, the BIP sequence can include a linking sequence joining SEQ ID NO: 13 and SEQ ID NO: 14, wherein the linking sequence is selected from Table 11.

In another disclosure embodiment, a primer set for RT-LAMP analysis can include: (a) an FIP sequence that is at least 85% identical to a combination of SEQ ID NO: 21 and SEQ ID NO: 22; (b) a BIP sequence that is at least 85% identical to a combination of seq ID NO: 23 and SEQ ID NO: 24; (c) an F3 sequence that is at least 85% identical to SEQ ID NO: 25; (d) a B3 sequence that is at least 85% identical to SEQ ID NO: 26; (e) an LF sequence that is at least 85% identical to SEQ ID NO: 27; and (f) an LB sequence that is at least 85% identical to SEQ ID NO: 28. In one aspect, the FIP sequence can be 100% identical to a combination of SEQ ID NO: 21 and SEQ ID NO: 22, which can be equivalent to SEQ ID NO: 29. In another aspect, the FIP sequence can include a linking sequence joining SEQ ID NO: 21 and SEQ ID NO: 22, wherein the linking sequence is selected from Table 11. In another aspect, the BIP sequence can be 100% identical to a combination of SEQ ID NO: 23 and SEQ ID NO: 24, which can be equivalent to SEQ ID NO: 30. In another aspect, the BIP sequence can include a linking sequence joining SEQ ID NO: 23 and SEQ ID NO: 24, wherein the linking sequence is selected from Table 11.

In another disclosure embodiment, a primer set for RT-LAMP analysis can include: (a) an FIP sequence that is at least 85% identical to a combination of SEQ ID NO: 31 and SEQ ID NO: 32; (b) a BIP sequence that is at least 85% identical to a combination of seq ID NO: 33 and SEQ ID NO: 34; (c) an F3 sequence that is at least 85% identical to SEQ ID NO: 35; (d) a B3 sequence that is at least 85% identical to SEQ ID NO: 36; (e) an LF sequence that is at least 85% identical to SEQ ID NO: 37; and (f) an LB sequence that is at least 85% identical to SEQ ID NO: 38. In one aspect, the FIP sequence can be 100% identical to a combination of SEQ ID NO: 31 and SEQ ID NO: 32, which can be equivalent to SEQ ID NO: 39. In another aspect, the FIP sequence can include a linking sequence joining SEQ ID NO: 31 and SEQ ID NO: 32, wherein the linking sequence is selected from Table 11. In another aspect, the BIP sequence can be 100% identical to a combination of SEQ ID NO: 33 and SEQ ID NO: 34, which can be equivalent to SEQ ID NO: 40. In another aspect, the BIP sequence can include a linking sequence joining SEQ ID NO: 33 and SEQ ID NO: 34, wherein the linking sequence is selected from Table 11.

In another disclosure embodiment, a primer set for RT-LAMP analysis can include: (a) an FIP sequence that is at least 85% identical to a combination of SEQ ID NO: 41 and SEQ ID NO: 42; (b) a BIP sequence that is at least 85% identical to a combination of seq ID NO: 43 and SEQ ID NO: 44; (c) an F3 sequence that is at least 85% identical to SEQ ID NO: 45; (d) a B3 sequence that is at least 85% identical to SEQ ID NO: 46; (e) an LF sequence that is at least 85% identical to SEQ ID NO: 47; and (f) an LB sequence that is at least 85% identical to SEQ ID NO: 48. In one aspect, the FIP sequence can be 100% identical to a combination of SEQ ID NO: 41 and SEQ ID NO: 42, which can be equivalent to SEQ ID NO: 49. In another aspect, the FIP sequence can include a linking sequence joining SEQ ID NO: 41 and SEQ ID NO: 42, wherein the linking sequence is selected from Table 11. In another aspect, the BIP sequence can be 100% identical to a combination of SEQ ID NO: 43 and SEQ ID NO: 44, which can be equivalent to SEQ ID NO: 50. In another aspect, the BIP sequence can include a linking sequence joining SEQ ID NO: 43 and SEQ ID NO: 44, wherein the linking sequence is selected from Table 11

In another disclosure embodiment, a primer set for RT-LAMP analysis can include: (a) an FIP sequence that is at least 85% identical to a combination of SEQ ID NO: 51 and SEQ ID NO: 52; (b) a BIP sequence that is at least 85% identical to a combination of seq ID NO: 53 and SEQ ID NO: 54; (c) an F3 sequence that is at least 85% identical to SEQ ID NO: 55; (d) a B3 sequence that is at least 85% identical to SEQ ID NO: 56; (e) an LF sequence that is at least 85% identical to SEQ ID NO: 57; and (f) an LB sequence that is at least 85% identical to SEQ ID NO: 58. In one aspect, the FIP sequence can be 100% identical to a combination of SEQ ID NO: 51 and SEQ ID NO: 52, which can be equivalent to SEQ ID NO: 59. In another aspect, the FIP sequence can include a linking sequence joining SEQ ID NO: 51 and SEQ ID NO: 52, wherein the linking sequence is selected from Table 11. In another aspect, the BIP sequence can be 100% identical to a combination of SEQ ID NO: 53 and SEQ ID NO: 54, which can be equivalent to SEQ ID NO: 60. In another aspect, the BIP sequence can include a linking sequence joining SEQ ID NO: 53 and SEQ ID NO: 54, wherein the linking sequence is selected from Table 11.

In another disclosure embodiment, a primer set for RT-LAMP analysis can include: (a) an FIP sequence that is at least 85% identical to a combination of SEQ ID NO: 61 and SEQ ID NO: 62; (b) a BIP sequence that is at least 85% identical to a combination of seq ID NO: 63 and SEQ ID NO: 64; (c) an F3 sequence that is at least 85% identical to SEQ ID NO: 65; (d) a B3 sequence that is at least 85% identical to SEQ ID NO: 66; (e) an LF sequence that is at least 85% identical to SEQ ID NO: 67; and (f) an LB sequence that is at least 85% identical to SEQ ID NO: 68. In one aspect, the FIP sequence can be 100% identical to a combination of SEQ ID NO: 61 and SEQ ID NO: 62, which can be equivalent to SEQ ID NO: 69. In another aspect, the FIP sequence can include a linking sequence joining SEQ ID NO: 61 and SEQ ID NO: 62, wherein the linking sequence is selected from Table 11. In another aspect, the BIP sequence can be 100% identical to a combination of SEQ ID NO: 63 and SEQ ID NO: 64, which can be equivalent to SEQ ID NO: 70. In another aspect, the BIP sequence can include a linking sequence joining SEQ ID NO: 63 and SEQ ID NO: 64, wherein the linking sequence is selected from Table 11.

### Nucleotide Sequences:

The primer sets that follow comprise: (1) an F1c primer, (2) an F2 primer, (3) a B1c primer, (4) a B2 primer, (5) an F3 primer, (6) a B3 primer, (7) an LF primer, (8) an LB primer, (9) an FIP primer, and (10) a BIP primer in that order for each primer set.

### REGX Nucleotide Sequences:

### REGX3.1 Primer Set

As used herein, the terms “REGX3.1” and “orf7ab.1” are used interchangeably and refer to the same primer set.

SEQ ID NO: 1 can be:
GGAGAGTAAAGTTCTTGAACTTCCT
SEQ ID NO: 2 can be:
AGTTACGTGCCAGATCAG
SEQ ID NO: 3 can be:
TGCGGCAATAGTGTTTATAACACT
SEQ ID NO: 4 can be:
ATGAAAGTTCAATCATTCTGTCT
SEQ ID NO: 5 can be:
CGGCGTAAAACACGTCTA
SEQ ID NO: 6 can be:
GCTAAAAAGCACAAATAGAAGTC
SEQ ID NO: 7 can be:
TGTCTGATGAACAGTTTAGGTGAAA
SEQ ID NO: 8 can be:
TTGCTTCACACTCAAAAGAA
SEQ ID NO: 9 can be:
GGAGAGTAAAGTTCTTGAACTTCCTAGTTACGTGCCAGATCAG
SEQ ID NO: 10 can be:
TGCGGCAATAGTGTTTATAACACTATGAAAGTTCAATCATTCTGTCT
REGX1.1 Primer Set
SEQ ID NO: 11 can be:
TTCCGTGTACCAAGCAATTTCATG
SEQ ID NO: 12 can be:
TGACACTAAGAGGGGTGTA
SEQ ID NO: 13 can be:
AAGAGCTATGAATTGCAGACACC
SEQ ID NO: 14 can be:
TGGACATTCCCCATTGAAG
SEQ ID NO: 15 can be:
GTCCGAACAACTGGACTT
SEQ ID NO: 16 can be:
GTCTTGATTATGGAATTTAAGGGAA
SEQ ID NO: 17 can be:
CTCATGTTCACGGCAGCAGTA
SEQ ID NO: 18 can be:
ATTGGCAAAGAAATTTGACAC
SEQ ID NO: 19 can be:
TTCCGTGTACCAAGCAATTTCATGTGACACTAAGAGGGGTGTA
SEQ ID NO: 20 can be:
AAGAGCTATGAATTGCAGACACCTGGACATTCCCCATTGAAG
REGX1.2 Primer Set
SEQ ID NO: 21 can be:
TTCCGTGTACCAAGCAATTTCATG
SEQ ID NO: 22 can be:
TGACACTAAGAGGGGTGTA
SEQ ID NO: 23 can be:
CTGAAAAGAGCTATGAATTGCAGAC
SEQ ID NO: 24 can be:
TTGGACATTCCCCATTGA
SEQ ID NO: 25 can be:
GTCCGAACAACTGGACTT
SEQ ID NO: 26 can be:
GTCTTGATTATGGAATTTAAGGGAA
SEQ ID NO: 27 can be:
TCATGTTCACGGCAGCAGTA
SEQ ID NO: 28 can be:
ATTGGCAAAGAAATTTGACACCT
SEQ ID NO: 29 can be:
TTCCGTGTACCAAGCAATTTCATGTGACACTAAGAGGGGTGTA
SEQ ID NO: 30 can be:
CTGAAAAGAGCTATGAATTGCAGACTTGGACATTCCCCATTGA
REGX2.1 Primer Set
SEQ ID NO: 31 can be:
AGCCGCATTAATCTTCAGTTCATC
SEQ ID NO: 32 can be:
TAAGCGTGTTGACTGGAC
SEQ ID NO: 33 can be:
AGAAAGGTTCAACACATGGTTGT
SEQ ID NO: 34 can be:
TAGGGTTACCAATGTCGTGA
SEQ ID NO: 35 can be:
CTGTCCACGAGTGCTTTG
SEQ ID NO: 36 can be:
TGAGGTACACACTTAATAGCTT
SEQ ID NO: 37 can be:
ACCAATTATAGGATATTCAAT
SEQ ID NO: 38 can be:
AGCAGACAAATTCCCAGTTCT
SEQ ID NO: 39 can be:
AGCCGCATTAATCTTCAGTTCATCTAAGCGTGTTGACTGGAC
SEQ ID NO: 40 can be:
AGAAAGGTTCAACACATGGTTGTTAGGGTTACCAATGTCGTGA
REGX2.2 Primer Set
SEQ ID NO: 41 can be:
GCCGCATTAATCTTCAGTTCATCA
SEQ ID NO: 42 can be:
TTAAGCGTGTTGACTGGA
SEQ ID NO: 43 can be:
AGAAAGGTTCAACACATGGTTGTTA
SEQ ID NO: 44 can be:
TTAGGGTTACCAATGTCGT
SEQ ID NO: 45 can be:
CTGTCCACGAGTGCTTTG
SEQ ID NO: 46 can be:
TGAGGTACACACTTAATAGCT
SEQ ID NO: 47 can be:
CCAATTATAGGATATTCAATAG
SEQ ID NO: 48 can be:
TGCATTATTAGCAGACAAATTCCCA
SEQ ID NO: 49 can be:
GCCGCATTAATCTTCAGTTCATCATTAAGCGTGTTGACTGGA
SEQ ID NO: 50 can be:
AGAAAGGTTCAACACATGGTTGTTATTAGGGTTACCAATGTCGT
REGX2.3 Primer Set
SEQ ID NO: 51 can be:
GCCGCATTAATCTTCAGTTCATCA
SEQ ID NO: 52 can be:
TTAAGCGTGTTGACTGGA
SEQ ID NO: 53 can be:
AGAAAGGTTCAACACATGGTTGTT
SEQ ID NO: 54 can be:
TTAGGGTTACCAATGTCGT
SEQ ID NO: 55 can be:
CTGTCCACGAGTGCTTTG
SEQ ID NO: 56 can be:
TGAGGTACACACTTAATAGCT
SEQ ID NO: 57 can be:
CCAATTATAGGATATTCAATAG
SEQ ID NO: 58 can be:
TGCATTATTAGCAGACAAATTCCCA
SEQ ID NO: 59 can be:
GCCGCATTAATCTTCAGTTCATCATTAAGCGTGTTGACTGGA
SEQ ID NO: 60 can be:
AGAAAGGTTCAACACATGGTTGTTTTAGGGTTACCAATGTCGT
REGX2.4 Primer Set
SEQ ID NO: 61 can be:
GCCGCATTAATCTTCAGTTCATCA
SEQ ID NO: 62 can be:
TTAAGCGTGTTGACTGGAC
SEQ ID NO: 63 can be:
AGAAAGGTTCAACACATGGTTGTT
SEQ ID NO: 64 can be:
TTAGGGTTACCAATGTCGT
SEQ ID NO: 65 can be:
CTGTCCACGAGTGCTTTG
SEQ ID NO: 66 can be:
TGAGGTACACACTTAATAGCT
SEQ ID NO: 67 can be:
CCAATTATAGGATATTCAATA
SEQ ID NO: 68 can be:
TGCATTATTAGCAGACAAATTCCCA
SEQ ID NO: 69 can be:
GCCGCATTAATCTTCAGTTCATCATTAAGCGTGTTGACTGGAC
SEQ ID NO: 70 can be:
AGAAAGGTTCAACACATGGTTGTTTTAGGGTTACCAATGTCGT

### N Nucleotide Sequences:

N3 Primer Set
SEQ ID NO: 71 can be: CCACTGCGTTCTCCATTCTGGT
SEQ ID NO: 72 can be: AAATGCACCCCGCATTACG
SEQ ID NO: 73 can be: CGCGATCAAAACAACGTCGGC
SEQ ID NO: 74 can be: CCTTGCCATGTTGAGTGAGA
SEQ ID NO: 75 can be: TGGACCCCAAAATCAGCG
SEQ ID NO: 76 can be: GCCTTGTCCTCGAGGGAAT
SEQ ID NO: 77 can be: GTTGAATCTGAGGGTCCACCA
SEQ ID NO: 78 can be: ACCCAATAATACTGCGTCTTGG
SEQ ID NO: 79 can be:
CCACTGCGTTCTCCATTCTGGTAAATGCACCCCGCATTACG
SEQ ID NO: 80 can be:
CGCGATCAAAACAACGTCGGCCCTTGCCATGTTGAGTGAGA
N6 Primer Set
SEQ ID NO: 81 can be: CGACGTTGTTTTGATCGCGCC
SEQ ID NO: 82 can be: ATTACGTTTGGTGGACCCTC
SEQ ID NO: 83 can be: GCGTCTTGGTTCACCGCTCT
SEQ ID NO: 84 can be: AATTGGAACGCCTTGTCCTC
SEQ ID NO: 85 can be: CCCCAAAATCAGCGAAATGC
SEQ ID NO: 86 can be: AGCCAATTTGGTCATCTGGA
SEQ ID NO: 87 can be: TCCATTCTGGTTACTGCCAGTTG
SEQ ID NO: 88 can be: CAACATGGCAAGGAAGACCTT
SEQ ID NO: 89 can be:
CGACGTTGTTTTGATCGCGCCATTACGTTTGGTGGACCCTC
SEQ ID NO: 90 can be:
GCGTCTTGGTTCACCGCTCTAATTGGAACGCCTTGTCCTC
N10 Primer Set
SEQ ID NO: 91 can be: CGCCTTGTCCTCGAGGGAATT
SEQ ID NO: 92 can be: CGTCTTGGTTCACCGCTC
SEQ ID NO: 93 can be: AGACGAATTCGTGGTGGTGACG
SEQ ID NO: 94 can be: TGGCCCAGTTCCTAGGTAG
SEQ ID NO: 95 can be: GCCCCAAGGTTTACCCAAT
SEQ ID NO: 96 can be: AGCACCATAGGGAAGTCCAG
SEQ ID NO: 97 can be: TCTTCCTTGCCATGTTGAGTG
SEQ ID NO: 98 can be: ATGAAAGATCTCAGTCCAAGATGG
SEQ ID NO: 99 can be:
CGCCTTGTCCTCGAGGGAATTCGTCTTGGTTCACCGCTC
SEQ ID NO: 100 can be:
AGACGAATTCGTGGTGGTGACGTGGCCCAGTTCCTAGGTAG
N13e Primer Set
SEQ ID NO: 101 can be: GTCTTTGTTAGCACCATAGGGAAGTCC
SEQ ID NO: 102 can be: TGAAAGATCTCAGTCCAAGATGG
SEQ ID NO: 103 can be: GGAGCCTTGAATACACCAAAAGATCAC
SEQ ID NO: 104 can be: TTGAGGAAGTTGTAGCACGATTG
SEQ ID NO: 105 can be: AATTGGCTACTACCGAAGAGCTA
SEQ ID NO: 106 can be: GTAGAAGCCTTTTGGCAATGTTG
SEQ ID NO: 107 can be: TGGCCCAGTTCCTAGGTAGTAGAAATA
SEQ ID NO: 108 can be: CGCAATCCTGCTAACAATGCTG
SEQ ID NO: 109 can be:
GTCTTTGTTAGCACCATAGGGAAGTCCTGAAAGATCTCAGTCCAAGATGG
SEQ ID NO: 110 can be:
GGAGCCTTGAATACACCAAAAGATCACTTGAGGAAGTTGTAGCACGATTG

### Rdrp Nucleotide Sequences:

RdRp.1 Primer Set
SEQ ID NO: 111 can be: CAGTTGAAACTACAAATGGAACACC
SEQ ID NO: 112 can be: TACAGTGTTCCCACCTACA
SEQ ID NO: 113 can be: AGCTAGGTGTTGTACATAATCAGGA
SEQ ID NO: 114 can be: GGTCAGCAGCATACACAAG
SEQ ID NO: 115 can be: CAGATGCATTCTGCATTGT
SEQ ID NO: 116 can be: ATTACCAGAAGCAGCGTG
SEQ ID NO: 117 can be: TTTTCTCACTAGTGGTCCAAAACT
SEQ ID NO: 118 can be: TGTAAACTTACATAGCTCTAGACTT
SEQ ID NO: 119 can be:
CAGTTGAAACTACAAATGGAACACCTACAGTGTTCCCACCTACA
SEQ ID NO: 120 can be:
AGCTAGGTGTTGTACATAATCAGGAGGTCAGCAGCATACACAAG
RdRp.2 Primer Set
SEQ ID NO: 121 can be: GCCAACCACCATAGAATTTGCT
SEQ ID NO: 122 can be: AATAGCCGCCACTAGAGG
SEQ ID NO: 123 can be: AGTGATGTAGAAAACCCTCACCT
SEQ ID NO: 124 can be: AGGCATGGCTCTATCACAT
SEQ ID NO: 125 can be: ACTATGACCAATAGACAGTTTCA
SEQ ID NO: 126 can be: GGCCATAATTCTAAGCATGTT
SEQ ID NO: 127 can be: GTTCCAATTACTACAGTAGC
SEQ ID NO: 128 can be: ATGGGTTGGGATTATCCTAA
SEQ ID NO: 129 can be:
GCCAACCACCATAGAATTTGCTAATAGCCGCCACTAGAGG
SEQ ID NO: 130 can be:
AGTGATGTAGAAAACCCTCACCTAGGCATGGCTCTATCACAT
RdRp.3 Primer Set
SEQ ID NO: 131 can be: ATCACCCTGTTTAACTAGCATTGT
SEQ ID NO: 132 can be: TGACCTTACTAAAGGACCTC
SEQ ID NO: 133 can be: TATGTGTACCTTCCTTACCCAGA
SEQ ID NO: 134 can be: CCATCTGTTTTTACGATATCATCT
SEQ ID NO: 135 can be: GCAAAATGTTGGACTGAGAC
SEQ ID NO: 136 can be: GAACCGTTCAATCATAAGTGTA
SEQ ID NO: 137 can be: ATGTTGAGAGCAAAATTCAT
SEQ ID NO: 138 can be: TCCATCAAGAATCCTAGGGGC
SEQ ID NO: 139 can be:
ATCACCCTGTTTAACTAGCATTGTTGACCTTACTAAAGGACCTC
SEQ ID NO: 140 can be:
TATGTGTACCTTCCTTACCCAGACCATCTGTTTTTACGATATCATCT
RdRp.4 Primer Set
SEQ ID NO: 141 can be: ATGCGTAAAACTCATTCACAAAGTC
SEQ ID NO: 142 can be: CAACACAGACTTTATGAGTGTC
SEQ ID NO: 143 can be: TGATACTCTCTGACGATGCTGT
SEQ ID NO: 144 can be: AGCCACTAGACCTTGAGAT
SEQ ID NO: 145 can be: CGATAAGTATGTCCGCAATT
SEQ ID NO: 146 can be: ACTGACTTAAAGTTCTTTATGCT
SEQ ID NO: 147 can be: TGTGTCAACATCTCTATTTCTATAG
SEQ ID NO: 148 can be: TGTGTGTTTCAATAGCACTTATGC
SEQ ID NO: 149 can be:
ATGCGTAAAACTCATTCACAAAGTCCAACACAGACTTTATGAGTGTC
SEQ ID NO: 150 can be:
TGATACTCTCTGACGATGCTGTAGCCACTAGACCTTGAGAT

### Orflab Nucleotide Sequences:

Orf1ab.1 Primer Set
SEQ ID NO: 151 can be: TCCCCCACTAGCTAGATAATCTTTG
SEQ ID NO: 152 can be: CCAATTCAACTGTATTATCTTTCTG
SEQ ID NO: 153 can be: GTGTTAAGATGTTGTGTACACACAC
SEQ ID NO: 154 can be: ATCCATATTGGCTTCCGG
SEQ ID NO: 155 can be: AGCTGGTAATGCAACAGAA
SEQ ID NO: 156 can be: CACCACCAAAGGATTCTTG
SEQ ID NO: 157 can be: GCTTTAGCAGCATCTACAGCA
SEQ ID NO: 158 can be: TGGTACTGGTCAGGCAATAACAGT
SEQ ID NO: 159 can be:
TCCCCCACTAGCTAGATAATCTTTGCCAATTCAACTGTATTATCTTTCTG
SEQ ID NO: 160 can be:
GTGTTAAGATGTTGTGTACACACACATCCATATTGGCTTCCGG
Orf1ab.2 Primer Set
SEQ ID NO: 161 can be: TGACTGAAGCATGGGTTCGC
SEQ ID NO: 162 can be: GTCTGCGGTATGTGGAAAG
SEQ ID NO: 163 can be: GCTGATGCACAATCGTTTTTAAACG
SEQ ID NO: 164 can be: CATCAGTACTAGTGCCTGT
SEQ ID NO: 165 can be: ACTTAAAAACACAGTCTGTACC
SEQ ID NO: 166 can be: TCAAAAGCCCTGTATACGA
SEQ ID NO: 167 can be: GAGTTGATCACAACTACAGCCATA
SEQ ID NO: 168 can be: TTGCGGTGTAAGTGCAGCC
SEQ ID NO: 169 can be:
TGACTGAAGCATGGGTTCGCGTCTGCGGTATGTGGAAAG
SEQ ID NO: 170 can be:
GCTGATGCACAATCGTTTTTAAACGCATCAGTACTAGTGCCTGT
Orf1ab.3 Primer Set
SEQ ID NO: 171 can be: GATCACAACTACAGCCATAACCTTT
SEQ ID NO: 172 can be: GGGTTTTACACTTAAAAACACAG
SEQ ID NO: 173 can be: TGATGCACAATCGTTTTTAAACGG
SEQ ID NO: 174 can be: CATCAGTACTAGTGCCTGT
SEQ ID NO: 175 can be: TTGTGCTAATGACCCTGT
SEQ ID NO: 176 can be: TCAAAAGCCCTGTATACGA
SEQ ID NO: 177 can be: CCACATACCGCAGACGGTACAG
SEQ ID NO: 178 can be: GGTGTAAGTGCAGCCCGT
SEQ ID NO: 179 can be:
GATCACAACTACAGCCATAACCTTTGGGTTTTACACTTAAAAACACAG
SEQ ID NO: 180 can be:
TGATGCACAATCGTTTTTAAACGGCATCAGTACTAGTGCCTGT
Orf1ab.4 Primer Set
SEQ ID NO: 181 can be: ACAAGGTGGTTCCAGTTCTGTA
SEQ ID NO: 182 can be: GGGCTAGATTCCCTAAGAGT
SEQ ID NO: 183 can be: TGTTACAGACACACCTAAAGGTCC
SEQ ID NO: 184 can be: ACCATACCTCTATTTAGGTTGTT
SEQ ID NO: 185 can be: CTGTTATCCGATTTACAGGATT
SEQ ID NO: 186 can be: GGCAGCTAAACTACCAAGT
SEQ ID NO: 187 can be: TAGATAGTACCAGTTCCATC
SEQ ID NO: 188 can be: TGAAGTATTTATACTTTATTAAAGG
SEQ ID NO: 189 can be:
ACAAGGTGGTTCCAGTTCTGTAGGGCTAGATTCCCTAAGAGT
SEQ ID NO: 190 can be:
TGTTACAGACACACCTAAAGGTCCACCATACCTCTATTTAGGTTGTT

### E Nucleotide Sequences:

E.1 Primer Set
SEQ ID NO: 191 can be: CGTCGGTTCATCATAAATTGGTTC
SEQ ID NO: 192 can be: CACAATCGACGGTTCATCC
SEQ ID NO: 193 can be: ACTACTAGCGTGCCTTTGTAAGC
SEQ ID NO: 194 can be: GTCTCTTCCGAAACGAATG
SEQ ID NO: 195 can be: CCTGAAGAACATGTCCAAAT
SEQ ID NO: 196 can be: CGCTATTAACTATTAACGTACCT
SEQ ID NO: 197 can be: CATTACTGGATTAACAACTCC
SEQ ID NO: 198 can be: ACAAGCTGATGAGTACGAACTTATG
SEQ ID NO: 199 can be:
CGTCGGTTCATCATAAATTGGTTCCACAATCGACGGTTCATCC
SEQ ID NO: 200 can be:
ACTACTAGCGTGCCTTTGTAAGCGTCTCTTCCGAAACGAATG
E.2 Primer Set
SEQ ID NO: 201 can be: CGAAAGCAAGAAAAAGAAGTACGCT
SEQ ID NO: 202 can be: AGTACGAACTTATGTACTCATTCG
SEQ ID NO: 203 can be: TGGTATTCTTGCTAGTTACACTAGC
SEQ ID NO: 204 can be: AGACTCACGTTAACAATATTGC
SEQ ID NO: 205 can be: TTGTAAGCACAAGCTGATG
SEQ ID NO: 206 can be: AGAGTAAACGTAAAAAGAAGGTT
SEQ ID NO: 207 can be: ACGTACCTGTCTCTTCCGAAA
SEQ ID NO: 208 can be: CATCCTTACTGCGCTTCGATTGTG
SEQ ID NO: 209 can be:
CGAAAGCAAGAAAAAGAAGTACGCTAGTACGAACTTATGTACTCATTCG
SEQ ID NO: 210 can be:
TGGTATTCTTGCTAGTTACACTAGCAGACTCACGTTAACAATATTGC
E.3 Primer Set
SEQ ID NO: 211 can be: CTAGCAAGAATACCACGAAAGCAAG
SEQ ID NO: 212 can be: TTCGGAAGAGACAGGTACG
SEQ ID NO: 213 can be: CACTAGCCATCCTTACTGCGC
SEQ ID NO: 214 can be: AAGGTTTTACAAGACTCACGT
SEQ ID NO: 215 can be: GTACGAACTTATGTACTCATTCG
SEQ ID NO: 216 can be: TTTTTAACACGAGAGTAAACGT
SEQ ID NO: 217 can be: AGAAGTACGCTATTAACTATTA
SEQ ID NO: 218 can be: TTCGATTGTGTGCGTACTGCTG
SEQ ID NO: 219 can be:
CTAGCAAGAATACCACGAAAGCAAGTTCGGAAGAGACAGGTACG
SEQ ID NO: 220 can be:
CACTAGCCATCCTTACTGCGCAAGGTTTTACAAGACTCACGT
E.4 Primer Set
SEQ ID NO: 221 can be: ACGAGAGTAAACGTAAAAAGAAGGT
SEQ ID NO: 222 can be: GCTTCGATTGTGTGCGTA
SEQ ID NO: 223 can be: CTAGAGTTCCTGATCTTCTGGTCT
SEQ ID NO: 224 can be: TGGCTAAAATTAAAGTTCCAAAC
SEQ ID NO: 225 can be: CACTAGCCATCCTTACTGC
SEQ ID NO: 226 can be: GTACCGTTGGAATCTGCC
SEQ ID NO: 227 can be: AGACTCACGTTAACAATATTGCAGC
SEQ ID NO: 228 can be: ACGAACTAAATATTATATTAGTTTT
SEQ ID NO: 229 can be:
ACGAGAGTAAACGTAAAAAGAAGGTGCTTCGATTGTGTGCGTA
SEQ ID NO: 230 can be:
CTAGAGTTCCTGATCTTCTGGTCTTGGCTAAAATTAAAGTTCCAAAC
E.5 Primer Set
SEQ ID NO: 231 can be: CTGCCATGGCTAAAATTAAAGTTCC
SEQ ID NO: 232 can be: AGTTCCTGATCTTCTGGTCT
SEQ ID NO: 233 can be: TCCAACGGTACTATTACCGTTGA
SEQ ID NO: 234 can be: AAGGAATAGGAAACCTATTACTAGG
SEQ ID NO: 235 can be: ACTCTCGTGTTAAAAATCTGAA
SEQ ID NO: 236 can be: GCAAATTGTAGAAGACAAATCCAT
SEQ ID NO: 237 can be: AAAACTAATATAATATTTAGTTCGT
SEQ ID NO: 238 can be: AAAAAGCTCCTTGAACAATGGAA
SEQ ID NO: 239 can be:
CTGCCATGGCTAAAATTAAAGTTCCAGTTCCTGATCTTCTGGTCT
SEQ ID NO: 240 can be:
TCCAACGGTACTATTACCGTTGAAAGGAATAGGAAACCTATTACTAGG

### RNase P Nucleotide Sequences:

RNaseP.1 Primer Set
SEQ ID NO: 241 can be: GTTGCGGATCCGAGTCAGTGG
SEQ ID NO: 242 can be: CCGTGGAGCTTGTTGATGA
SEQ ID NO: 243 can be: AACTCAGCCATCCACATCCGAG
SEQ ID NO: 244 can be: TCACGGAGGGGATAAGTGG
SEQ ID NO: 245 can be: GGTGGCTGCCAATACCTC
SEQ ID NO: 246 can be: ACTCAGCATGCGAAGAGC
SEQ ID NO: 247 can be: GTGTGTCGGTCTCTGGCTCCA
SEQ ID NO: 248 can be: TCTTCAGGGTCACACCCAAGT
SEQ ID NO: 249 can be:
GTTGCGGATCCGAGTCAGTGGCCGTGGAGCTTGTTGATGA
SEQ ID NO: 250 can be:
AACTCAGCCATCCACATCCGAGTCACGGAGGGGATAAGTGG
RNaseP.2 Primer Set
SEQ ID NO: 251 can be: CGGATGTGGATGGCTGAGTTGT
SEQ ID NO: 252 can be: GAGCCAGAGACCGACACA
SEQ ID NO: 253 can be: ACTCCTCCACTTATCCCCTCCG
SEQ ID NO: 254 can be: TGGTCCGAGGTCCAGTAC
SEQ ID NO: 255 can be: CGTGGAGCTTGTTGATGAGC
SEQ ID NO: 256 can be: TGGGCTTCCAGGGAACAG
SEQ ID NO: 257 can be: ATCCGAGTCAGTGGCTCCCG
SEQ ID NO: 258 can be: ATATGGCTCTTCGCATGCTG
SEQ ID NO: 259 can be:
CGGATGTGGATGGCTGAGTTGTGAGCCAGAGACCGACACA
SEQ ID NO: 260 can be:
ACTCCTCCACTTATCCCCTCCGTGGTCCGAGGTCCAGTAC
RNaseP.3 Primer Set
SEQ ID NO: 261 can be: ACATGGCTCTGGTCCGAGGTC
SEQ ID NO: 262 can be: CTCCACTTATCCCCTCCGTG
SEQ ID NO: 263 can be: CTGTTCCCTGGAAGCCCAAAGG
SEQ ID NO: 264 can be: TAACTGGGCCCACCAAGAG
SEQ ID NO: 265 can be: TCAGGGTCACACCCAAGT
SEQ ID NO: 266 can be: CGCATACACACACTCAGGAA
SEQ ID NO: 267 can be: ACTCAGCATGCGAAGAGCCATAT
SEQ ID NO: 268 can be: CTGCATTGAGGGTGGGGGTAAT
SEQ ID NO: 269 can be:
ACATGGCTCTGGTCCGAGGTCCTCCACTTATCCCCTCCGTG
SEQ ID NO: 270 can be:
CTGTTCCCTGGAAGCCCAAAGGTAACTGGGCCCACCAAGAG
RNaseP.4 Primer Set
SEQ ID NO: 271 can be: CACTGGATCCAGTTCAGCCTCC
SEQ ID NO: 272 can be: GCACACAGCATGGCAGAA
SEQ ID NO: 273 can be: TTAGGAAAAGGCTTCCCAGCCG
SEQ ID NO: 274 can be: TGGGCCTTAAAGTCCGTCTT
SEQ ID NO: 275 can be: GCCCTGTGGAACGAAGAG
SEQ ID NO: 276 can be: TCCGTCCAGCAGCTTCTG
SEQ ID NO: 277 can be: CACCGCGGGGCTCTCGGT
SEQ ID NO: 278 can be: CTGCCCCGGAGACCCAATG
SEQ ID NO: 279 can be:
CACTGGATCCAGTTCAGCCTCCGCACACAGCATGGCAGAA
SEQ ID NO: 280 can be:
TTAGGAAAAGGCTTCCCAGCCGTGGGCCTTAAAGTCCGTCTT
RNaseP.5 Primer Set
SEQ ID NO: 281 can be: CACCTGCAAGGACCCGAAGC
SEQ ID NO: 282 can be: AACCGCGCCATCAACATC
SEQ ID NO: 283 can be: GCCAATACCTCCACCGTGGAG
SEQ ID NO: 284 can be: GTTGCGGATCCGAGTCAG
SEQ ID NO: 285 can be: TACATTCACGGCTTGGGC
SEQ ID NO: 286 can be: GGGTGTGACCCTGAAGACT
SEQ ID NO: 287 can be: CGCCTGCAGCTGCAGCGC
SEQ ID NO: 288 can be: GTTGATGAGCTGGAGCCAGAGA
SEQ ID NO: 289 can be:
CACCTGCAAGGACCCGAAGCAACCGCGCCATCAACATC
SEQ ID NO: 290 can be:
GCCAATACCTCCACCGTGGAGGTTGCGGATCCGAGTCAG

### EXAMPLES

The following examples are provided to promote a clearer understanding of certain embodiments of the present invention, and are in no way meant as a limitation thereon.

### Example 1—Primer Set Schematic

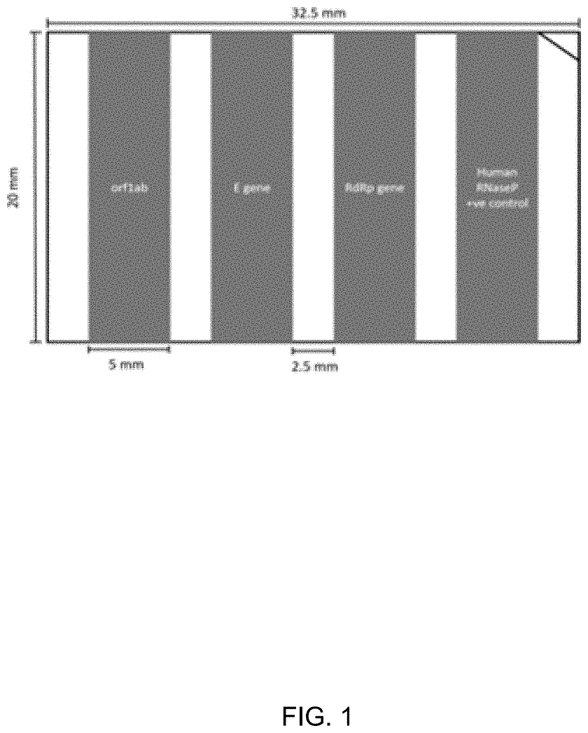

As illustrated in FIG. 1, the RNA from the SARS-CoV-2 virus in saliva was extracted, reverse-transcribed, and amplified in a one-pot mixture by heating the saliva and reagent mixture at 65° C. The four primer sets used for LAMP included: one targeting the SARS-CoV-2 RdRp gene, one targeting the SARS-CoV-2 envelope gene (E), one targeting the SARS-CoV-2 ORF lab region, and one targeting the human RNaseP (RP) gene which served as an on-board control.

The illustration in FIG. 1 represented the target RNA regions on the test paper in which the white spots represent spaces and the orange spots represent the test regions. Each orange test area was about 5 mm in width and 20 mm in height with about 2.5 mm between each orange test area. Each primer set was comprised of 6 individual primers—targeting specific regions of viral or human RNA which were reverse-transcribed and amplified during isothermal incubation using a reverse transcriptase and a strand-displacing polymerase. In this Example, a positive test interpretation was determined when a positive result in 2 of the 3 target gene primer regions of Orflab, E Gene or RdRp Gene was obtained.

### Example 2—Inclusivity Analysis

An in silico study was performed to characterize inclusivity and cross-reactivity of the LAMP assay primers. One assay included three primer sets: (a) targeting the E-gene (the envelope small membrane protein), (b) the RdRp gene (also known as the nsp12 gene which encodes viral polymerase), and (c) ORF lab region (encoding multiple non-structural proteins of clinical significance). Each primer set contained 6 primers. For both inclusivity and cross-reactivity studies, the BLASTn tool was used to align each primer sequence with the appropriate reference genomes.

The inclusivity study, as depicted in Table 2 shows the proportion of SARS-CoV-2 genomes that were detected by each primer set. Inclusivity was calculated by aligning each primer against 5332 SARS-CoV genome sequences downloaded from NCBI (txid2697049) on 12 Jun. 2020. A primer set was considered inclusive if all six primers in the set had 100% match for the target genome. The test employed 3 primer sets in which each set contained 6 individual primers. In addition, a positive SARS-CoV-2 test uses 2 of the 3 primer sets to show a positive reaction. Thus, the demonstrated 92-94% inclusivity across individual genes was an acceptable level for the test's individual gene components.

Table 2 in silico inclusivity analysis

| | E- | RdRp/nsp12 | |
|---|---|---|---|
| Primer Set | gene | gene | ORF1ab |
| total genomes | 5332 | 5332 | 5332 |
| perfect match | 5030 | 5020 | 4928 |
| mismatches = 1 | 70 | 59 | 43 |
| mismatches = 2 | 9 | 12 | 7 |
| mismatches = 3 | 4 | 5 | 3 |
| mismatches = 4 | 4 | 0 | 2 |
| mismatches >= 5 | 215 | 236 | 349 |
| % inclusivity | 94.3 | 94.1 | 92.4 |

Due to the large number of mutations SARS-CoV-2 has undergone, the primer sets exhibited mismatches of varying lengths for 5.7-7.6% of the tested strains. While the presence of a single mismatch within a target genome suggests a lack of inclusivity for that particular strain, this conclusion is not definitive. For example, previous work on MERS-CoV has demonstrated that a single nucleotide mismatch in one of the primers may not have an impact on the limit of detection of LAMP assays. Additionally, the LAMP reaction used 6 primers per set and two of them (e.g., the loop primers) were not used for amplification but rather contribute to the increase of the rate of the reaction. Successful amplification was possible even with mismatches in the loop primers. Therefore, the inclusivity percentages in Table 2 represent a worst-case assumption.

1n-silico inclusivity studies were then conducted to verify detection of SARS-CoV-2 with orflab.II primer set. RT-LAMP primers for orflab.II were aligned against publicly available SARS-CoV-2 whole genomes from the NCBI Nucleotide database as of Aug. 5, 2020. The orflab.II primer set had 100% sequence identity with 98.72% of the 8,844 sequences available; and 99.79% of the sequences contained 1 mismatch or less when aligned with the orflab.II primer set. The alignments which contained 2 or more mismatches (19 sequences) with the orflab.II primer set had multiple mismatches within an individual primer. Although the frequency of this occurrence was less than 0.5%, these types of mismatches had been shown to affect RT-LAMP reactions and could lead to false negatives.

Whole SARS-CoV-2 genomes were identified by filtering all SARS-CoV-2 genomes (as identified by the taxonomy ID #2697049) by: (i) genomic sequence type, (ii) inclusion of the phrase “whole genome” in the sequence name, and (iii) sequences between the lengths of 28,000 and 30,000 base pairs. This was performed by using the following Entrez query with the Entrez esearch utility to obtain the accession numbers: “txid2697049[Organism:noexp] AND (viruses[filter] AND biomol_genomic[PROP] AND (28000[SLEN]: 30000[SLEN])) AND (complete genome[All Fields]).” The Entrez efetch utility was used to download the complete FASTA sequences for each accession number. Primers were aligned to each sequence using the msa.sh (i.e., MultiStateAligner) function of BBMap v38.86. The CIGAR string contained in the resulting SAM file for each primer was used to determine the number of matches between the aligned primer and the subject sequence. Percent sequence identity was calculated using the number of matches divided by the alignment length (which was equal to the primer length for all cases). Inclusivity was determined by calculating the portion of SARS-CoV-2 whole genome sequences that had 100% sequence identity with all of the aligned primers. For a more flexible analysis, the number of mismatches was calculated for each primer alignment. For each sequence, if the sum of mismatches across all primers was less than a predetermined mismatch threshold, then the particular sequence was used for sequence inclusivity. For this analysis, the constituent primers of FIP (e.g., F1c and F2) and BIP (B1c/B2) were used in lieu of the FIP and BIP primers.

### Example 3—Cross-Reactivity Analysis

To predict cross-reactivity for each LAMP primer set, sequence similarity was calculated for each primer against a list of relevant off-target background genomes. The alignments were subsequently filtered for a ≥80% sequence match, as depicted in Table 3.

TABLE 3
in silico cross-reactivity analysis

| | PRIMERS WITH >80% | | |
|---|---|---|---|
| | SIMILARITY (#/6) | | |
| OFF-TARGET GENOME | E-gene | RdRp | ORF1ab |
| Human coronavirus 229E | 0 | 0 | 0 |
| Human coronavirus OC43 | 0 | 0 | 0 |
| Human coronavirus HKU1 | 0 | 0 | 0 |
| Human coronavirus NL63 | 0 | 0 | 0 |
| SARS | 6 | 6 | 6 |
| Middle East respiratory | 0 | 2 | 0 |
| syndrome-related coronavirus | | | |
| Chlamydia pneumoniae | 0 | 1 | 0 |
| Haemophilus influenzae | 1 | 1 | 0 |
| Legionella pneumophila | 0 | 0 | 0 |
| Mycobacterium tuberculosis | 0 | 0 | 0 |
| Streptococcus pneumoniae | 0 | 0 | 0 |
| Streptococcus pyogenes | 0 | 0 | 0 |
| Bordetella pertussis | 0 | 0 | 0 |
| Mycoplasma pneumoniae | 0 | 0 | 0 |
| Pneumocystis jirovecii | 0 | 0 | 0 |
| Pseudomonas aeruginosa | 0 | 1 | 0 |
| Staphylococcus epidermidis | 0 | 1 | 0 |
| Streptococcus salivarius | 0 | 0 | 0 |
| Adenovirus | 0 | 0 | 0 |
| Human metapneumovirus | 0 | 0 | 0 |
| Human parainfluenza virus | 0 | 0 | 1 |
| Influenza A | 0 | 1 | 0 |
| Influenza B | 0 | 0 | 0 |
| Enterovirus | 1 | 0 | 0 |
| Respiratory syncytial virus | 0 | 0 | 0 |
| Rhinovirus | 0 | 0 | 0 |
| Human GRCh38 | 2 | 2 | 2 |

Background genomes tested include those that were reasonably likely to be encountered in the clinical specimen. The primers were compared against the human reference genome (GRCh38.p13), and the nasal microbiome sequencing data (Accession: PRJNA342328) to represent diverse microbial flora in the human respiratory tract.

Results of the cross-reactivity analysis indicated a negligible chance of false-positives on off-target organisms. Columns in the table for each SARS-CoV-2 gene target indicated the number of primers in each set (out of six total) that scored above the 80% threshold. In a few cases (e.g., C. pneumoniae, H. influenzae), one primer in a set of six scored above the threshold. In this case, the risk of non-specific amplification was minimal because amplification cannot occur unless at least two primers bound the target. In the case of MERS, two primers out of six were highly similar to the RdRp gene. However, MERS is not prevalent in the United States, with 2 cases ever reported. Moreover, even if a false-positive for this marker were to occur, the lack of positive amplification on the other two markers would indicate a negative test result to the operator. The highest risk of cross-reactivity with off-target organisms appeared to be with related SARS viruses, especially human SARS-CoV-1, bat, and feline coronaviruses. Because SARS-CoV-1 is not currently extant in human populations, the chance of a false positive on this off-target can be considered negligible. Finally, two primers out of each set of six were similar to the human genome background. However, these primer sets have not exhibited non-specific amplification on human saliva specimens in experiments. These results indicate a low probability of false-positives due to cross-reactivity.

Additional wet lab testing can confirm these computational predictions using commercially-available panels (e.g., ZeptoMetrix Validation panels (#NATRVP-3, NATPPQ-BIO, NATPPA-BIO) with intact, inactivated organisms.

### Example 4A—In-Silico Identity Analysis

In-silico homology studies were also conducted against several potentially pathogenic microorganisms and viruses that can be found in the human saliva or in the human respiratory tract using BLAST. Organisms were found to be potentially cross-reactive if any primer was >80% identical as determined by percent identity. Consequently, four microorganisms were found to be potentially cross-reactive: SARS-coronavirus, Haemophilus influenzae, Pneumocystis jirovecii, and Pseudomonas aeruginosa. Both P. jirovecii and P. aeruginosa have one primer with >80% homology. As a result, the orflab.II primer set was not expected to be cross-reactive with these pathogens. Two primers were found to be potentially cross-reactive with H. influenzae; however, one of these two primers was a loop primer, which was primarily used to accelerate the RT-LAMP reaction. In the absence of more than one “core” primer (e.g., F3/B3 or FIP/BIP) being reactive, it was not expected that the orflab.II primer set would be cross-reactive with these organisms either. Four primers were found to be potentially cross-reactive with SARS-coronavirus; however, because of the low prevalence of this virus in general populations, there was minimal risk that orflab.II would produce false positives. Comprehensive results of the homology analysis can be found in Table 4A.

TABLE 4A
Results from the in-silico homology analysis for the orf1ab.II primer set.

| Taxon | TXID | F3 | B3 | FIP | BIP | LF | LB | Primers ≥ 0.8 |
|---|---|---|---|---|---|---|---|---|
| Human | 11137 | 0.59 | 0.63 | 0.28 | 0.30 | 0.50 | 0.58 | 0 |
| coronavirus 229E | | | | | | | | |
| Human | 31631 | 0.55 | 0.63 | 0.28 | 0.30 | 0.54 | 0.53 | 0 |
| coronavirus | | | | | | | | |
| OC43 | | | | | | | | |
| Human | 290028 | 0.50 | 0.47 | 0.26 | 0.27 | 0.54 | 0.47 | 0 |
| coronavirus | | | | | | | | |
| HKU1 | | | | | | | | |
| Human | 277944 | 0.50 | 0.47 | 0.31 | 0.30 | 0.50 | 0.63 | 0 |
| coronavirus | | | | | | | | |
| NL63 | | | | | | | | |
| SARS− | 694009 | 1.00 | 1.00 | 0.54 | 0.57 | 1.00 | 1.00 | 4 |
| coronavirus | | | | | | | | |
| MERS− | 1335626 | 0.64 | 0.79 | 0.28 | 0.32 | 0.67 | 0.63 | 0 |
| coronavirus | | | | | | | | |
| Human | 12730 | 0.73 | 0.58 | 0.26 | 0.27 | 0.46 | 0.58 | 0 |
| respirovirus 1 | | | | | | | | |
| Human | 1979160 | 0.45 | 0.58 | 0.26 | 0.27 | 0.54 | 0.74 | 0 |
| rubulavirus 2 | | | | | | | | |
| Human | 11216 | 0.64 | 0.47 | 0.33 | 0.34 | 0.50 | 0.58 | 0 |
| respirovirus 3 | | | | | | | | |
| Human | 1979161 | 0.68 | 0.47 | 0.23 | 0.30 | 0.54 | 0.53 | 0 |
| rubulavirus 4 | | | | | | | | |
| Influenza A | 11320 | 0.64 | 0.68 | 0.36 | 0.32 | 0.67 | 0.74 | 0 |
| Virus | | | | | | | | |
| Influenza B | 11520 | 0.45 | 0.58 | 0.28 | 0.25 | 0.46 | 0.58 | 0 |
| Virus | | | | | | | | |
| Human | 1193974 | 0.50 | 0.53 | 0.31 | 0.27 | 0.50 | 0.58 | 0 |
| Enterovirus | | | | | | | | |
| Human | 11250 | 0.55 | 0.53 | 0.28 | 0.27 | 0.50 | 0.53 | 0 |
| Respiratory | | | | | | | | |
| syncytial virus | | | | | | | | |
| Rhinovirus A | 147711 | 0.59 | 0.63 | 0.56 | 0.34 | 0.54 | 0.63 | 0 |
| Rhinovirus B | 147712 | 0.59 | 0.68 | 0.31 | 0.30 | 0.54 | 0.53 | 0 |
| Rhinovirus C | 463676 | 0.59 | 0.63 | 0.36 | 0.30 | 0.54 | 0.58 | 0 |
| Chlamydia | 83558 | 0.64 | 0.63 | 0.33 | 0.34 | 0.67 | 0.63 | 0 |
| pneumoniae | | | | | | | | |
| Haemophilus | 727 | 0.64 | 0.89 | 0.41 | 0.39 | 0.67 | 0.84 | 2 |
| influenzae | | | | | | | | |
| Legionella | 446 | 0.68 | 0.79 | 0.38 | 0.41 | 0.63 | 0.68 | 0 |
| pneumophila | | | | | | | | |
| Mycobacterium | 1773 | 0.00 | 0.58 | 0.41 | 0.00 | 0.00 | 0.74 | 0 |
| tuberculosis | | | | | | | | |
| Streptococcus | 1313 | 0.00 | 0.00 | 0.00 | 0.00 | 0.00 | 0.00 | 0 |
| pneumoniae | | | | | | | | |
| Streptococcus | 1314 | 0.68 | 0.74 | 0.33 | 0.43 | 0.58 | 0.74 | 0 |
| pyogenes | | | | | | | | |
| Bordetella | 520 | 0.55 | 0.63 | 0.41 | 0.00 | 0.00 | 0.63 | 0 |
| pertussis | | | | | | | | |
| Mycoplasma | 2104 | 0.68 | 0.53 | 0.33 | 0.39 | 0.58 | 0.68 | 0 |
| pneumoniae | | | | | | | | |
| Pneumocystis | 42068 | 0.73 | 0.84 | 0.33 | 0.43 | 0.67 | 0.74 | 1 |
| jirovecii | | | | | | | | |
| Candida albicans | 5476 | 0.64 | 0.68 | 0.36 | 0.48 | 0.67 | 0.79 | 0 |
| Pseudomonas | 287 | 0.59 | 0.84 | 0.44 | 0.39 | 0.63 | 0.79 | 1 |
| aeruginosa | | | | | | | | |
| Staphylococcus | 1282 | 0.64 | 0.74 | 0.41 | 0.43 | 0.58 | 0.68 | 0 |
| epidermis | | | | | | | | |
| Streptococcus | 1304 | 0.73 | 0.74 | 0.36 | 0.39 | 0.58 | 0.68 | 0 |
| salivarius | | | | | | | | |

In-silico homology analysis was conducted by performing a BLAST search of each primer against sequences available in the NCBI Nucleotide database for the specific taxon of interest. Parameters that were used in the BLAST search can be found in Table 4B (for the entrez query, “{TaxonID}” is replaced with the TaxonID of the respective microorganism). Sequence identity for each hit in the BLAST analysis was then calculated by using the number of matches for a hit divided by the length of the primer, not the alignment length. Homology was determined by calculating the maximum sequence identity of all hits for a specific primer against an individual organism and is reported in Table 4B. Primers with greater than 80% homology were deemed as potentially cross-reactive.

TABLE 4B

| Parameter | Value |
|---|---|
| Algorithm | blastn |
| Database | nt |
| Entrez Query | txid{TaxonID}[ORGN] |
| Expect threshold | 1000 |
| Alignments | 1000 |
| Match/Mistmatch Score | 1, −3 |
| Gap existence/extension | 5, 2 |

Interfering substances found in respiratory samples endogenously or exogenously can also be tested to evaluate the extent, if any, of potential assay inhibition. Bio-banked saliva specimens (e.g., frozen samples without preservative) can be spiked with 2× limit of detection (LoD) with inactivated virus to further characterize the potential assay inhibition.

### Example 4B—In-Silico Identity Analysis II

RT-LAMP primer sets were designed using PrimerExplorer v5 and are presented in Table 10. Parameters used to design primers can be found in Table 5A. All other Primer Explorer parameters were kept at their default values. Primer sets were designed using portions of the SARS-CoV-2 reference genome (NCBI accession number: NC 045512). Primer sets for RdRP were designed by first splitting the nsp12 gene sequence into 2 portions. Primer set RdRP.I was designed using the first portion of the nsp12 sequence, while primer sets RdRP.II and RdRP.III were designed using the second portion of the nsp12 sequence. Primer sets for orflab were designed using a portion of the orflab gene sequence. Primer sets for RegX were designed by choosing three random 2,000 nt regions of the reference genome. In-silico analyses were used to the predict sensitivity and specificity of each primer set. Optimal primer sets underwent experimental cross-reactivity studies to ensure specificity to SARS-CoV-2.

Whole SARS-CoV-2 genomes were identified by filtering all publicly available SARS-CoV-2 genomes from the NCBI Nucleotide database as of Feb. 5, 2021 (as identified by the taxon ID 2697049) by genomic sequence type, inclusion of the phrase “whole genome” in the sequence name, and sequences between the lengths of 28,000 and 30,000 base pairs. This identification was accomplished by using the following Entrez query with the Entrez esearch utility to obtain the accession numbers: “txid2697049[Organism:noexp] AND (viruses/filter] AND biomol_genomic[PROP] AND (28000[SLEN]: 30000[SLEN])) AND (complete genome[All Fields]).” The Entrez efetch utility was then used to download the complete FASTA sequences for each accession number. Primers were aligned to each sequence using the msa.sh (which stands for MultiStateAligner not Multiple Sequence Alignment) function of BBMap v38.86. The CIGAR string contained in the resulting SAM file for each primer was used to determine the number of matches between the aligned primer and the subject sequence. Percent sequence identity was calculated using the number of matches divided by the alignment length (which was equal to the primer length for all cases). Inclusivity was then determined by calculating the portion of SARS-CoV-2 whole genome sequences that had 100% sequence identity with all of the aligned primers. For a more relaxed analysis, the number of mismatches was calculated for each primer alignment. For each sequence, if the sum of mismatches across all primers was less than a given mismatch threshold (either 0, 1, or more), this sequence was counted for sequence inclusivity. For this analysis, the constituent primers of FIP and BIP, F1c/F2 and B1c/B2, respectively, were used in lieu of the FIP and BIP primers. The orflab.II and orf7ab.I primers set had 100% sequence identity with 97.52% and 95.12% of the 39,134 sequences available, respectively. When one mismatch was allowed across the entire set, the orflab.II and orf7ab.I primer sets then had 99.63% and 99.29% of the sequences meet this constraint.

We conducted in-silico inclusivity and sequence identity studies to verify the conservation of the RT-LAMP primers with available SARS-CoV-2 sequences and to predict cross-reactivity of our primer sets. In-silico sequence identity analyses were conducted by performing a BLAST search of each primer against sequences available in the NCBI Nucleotide database for the specific taxon of interest. Parameters that were used in the BLAST search can be found in Table 5B. The sequence identity for each hit in the BLAST analysis was then calculated by using the number of matches for a hit divided by the length of the primer, not the alignment length. Overall sequence identity was determined by calculating the maximum sequence identity of all hits for a specific primer against an individual organism and is reported in Table 5C and Table 5D. Primers with greater than 80% sequence identity were deemed as potentially cross-reactive. One primer deemed potentially cross reactive (sequence identity >0.8) was the F2 primer of orflab.II with B. pertussis; all other primers were not predicted to be cross-reactive (Table 5A and Table 5B). Since a single primer is predicted to be cross-reactive, we do not expect that our primer sets are cross-reactive with any of the organisms. We confirmed that these targets were not significantly cross-reactive experimentally using genomic extracts of these targets (Table 5E and Table 5F). One replicate of orf7ab.I was cross-reactive with HRSV Strain A2011 but was not deemed to be a concern since all three replicates did not amplify. The calculated sensitivity was 100% for both orflab.II and orf7ab.I and the calculated specificity was 100% and 99.13% for orflab.II and orf7ab.I, respectively.

The orf7ab.I and orflab.II primer sets were used to test cross-reactivity against several pathogens found in the upper respiratory tract of individuals presenting with symptoms similar to COVID-19. For each pathogen, 5 μL of genomic DNA/RNA at a concentration of 2×10³ copies/μL was used as a template to result in a total of 10⁴ copies/reaction. NTC reactions with water were used as negative controls, and heat-inactivated SARS-CoV-2 at a concentration of 2×10³ copies/μL to result in a total of 10⁴ copies/reaction was used as a positive control. Positive amplification was determined as any amplification at 30 minutes that was greater than 50% of the average fluorescent intensity value of the positive controls at 30 minutes. Sensitivity and specificity were calculated in the same manner as listed before. The pathogens used and their reactivity with orf7ab.I and orf7ab.II are displayed in Table 5E and Table 5F, respectively.

TABLE 5A
Primer Explorer V5 parameters used in the design of RT-LAMP primers. Default
values set by Primer Explorer upon selection of the parameter set are indicated by “−”.
Parameters not included in this table are kept at their default values.

| | | N.I | N.II | N.III | RdRP.I | RdRP.II | RdRP.III | Orf1ab.I | Orf1ab.II | Orf1ab.III | RegX |
|---|---|---|---|---|---|---|---|---|---|---|---|
| | Parameter Set | Normal | Normal | Normal | AT Rich | AT Rich | AT Rich | AT Rich | AT Rich | AT Rich | Normal |
| Distance | F2/B2 | − | 120-225 | 120-220 | − | − | − | − | − | − | − |
| between | F2/F3 | − | 0-30 | 0-40 | 0-30 | 0-25 | 0-25 | 0-25 | 0-25 | 0-25 | 0-35 |
| Primers | | | | | | | | | | | |
| Primer | F1c/B1c | − | − | 27-40 | − | − | − | − | − | − | − |
| Length (bp) | F2/B2 | − | − | 23-35 | − | − | − | − | − | − | − |
| | F3/B3 | − | − | 23-35 | − | − | − | − | − | − | − |
| | GC Content (%) | − | − | − | − | − | − | − | − | − | 30-65 |
| | ΔGₘᵢₙ (Dimerization) | − | − | -5.00 | − | − | − | − | − | − | -5.0 |
| | (kcal/mol) | | | | | | | | | | |
| | | Loop Primers | | | | | | | | | |
| | GC Content (%) | − | − | 10-80 | − | 10-65 | 10-65 | − | − | − | 10-90 |
| | ΔGₘᵢₙ (Dimerization) | − | − | -5.00 | − | − | − | − | − | − | -3.50 |
| | (kcal/mol) | | | | | | | | | | |
| | Melting Temp (° C.) | − | − | 50-66 | − | 50-66 | 50-66 | − | − | − | 50-66 |
| | Primer Length (bp) | − | − | 20-35 | − | − | − | − | − | − | − |

TABLE 5B
BLAST parameters used during in-silico homology analysis.
For the entrez query, “{TaxonID}” is
replaced with the TaxonID of the respective microorganism.

| Parameter | Value |
|---|---|
| Algorithm | blastn |
| Database | nt |
| Entrez Query | txid{TaxonID}[ORGN] |
| Expect threshold | 1000 |
| Alignments | 1000 |
| Match/Mistmatch Score | 1, −3 |
| Gap existence/extension | 5, 2 |

TABLE 5C
Results from the in-silico sequence identity analysis for the orf1ab.II primer set with
primers deemed to be potentially cross-reactive (sequence identity > 0.8).

| Taxon | TXID | F3 | B3 | LF | LB | F2 | F1c | B2 | B1c |
|---|---|---|---|---|---|---|---|---|---|
| Human coronavirus | 11137 | 0.32 | 0.37 | 0.29 | 0.37 | 0.37 | 0.35 | 0.37 | 0.32 |
| 229E | | | | | | | | | |
| Human coronavirus | 31631 | 0.36 | 0.42 | 0.33 | 0.42 | 0.42 | 0.40 | 0.42 | 0.36 |
| OC43 | | | | | | | | | |
| Human coronavirus | 290028 | 0.32 | 0.37 | 0.29 | 0.37 | 0.37 | 0.35 | 0.37 | 0.28 |
| HKU1 | | | | | | | | | |
| Human coronavirus | 277944 | 0.36 | 0.37 | 0.33 | 0.37 | 0.37 | 0.35 | 0.37 | 0.32 |
| NL63 | | | | | | | | | |
| SARS-coronavirus | 694009 | 0.32 | 0.37 | 0.29 | 0.37 | 0.37 | 0.35 | 0.37 | 0.28 |
| MERS-coronavirus | 1335626 | 0.41 | 0.47 | 0.38 | 0.47 | 0.47 | 0.45 | 0.47 | 0.36 |
| Human respirovirus 1 | 12730 | 0.32 | 0.37 | 0.33 | 0.37 | 0.37 | 0.35 | 0.37 | 0.32 |
| Human rubulavirus 2 | 1979160 | 0.32 | 0.37 | 0.29 | 0.37 | 0.37 | 0.35 | 0.37 | 0.28 |
| Human respirovirus 3 | 11216 | 0.41 | 0.42 | 0.50 | 0.42 | 0.42 | 0.40 | 0.42 | 0.36 |
| Human rubulavirus 4 | 1979161 | 0.32 | 0.37 | 0.29 | 0.37 | 0.37 | 0.35 | 0.37 | 0.28 |
| Influenza A Virus | 11320 | 0.55 | 0.53 | 0.46 | 0.53 | 0.53 | 0.50 | 0.53 | 0.48 |
| Influenza B Virus | 11520 | 0.41 | 0.58 | 0.42 | 0.47 | 0.47 | 0.45 | 0.47 | 0.40 |
| Human Enterovirus | 1193974 | 0.32 | 0.37 | 0.29 | 0.37 | 0.37 | 0.35 | 0.37 | 0.28 |
| Human Respiratory | 11250 | 0.45 | 0.47 | 0.42 | 0.47 | 0.47 | 0.45 | 0.47 | 0.40 |
| syncytial virus | | | | | | | | | |
| Rhinovirus A | 147711 | 0.36 | 0.37 | 0.33 | 0.37 | 0.37 | 0.40 | 0.37 | 0.32 |
| Rhinovirus B | 147712 | 0.32 | 0.37 | 0.29 | 0.37 | 0.37 | 0.35 | 0.37 | 0.28 |
| Rhinovirus C | 463676 | 0.36 | 0.37 | 0.33 | 0.37 | 0.37 | 0.40 | 0.37 | 0.32 |
| Chlamydia pneumoniae | 83558 | 0.41 | 0.47 | 0.38 | 0.47 | 0.47 | 0.45 | 0.47 | 0.36 |
| Haemophilus | 727 | 0.45 | 0.53 | 0.42 | 0.53 | 0.53 | 0.50 | 0.53 | 0.40 |
| influenzae | | | | | | | | | |
| Legionella pneumophila | 446 | 0.50 | 0.53 | 0.46 | 0.53 | 0.53 | 0.50 | 0.53 | 0.44 |
| Mycobacterium | 1773 | 0.00 | 0.58 | 0.00 | 0.58 | 0.58 | 0.55 | 0.63 | 0.48 |
| tuberculosis | | | | | | | | | |
| Streptococcus | 1313 | 0.50 | 0.53 | 0.46 | 0.53 | 0.53 | 0.55 | 0.53 | 0.44 |
| pneumoniae | | | | | | | | | |
| Streptococcus | 1314 | 0.50 | 0.58 | 0.46 | 0.58 | 0.58 | 0.55 | 0.58 | 0.44 |
| pyogenes | | | | | | | | | |
| Bordetella pertussis | 520 | 0.55 | 0.63 | 0.00 | 0.63 | 0.84 | 0.65 | 0.00 | 0.00 |
| Mycoplasma | 2104 | 0.45 | 0.47 | 0.42 | 0.47 | 0.47 | 0.45 | 0.47 | 0.40 |
| pneumoniae | | | | | | | | | |
| Pneumocystis jirovecii | 42068 | 0.32 | 0.37 | 0.29 | 0.37 | 0.37 | 0.35 | 0.37 | 0.28 |
| Candida albicans | 5476 | 0.45 | 0.53 | 0.42 | 0.53 | 0.53 | 0.50 | 0.53 | 0.40 |
| Pseudomonas | 287 | 0.55 | 0.63 | 0.50 | 0.63 | 0.63 | 0.60 | 0.63 | 0.48 |
| aeruginosa | | | | | | | | | |
| Staphylococcus | 1282 | 0.50 | 0.53 | 0.46 | 0.53 | 0.53 | 0.50 | 0.53 | 0.44 |
| epidermis | | | | | | | | | |
| Streptococcus salivarius | 1304 | 0.41 | 0.47 | 0.42 | 0.47 | 0.47 | 0.45 | 0.47 | 0.52 |

TABLE 5D
Results from the in-silico sequence identity analysis for the orf7ab.I primer set with
primers deemed to be potentially cross-reactive (sequence identity > 0.8).

| Taxon | TXID | F3 | B3 | LF | LB | F2 | F1c | B2 | Bic |
|---|---|---|---|---|---|---|---|---|---|
| Human coronavirus | 11137 | 0.39 | 0.30 | 0.32 | 0.35 | 0.39 | 0.32 | 0.30 | 0.29 |
| 229E | | | | | | | | | |
| Human coronavirus | 31631 | 0.44 | 0.35 | 0.36 | 0.40 | 0.44 | 0.36 | 0.35 | 0.33 |
| OC43 | | | | | | | | | |
| Human coronavirus | 290028 | 0.39 | 0.30 | 0.28 | 0.35 | 0.39 | 0.28 | 0.30 | 0.29 |
| HKU1 | | | | | | | | | |
| Human coronavirus | 277944 | 0.39 | 0.48 | 0.32 | 0.35 | 0.39 | 0.32 | 0.35 | 0.33 |
| NL63 | | | | | | | | | |
| SARS-coronavirus | 694009 | 0.39 | 0.30 | 0.28 | 0.35 | 0.39 | 0.28 | 0.30 | 0.29 |
| MERS-coronavirus | 1335626 | 0.50 | 0.39 | 0.36 | 0.45 | 0.50 | 0.36 | 0.39 | 0.38 |
| Human respirovirus 1 | 12730 | 0.39 | 0.35 | 0.32 | 0.35 | 0.39 | 0.32 | 0.35 | 0.33 |
| Human rubulavirus 2 | 1979160 | 0.39 | 0.30 | 0.28 | 0.35 | 0.39 | 0.28 | 0.30 | 0.29 |
| Human respirovirus 3 | 11216 | 0.44 | 0.35 | 0.36 | 0.40 | 0.44 | 0.36 | 0.35 | 0.38 |
| Human rubulavirus 4 | 1979161 | 0.39 | 0.30 | 0.28 | 0.35 | 0.39 | 0.28 | 0.30 | 0.29 |
| Influenza A Virus | 11320 | 0.56 | 0.57 | 0.44 | 0.55 | 0.56 | 0.44 | 0.48 | 0.46 |
| Influenza B Virus | 11520 | 0.50 | 0.48 | 0.48 | 0.65 | 0.50 | 0.40 | 0.43 | 0.42 |
| Human Enterovirus | 1193974 | 0.39 | 0.30 | 0.28 | 0.35 | 0.39 | 0.28 | 0.30 | 0.29 |
| Human Respiratory | 11250 | 0.50 | 0.48 | 0.48 | 0.45 | 0.50 | 0.40 | 0.48 | 0.54 |
| syncytial virus | | | | | | | | | |
| Rhinovirus A | 147711 | 0.39 | 0.35 | 0.32 | 0.45 | 0.39 | 0.32 | 0.43 | 0.33 |
| Rhinovirus B | 147712 | 0.39 | 0.30 | 0.28 | 0.35 | 0.39 | 0.28 | 0.30 | 0.29 |
| Rhinovirus C | 463676 | 0.39 | 0.35 | 0.32 | 0.40 | 0.39 | 0.32 | 0.35 | 0.33 |
| Chlamydia pneumoniae | 83558 | 0.50 | 0.39 | 0.36 | 0.45 | 0.50 | 0.36 | 0.39 | 0.38 |
| Haemophilus | 727 | 0.56 | 0.43 | 0.40 | 0.50 | 0.56 | 0.40 | 0.43 | 0.42 |
| influenzae | | | | | | | | | |
| Legionella pneumophila | 446 | 0.56 | 0.48 | 0.44 | 0.50 | 0.56 | 0.44 | 0.48 | 0.46 |
| Mycobacterium | 1773 | 0.61 | 0.00 | 0.48 | 0.55 | 0.61 | 0.60 | 0.52 | 0.50 |
| tuberculosis | | | | | | | | | |
| Streptococcus | 1313 | 0.56 | 0.48 | 0.44 | 0.55 | 0.56 | 0.44 | 0.48 | 0.46 |
| pneumoniae | | | | | | | | | |
| Streptococcus | 1314 | 0.56 | 0.48 | 0.44 | 0.55 | 0.56 | 0.44 | 0.48 | 0.46 |
| pyogenes | | | | | | | | | |
| Bordetella pertussis | 520 | 0.67 | 0.00 | 0.00 | 0.00 | 0.67 | 0.52 | 0.00 | 0.00 |
| Mycoplasma | 2104 | 0.50 | 0.43 | 0.40 | 0.45 | 0.50 | 0.40 | 0.43 | 0.42 |
| pneumoniae | | | | | | | | | |
| Pneumocystis jirovecii | 42068 | 0.39 | 0.30 | 0.28 | 0.35 | 0.39 | 0.28 | 0.30 | 0.29 |
| Candida albicans | 5476 | 0.50 | 0.43 | 0.40 | 0.50 | 0.50 | 0.40 | 0.43 | 0.67 |
| Pseudomonas | 287 | 0.61 | 0.52 | 0.48 | 0.60 | 0.61 | 0.48 | 0.52 | 0.50 |
| aeruginosa | | | | | | | | | |
| Staphylococcus | 1282 | 0.56 | 0.48 | 0.44 | 0.50 | 0.56 | 0.68 | 0.48 | 0.46 |
| epidermis | | | | | | | | | |
| Streptococcus salivarius | 1304 | 0.50 | 0.52 | 0.40 | 0.45 | 0.50 | 0.40 | 0.39 | 0.42 |

TABLE 5E
Pathogens used to test cross-reactivity with orf7ab.I and the
associated positive amplifications. Product numbers prefixed
by NR- were obtained through BEI Resources, NIAID, NIH; all others
were purchased from American Type Culture Collection (ATCC).

| | | Positive |
|---|---|---|
| Virus | Product Number | Amplifications |
| Influenza A (H1N1) | NR-2773 | 0/3 |
| Influenza A (H3N2) | NR-10045 | 0/3 |
| Influenza B | NR-45848 | 0/3 |
| MERS-CoV | NR-45843 | 0/3 |
| Staphylococcus epidermidis | NR-51362 | 0/3 |
| (VCU036) | | |
| SARS-CoV (Urbani) | NR-52346 | 0/3 |
| Betacoronavirus 1 (OC43) | VR-1558D | 0/3 |
| Enterovirus 71 (MP4) | NR-4961 | 0/3 |
| Enterovirus D68 | NR-49136 | 0/3 |
| Human Coronavirus (229E) | VR-740D | 0/3 |
| Human Coronavirus (NL63) | NR-44105 | 0/3 |
| Human Metapneumovirus | NR-49122 | 0/3 |
| (TN/83-1211) | | |
| HRSV (A2011/3-12) | NR-44227 | 1/3 |
| HRSV(B1) | NR-48831 | 0/3 |
| Human Adenovirus 11 | VR-12D | 0/3 |
| (Slobitski) | | |
| Human Adenovirus 3 (GB) | VR-847D | 0/3 |
| Human Adenovirus 4 (RI-67) | VR-1572D | 0/3 |
| Human Adenovirus 7 | VR-7D | 0/3 |
| (Gomen) | | |
| Candida albicans (12C) | NR-50307 | 0/3 |
| Mycobacterium Tuberculosis | NR-48669 | 0/3 |
| (H37Rv) | | |
| Human Rhinovirus 17 (33342) | VR-1663D | 0/3 |
| Human Parainfluenza Virus 1 | VR-94D | 0/3 |
| (C35) | | |
| Human Parainfluenza Virus 2 | VR-92D | 0/3 |
| (Greer) | | |
| Human Parainfluenza Virus 3 | VR-93D | 0/3 |
| (C243) | | |
| Haemophilus Influenzae | 51907D-5 | 0/3 |
| (KW20) | | |
| Legionella pneumophilia | 33152D-5 | 0/3 |
| (Philadelphia-1) | | |
| Streptococcus pyogenes (T1) | 12344D-5 | 0/3 |
| Streptococcus pneumoniae | 700669D-5 | 0/3 |
| (Klein) | | |
| Bordetella pertussis | BAA-1335D-5 | 0/3 |
| (MN2531) | | |
| Pseudomonas aeruginosa | 15442D-5 | 0/3 |
| Water (Negative) | — | 0/21 |
| HI SARS-CoV-2 (Positive) | VR-1986HK | 21/21 |
| Sensitivity | | 1.0 |
| Specificity | | 0.9913 |

TABLE 5F
Pathogens used to test cross-reactivity with orf1ab.II and the
associated positive amplifications. Product numbers prefixed by
NR- were obtained through BEI Resources, NIAID, NIH; all others
were purchased from American Type Culture Collection (ATCC).

| | | Positive |
|---|---|---|
| Virus | Product Number | Amplifications |
| Influenza A (H1N1) | NR-2773 | 0/3 |
| Influenza A (H3N2) | NR-10045 | 0/3 |
| Influenza B | NR-45848 | 0/3 |
| MERS-CoV | NR-45843 | 0/3 |
| Staphylococcus epidermidis | NR-51362 | 0/3 |
| (VCU036) | | |
| SARS-CoV (Urbani) | NR-52346 | 0/3 |
| Betacoronavirus 1 (OC43) | VR-1558D | 0/3 |
| Enterovirus 71 (MP4) | NR-4961 | 0/3 |
| Enterovirus D68 | NR-49136 | 0/3 |
| Human Coronavirus (229E) | VR-740D | 0/3 |
| Human Coronavirus (NL63) | NR-44105 | 0/3 |
| Human Metapneumovirus | NR-49122 | 0/3 |
| (TN/83-1211) | | |
| HRSV (A2011/3-12) | NR-44227 | 0/3 |
| HRSV(B1) | NR-48831 | 0/3 |
| Human Adenovirus 11 | VR-12D | 0/3 |
| (Slobitski) | | |
| Human Adenovirus 3 (GB) | VR-847D | 0/3 |
| Human Adenovirus 4 (RI-67) | VR-1572D | 0/3 |
| Human Adenovirus 7 | VR-7D | 0/3 |
| (Gomen) | | |
| Candida albicans (12C) | NR-50307 | 0/3 |
| Mycobacterium Tuberculosis | NR-48669 | 0/3 |
| (H37Rv) | | |
| Human Rhinovirus 17 (33342) | VR-1663D | 0/3 |
| Human Parainfluenza Virus 1 | VR-94D | 0/3 |
| (C35) | | |
| Human Parainfluenza Virus 2 | VR-92D | 0/3 |
| (Greer) | | |
| Human Parainfluenza Virus 3 | VR-93D | 0/3 |
| (C243) | | |
| Haemophilus Influenzae | 51907D-5 | 0/3 |
| (KW20) | | |
| Legionella pneumophilia | 33152D-5 | 0/3 |
| (Philadelphia-1) | | |
| Streptococcus pyogenes (T1) | 12344D-5 | 0/3 |
| Streptococcus pneumoniae | 700669D-5 | 0/3 |
| (Klein) | | |
| Bordetella pertussis | BAA-1335D-5 | 0/3 |
| (MN2531) | | |
| Pseudomonas aeruginosa | 15442D-5 | 0/3 |
| Water (Negative) | — | 0/15 |
| HI SARS-CoV-2 (Positive) | VR-1986HK | 15/15 |
| Sensitivity | | 1.0 |
| Specificity | | 1.0 |

### Example 5—Design and Screening of Primers

The following conserved genes of SARS-CoV-2 were targeted to design at least three primer sets per gene: the N gene, the RdRp gene, and the orflab segment using PrimerExplorer V5. Three experiments were performed using heat-inactivated SARS-CoV-2 to select the optimal primer set: (1) using a fluorescent RT-LAMP kit and pooled saliva to determine whether the primers could amplify the target in 18% saliva, which is the maximum concentration of saliva that can be achieved in a liquid format); (2) using a fluorescent RT-LAMP kit and water to determine whether the primers could dimerize (i.e., show amplification in non-template controls (NTC)); and (3) using a colorimetric RT-LAMP kit to determine the limit of detection (LoD) of the primer set.

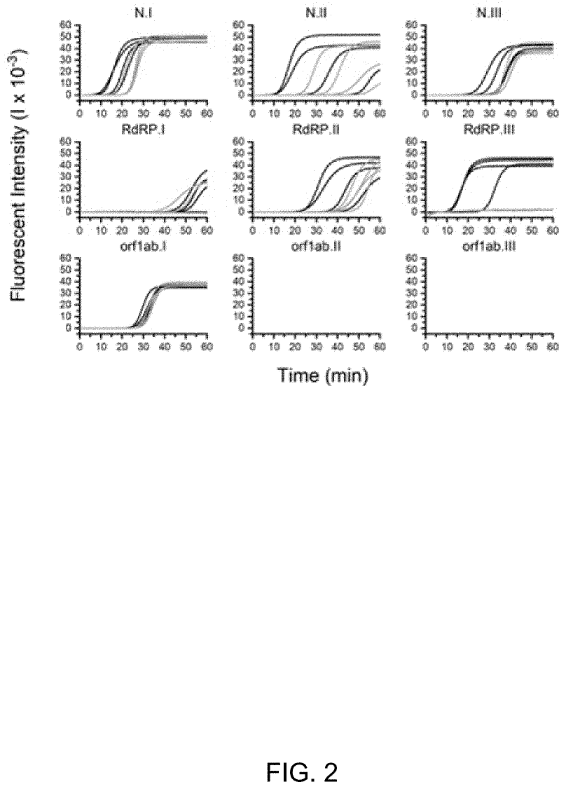

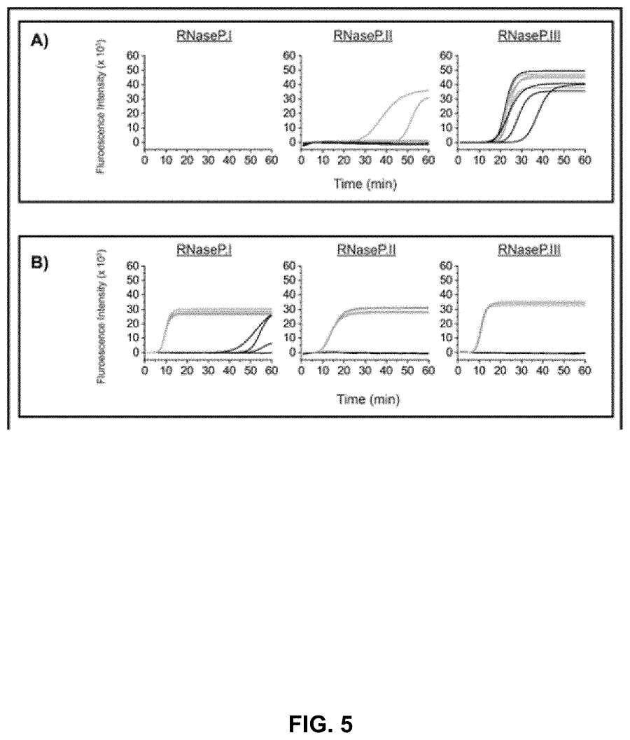

Primer sets were screened in water using a fluorescent RT-LAMP kit and in-vitro transcribed SARS-CoV-2 RNA for the gene targeted by the primer set to assess performance and ability to dimerize. Water was used to prevent any off-target interactions with the sample background. The assay utilized a no-primer control to ensure that the reaction zones do not change color when heated. Further screening to determine off-target interactions was conducted in 18% saliva using a fluorescent RT-LAMP kit and heat-inactivated SARS-CoV-2 to assess performance in complex samples. After screening the primer sets depicted in Table 6 and based on the results illustrated in FIGS. 2, 3, and 4, the orflab.II primer set, as depicted in Table 7, was the optimal primer set because it provided no false positives (in water and saliva) and had a LoD of 200 copies/4, of reaction (reaction volume 25 Similarly, a primer was designed to target RNaseP in saliva as a positive control to ensure that amplification could be obtained in saliva, as illustrated in FIG. 5.

TABLE 6

| Primer | Sequence (5′ - 3′) |
|---|---|
| N.I_F3 | TGGACCCCAAAATCAGCG |
| N.I_B3 | GCCTTGTCCTCGAGGGAAT |
| N.I_FIP | CCACTGCGTTCTCCATTCTGGTAAATGCACCCCGCATTACG |
| N.I_BIP | CGCGATCAAAACAACGTCGGCCCTTGCCATGTTGAGTGAGA |
| N.I_LF | GTTGAATCTGAGGGTCCACCA |
| N.I_LB | ACCCAATAATACTGCGTCTTGG |
| N.II_F3 | GCCCCAAGGTTTACCCAAT |
| N.II_B3 | AGCACCATAGGGAAGTCCAG |
| NII_FIP | CGCCTTGTCCTCGAGGGAATTCGTCTTGGTTCACCGCTC |
| NII_BIP | AGACGAATTCGTGGTGGTGACGTGGCCCAGTTCCTAGGTAG |
| N.II_LF | TCTTCCTTGCCATGTTGAGTG |
| N.II_LB | ATGAAAGATCTCAGTCCAAGATGG |
| N.III_F3 | AATTGGCTACTACCGAAGAGCTA |
| N.III_B3 | GTAGAAGCCTTTTGGCAATGTTG |
| N.III_FIP | GTCTTTGTTAGCACCATAGGGAAGTCCTGAAAGATCTCAGTCCAA |
| | GATGG |
| N.III_BIP | GGAGCCTTGAATACACCAAAAGATCACTTGAGGAAGTTGTAGCAC |
| | GATTG |
| N.III_LF | TGGCCCAGTTCCTAGGTAGTAGAAATA |
| N.III_LB | CGCAATCCTGCTAACAATGCTG |
| RdRP.I_F3 | CAGATGCATTCTGCATTGT |
| RdRP.I_B3 | ATTACCAGAAGCAGCGTG |
| RdRP.I_FIP | CAGTTGAAACTACAAATGGAACACCTACAGTGTTCCCACCTACA |
| RdRP.I_BIP | AGCTAGGTGTTGTACATAATCAGGAGGTCAGCAGCATACACAAG |
| RdRP.I_LF | TTTTCTCACTAGTGGTCCAAAACT |
| RdRP.I_LB | TGTAAACTTACATAGCTCTAGACTT |
| RdRP.II_F3 | ACTATGACCAATAGACAGTTTCA |
| RdRP.II_B3 | GGCCATAATTCTAAGCATGTT |
| RdRP.II_FIP | GCCAACCACCATAGAATTTGCTAATAGCCGCCACTAGAGG |
| RdRP.II_BIP | AGTGATGTAGAAAACCCTCACCTAGGCATGGCTCTATCACAT |
| RdRP.II_LF | GTTCCAATTACTACAGTAGC |
| RdRP.II_LB | ATGGGTTGGGATTATCCTAA |
| RdRP.III_F3 | CGATAAGTATGTCCGCAATT |
| RdRP.III_B3 | ACTGACTTAAAGTTCTTTATGCT |
| RdRP.III_FIP | ATGCGTAAAACTCATTCACAAAGTCCAACACAGACTTTATGAGTG |
| | TC |
| RdRP.III_BIP | TGATACTCTCTGACGATGCTGTAGCCACTAGACCTTGAGAT |
| RdRP.III_LF | TGTGTCAACATCTCTATTTCTATAG |
| RdRP.III_LB | TGTGTGTTTCAATAGCACTTATGC |
| orf1ab.I_F3 | AGCTGGTAATGCAACAGAA |
| orf1ab.I_B3 | CACCACCAAAGGATTCTTG |
| orf1ab.I_FIP | TCCCCCACTAGCTAGATAATCTTTGCCAATTCAACTGTATTATCTTT |
| | CTG |
| orf1ab.I_BIP | GTGTTAAGATGTTGTGTACACACACATCCATATTGGCTTCCGG |
| orf1ab.I_LF | GCTTTAGCAGCATCTACAGCA |
| orf1ab.I_LB | TGGTACTGGTCAGGCAATAACAGT |
| orf1ab.II_F3 | ACTTAAAAACACAGTCTGTACC |
| orf1ab.II_B3 | TCAAAAGCCCTGTATACGA |
| orf1ab.II_FIP | TGACTGAAGCATGGGTTCGCGTCTGCGGTATGTGGAAAG |
| orf1ab.II_BIP | GCTGATGCACAATCGTTTTTAAACGCATCAGTACTAGTGCCTGT |
| orf1ab.II_LF | GAGTTGATCACAACTACAGCCATA |
| orf1ab.II_LB | TTGCGGTGTAAGTGCAGCC |
| orf1ab.III_F3 | TTGTGCTAATGACCCTGT |
| orf1ab.III_B3 | TCAAAAGCCCTGTATACGA |
| orf1ab.III_FIP | GATCACAACTACAGCCATAACCTTTGGGTTTTACACTTAAAAACAC |
| | AG |
| orf1ab.III_BIP | TGATGCACAATCGTTTTTAAACGGCATCAGTACTAGTGCCTGT |
| orf1ab.III_LF | CCACATACCGCAGACGGTACAG |
| orf1ab.III_LB | GGTGTAAGTGCAGCCCGT |
| RNaseP.I_F3 | TCAGGGTCACACCCAAGT |
| RNaseP.I_B3 | CGCATACACACACTCAGGAA |
| RNaseP.I_FIP | ACATGGCTCTGGTCCGAGGTCCTCCACTTATCCCCTCCGTG |
| RNaseP.I_BIP | CTGTTCCCTGGAAGCCCAAAGGTAACTGGGCCCACCAAGAG |
| RNaseP.I_LF | ACTCAGCATGCGAAGAGCCATAT |
| RNaseP.I_LB | CTGCATTGAGGGTGGGGGTAAT |
| RNaseP.II_F3 | GCCCTGTGGAACGAAGAG |
| RNaseP.II_B3 | TCCGTCCAGCAGCTTCTG |
| RNaseP.II_FIP | CACTGGATCCAGTTCAGCCTCCGCACACAGCATGGCAGAA |
| RNaseP.II_BIP | TTAGGAAAAGGCTTCCCAGCCGTGGGCCTTAAAGTCCGTCTT |
| RNaseP.II_LF | CACCGCGGGGCTCTCGGT |
| RNaseP.II_LB | CTGCCCCGGAGACCCAATG |
| RNaseP.III_F3 | TACATTCACGGCTTGGGC |
| RNaseP.III_B3 | GGGTGTGACCCTGAAGACT |
| RNaseP.III_FIP | CACCTGCAAGGACCCGAAGCAACCGCGCCATCAACATC |
| RNaseP.III_BIP | GCCAATACCTCCACCGTGGAGGTTGCGGATCCGAGTCAG |
| RNaseP.III_LF | CGCCTGCAGCTGCAGCGC |
| RNaseP.III_LB | GTTGATGAGCTGGAGCCAGAGA |

TABLE 7

| Primer | Sequence (5′ - 3′) |
|---|---|
| orf1ab.II_F3 | ACTTAAAAACACAGTCTGTACC |
| orf1ab.II_B3 | TCAAAAGCCCTGTATACGA |
| orf1ab.II_FIP | TGACTGAAGCATGGGTTCGCGTCTGCGGTATGTGG |
| | AAAG |
| orf1ab.II_BIP | GCTGATGCACAATCGTTTTTAAACGCATCAGTACT |
| | AGTGCCTGT |
| orf1ab.II_LF | GAGTTGATCACAACTACAGCCATA |
| orf1ab.II_LB | TTGCGGTGTAAGTGCAGCC |
| RNaseP.III_F3 | TACATTCACGGCTTGGGC |
| RNaseP.III_B3 | GGGTGTGACCCTGAAGACT |
| RNaseP.III_ | CACCTGCAAGGACCCGAAGCAACCGCGCCATCAAC |
| FIP | ATC |
| RNaseP.III_ | GCCAATACCTCCACCGTGGAGGTTGCGGATCCGAG |
| BIP | TCAG |
| RNaseP.III_LF | CGCCTGCAGCTGCAGCGC |
| RNaseP.III_LB | GTTGATGAGCTGGAGCCAGAGA |

As illustrated in FIG. 2, RT-qLAMP amplification curves for varying primer sets in saliva at a final concentration of 18% were generated. Blue lines indicate a positive control, wherein 5 μL of heat-inactivated SARS-CoV-2 was spiked into saliva and was added to the reaction mix to result in a final concentration of 1.0×10⁵ viral genome copies per reaction. Black lines indicate a non-template control (NTC), wherein 5 μL of saliva diluted 9:10 with water was added to the reaction mix.

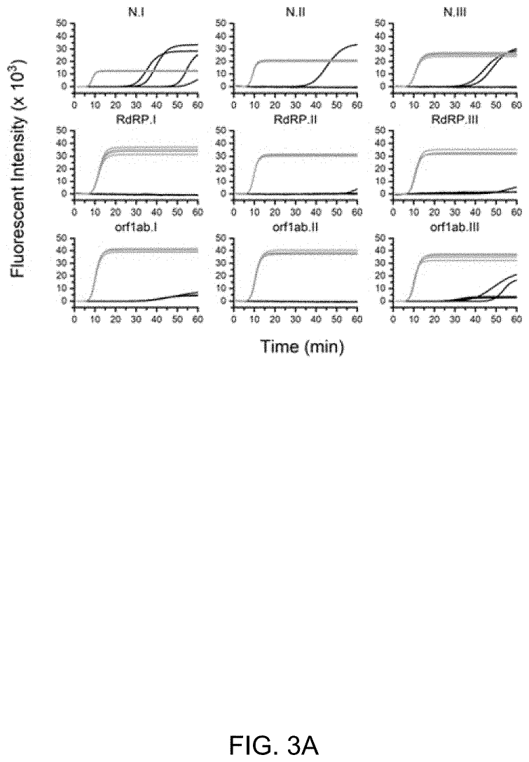

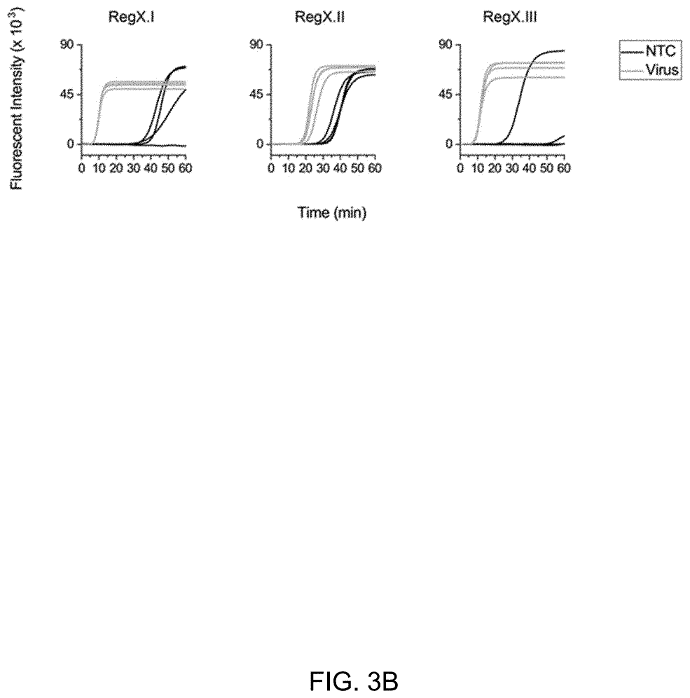

As illustrated in FIG. 3A, RT-qLAMP amplification curves for varying primer sets in water were generated. Blue lines indicate positive control, wherein 5 μL of 0.2 ng/μL: A) N gene synthetic RNA template, B) RNA-dependent RNA Polymerase (RdRP) synthetic RNA template, or C) orflab synthetic RNA template was added to the reaction. Black lines indicate non-template controls (NTC), wherein 5 μL of water was added instead of the template synthetic RNA. Four replicates of each condition were run per primer set.

As illustrated in FIG. 3B, RT-qLAMP fluorometric results of Region X primer sets in 18% saliva. Blue lines indicate positive controls where 5 μL of heat-inactivated SARS-CoV-2 added to the reaction mix to result in a final concentration of 1.0×10⁵ viral genome copies per reaction. Black lines indicate non-template control (NTC) where 5 μL of human saliva diluted to 90% with nuclease-free water was added to the reaction mix. Reactions had a final volume of 25 and used NEB 2×Fluorometric master mix. Reactions were run on a qTower3G with a ramp rate of 0.1° C./s

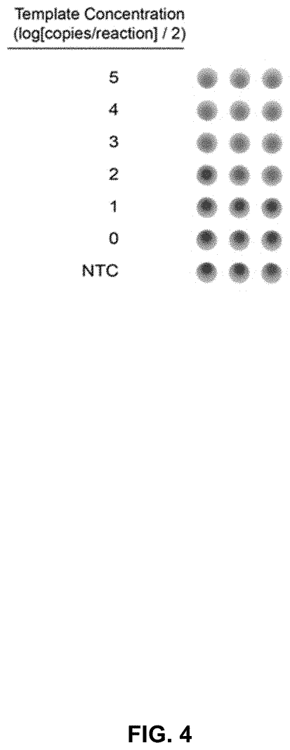

As illustrated in FIG. 4, colorimetric RT-LAMP scan images for limit of detection (LoD) of varying orflab and RdRP primer sets were generated. Yellow wells indicate a successful LAMP reaction taking place, whereas red/orange wells indicate absent or low-level amplifications respectively. 20 μL reaction mixtures were spiked with 5 μL of heat-inactivated virus dilutions in water at the labeled concentrations. Endpoint images were taken after incubating the plate at 65° C. for 60 minutes. Three replicates for each viral concentration were run per primer set.

As illustrated in FIG. 5, fluorometric RT-qLAMP results for primer sets targeting human RNaseP POP7 gene were generated in: A) 18% saliva spiked with 10⁵ genome equivalents/reaction of heat-inactivated SARS-CoV-2; B) water with 0.2 ng of synthetic RNaseP POP7 RNA; and C) colorimetric RT-LAMP LoD in 18% saliva spiked with 10⁵ genome equivalents/reaction of heat-inactivated SARS-CoV-2.

### Example 6—Primer Design

RT-LAMP primer sets were designed using PrimerExplorer v5. Parameters used to design primers can be found in Table 8. All other Primer Explorer parameters that are not indicated in Table 8 were set to the default values.

TABLE 8

| | | N.I | N.II | N.III | RdRP.I | RdRP.II | RdRP.III |
|---|---|---|---|---|---|---|---|
| | Parameter Set | Normal | Normal | Normal | AT Rich | AT Rich | AT Rich |
| Distance | F2/B2 | − | 120-225 | 120-220 | − | − | − |
| | F2/1F3 | − | 0-30 | 0-40 | 0-30 | 0-25 | 0-25 |
| Primer | F1c/B1c | − | − | 27-40 | − | − | − |
| | F2/B2 | − | − | 23-35 | − | − | − |
| | F3/B3 | − | − | 23-35 | − | − | − |
| | GC Content | − | − | − | − | − | − |
| | (%) | | | | | | |
| | ΔGₘᵢₙ (Dimerization) | − | − | −5.00 | − | − | − |
| | (kcal/mol) | | | | | | |
| | | Loop Primers | | | | | |
| | GC Content | − | − | 10-80 | − | 10-65 | 10-65 |
| | (%) | | | | | | |
| | ΔGₘᵢₙ (Dimerization) | − | − | −5.00 | − | − | − |
| | (kcal/mol) | | | | | | |
| | Melting | − | − | 50-66 | − | 50-66 | 50-66 |
| | Temp (° C.) | | | | | | |
| | Primer | − | − | 20-35 | − | − | − |
| | Length (bp) | | | | | | |
| | | Orf1ab.I | Orf1ab.II | Orf1ab.III | RNaseP.I | RNaseP.II | RNaseP.III |
| | Parameter Set | AT Rich | AT Rich | AT Rich | Normal | Normal | Normal |
| Distance | F2/B2 | − | − | − | − | − | − |
| | F2/1F3 | 0-25 | 0-25 | 0-25 | − | − | − |
| Primer | F1c/B1c | − | − | − | − | − | − |
| | F2/B2 | − | − | − | − | − | − |
| | F3/B3 | − | − | − | − | − | − |
| | GC Content | − | − | − | − | − | − |
| | (%) | | | | | | |
| | ΔGₘᵢₙ (Dimerization) | − | − | − | − | − | − |
| | (kcal/mol) | | | | | | |
| | | Loop Primers | | | | | |
| | GC Content | − | − | − | 40-99 | 40-99 | 40-99 |
| | (%) | | | | | | |
| | ΔGₘᵢₙ (Dimerization) | − | − | − | − | − | − |
| | (kcal/mol) | | | | | | |
| | Melting | − | − | − | 60-80 | 60-80 | 60-80 |
| | Temp (° C.) | | | | | | |
| | Primer | − | − | − | − | − | − |
| | Length (bp) | | | | | | |

Primer sets were designed using portions of the SARS-CoV-2 reference genome (NCBI accession number: NC 045512). Primer sets for RdRP were designed by first splitting the nsp12 gene sequence into 2 portions. Primer set RdRP.I was designed using the first portion of the nsp12 sequence, while primer sets RdRP.II and RdRP.III were designed using the second portion of the nsp12 sequence. Primer sets for orflab were designed using a portion of the orflab gene sequence. Primer sets for RNaseP were designed using the mRNA sequence for the POP7 gene, which encodes for the p20 subunit of RNaseP.

### Example 7—Effect of Mixed Primers

In order to increase the speed of the RT-LAMP reaction, the inclusion of multiple primer sets in the fluorescent RT-LAMP reaction mix was investigated. The investigation was carried out in water using NEB LAMP fluorescent dye as a fluorometric indicator. The inclusion of multiple primer sets did not seem to increase the reaction speed significantly. Rather, the reaction proceeded primarily at the speed of the primer set that had the fastest reaction time when used in isolation.

### Example 8—Primer Limit of Detection

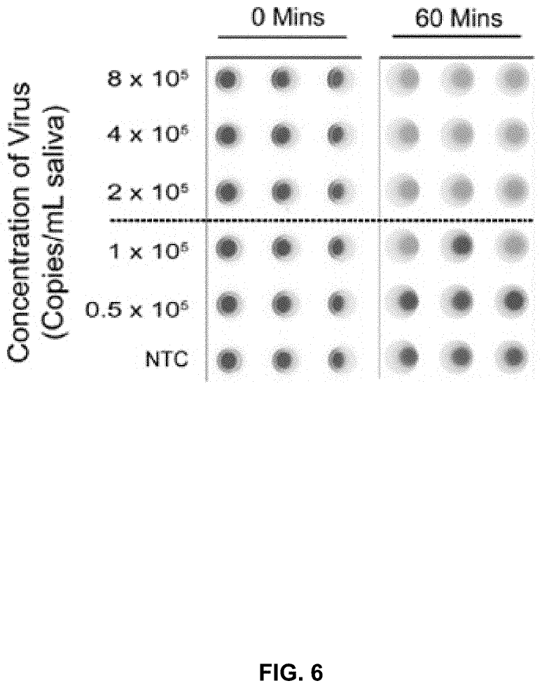

As illustrated in FIG. 6, the limit of detection in fresh saliva was determined for the orf7ab primer set. Fresh saliva was collected using a drooling method. The saliva was diluted 1:4 in water to obtain 20% saliva. Heat-inactivated SARS-CoV-2 was spiked into the 20% saliva with serial dilutions. A non-template control (NTC) was used as 20% saliva without the spiked virus. 5 μl of 20% saliva was added to 20 μl RT-LAMP reagents to obtain a total concentration of saliva of 5%. After incubation at 65° C. for 60 minutes, the color changed as illustrated in FIG. 6. In the figure, the number of copies on the y-axis represents the original concentration of the 100% saliva (i.e., before dilution). The limit of detection for orf7ab was 250 copies per reaction in a volume of 25 μl, which is equivalent to 2×10⁵ copies/mL of saliva. That is, the color change from red to yellow (which indicates a positive result) can be consistently achieved for 2×10⁵ copies/mL of saliva when the primer set is orf7ab.

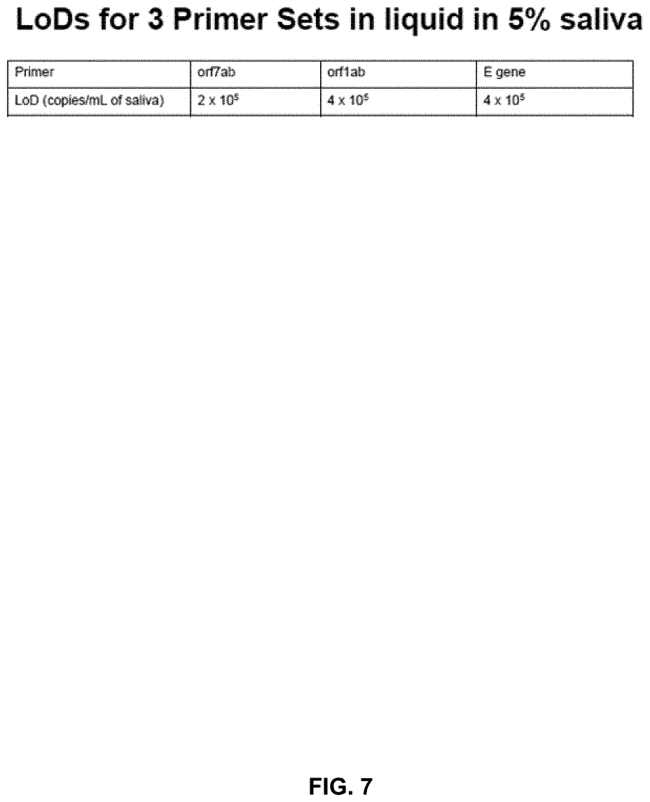

As illustrated in FIG. 7, the limit of detection for the orf7ab primer set was 2×10⁵ copies/mL of saliva; the limit of detection for the orflab primer set was 4×10⁵ copies/mL of saliva; and the limit of detection for the E gene primer set was 4×10⁵ copies/mL of saliva.

### Example 9—Sample LAMP Protocol

A sample list of materials used in a LAMP protocol can be found in Table 9.

TABLE 9

| | | Cost per |
|---|---|---|
| | Provider (Catalog | reaction |
| Material | Number) | ($) |
| Orf1ab.II Primer Set | Life Technologies (N/A) | 0.02 |
| RNaseP.III Primer Set | Life Technologies (N/A) | 0.09 |
| SARS-CoV-2 Rapid | New England Biolabs | 1.33 |
| Colorimetric LAMP | (E2019S) | |
| Assay Kit | | |
| Total | — | 1.44 |

Primer Mix

The primer mix was formulated by: (1) Obtaining all 6 diluted primers (100 μM) from the freezer, (2) Mixing 80 μl of FIP, 80 μl of BIP, 20 μl of FB, 20 μl LB, 10 μl of F3 and 10 μl of B3 in a tube; and (3) Adding enough PCR-grade water to reach 500 μl total.

### LAMP

1. Obtain the NEB Bst 2.0 Warmstart kit and the primer mix; 2. While the reagents thaw and after at least 5 minutes of spraying the RNaseAway, wipe the surfaces with a Kimwipe; 3. Label all the PCR tubes needed with the DNA sample and primers that will be used. Make sure to add a negative control which will not have DNA added; 4. Add 5 μl of PCR-grade water (or dye), 12.5 μl of NEB Bst 2.0 Warmstart kit and 2.5 μl of primer mix per reaction. A master mix can be made for however many reactions will be run; 5. If 5 μl of EBT dye are added, it should be in 1500 μM concentration so that the final concentration ends up being 30004; 6. The reactions with no DNA should have an extra 5 μl of PCR-grade water added and not opened again until they have to be loaded on a gel; 7. Once ready, the PCR tubes should be put in the PCR tray previously left in the pass-through chamber and carried out to a different room; 8. Once in the new room, obtain the sample DNA from the −20° C. freezer; 9. Spray your hands with RNaseAway spray and rub your hands around the DNA sample tube so that it is covered in the spray as well; 10. Add 5 μl of the DNA sample where appropriate and close the tubes. Avoid opening 2 DNA tubes at the same time and close the PCR tubes right after adding the DNA; 11. Put the samples in a thermocycler set at 65° C. for 1 hour and 80° C. for 5 minutes (samples may be kept at −20° C. overnight after this operation).

### Example 10—Comparative Primer Set Performance—Regions X1.1 and X1.2

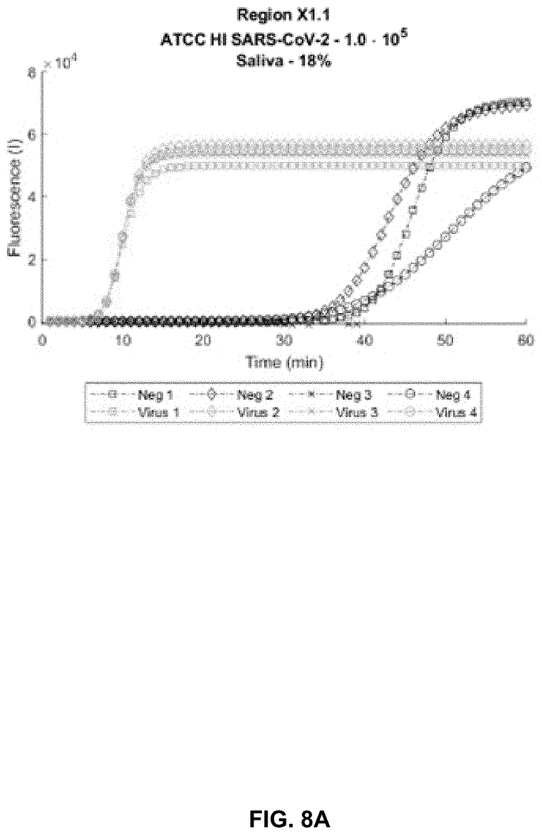

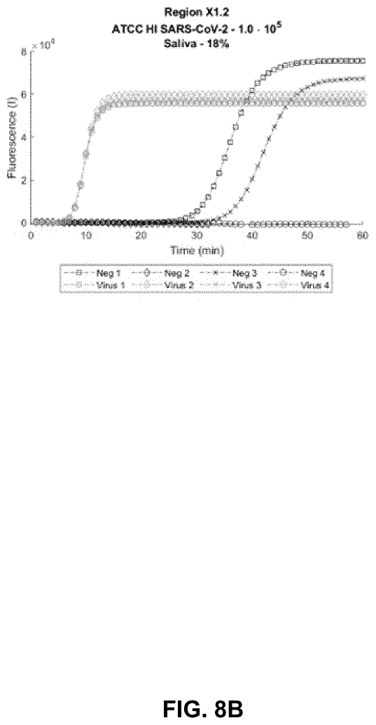

As illustrated in FIG. 8A, a graph of intensity of fluorescence over time in minutes was generated using 4 samples with a spiked SARS-CoV-2 virus in 18% saliva and 4 samples without the spiked SARS-CoV-2 virus in 18% saliva for the Region X1.1. Black lines indicate a non-template control (NTC), wherein 5 μL of saliva diluted to 18% in water was added to the reaction mix. Green lines indicate the samples spiked with SARS-CoV-2 in an amount of 100 k copies.

As shown in the figure, the virus-spiked samples reach a fluorescence of about 5×10⁴ in intensity between 7 to 13 minutes after commencement of the reaction, while the control samples reach a fluorescence of about 5×10⁴ in intensity between 45-60 minutes after commencement of the reaction.

As illustrated in FIG. 8B, a graph of intensity of fluorescence over time in minutes was generated using 4 samples with a spiked SARS-CoV-2 virus in 18% saliva and 4 samples without the spiked SARS-CoV-2 virus in 18% saliva for the Region X1.2. Black lines indicate a non-template control (NTC), wherein 5 μL of saliva diluted to 18% in water was added to the reaction mix. Green lines indicate the samples spiked with SARS-CoV-2.

As shown in the figure, the virus-spiked samples reach a fluorescence of about 5×10⁴ in intensity between 10 to 12 minutes after commencement of the reaction, while the control samples reach a fluorescence of about 5×10⁴ in intensity between 35-45 minutes after commencement of the reaction

Based on the data presented in FIGS. 8A-8B, the Region X1.1 and X1.2 primer sets did not provide reliable results for detecting SARS-CoV-2 at varying concentrations of SARS-CoV-2 in comparison to the Reg X3.1 primer set.

### Example 11—Comparative Primer Set Performance—Region X1.1

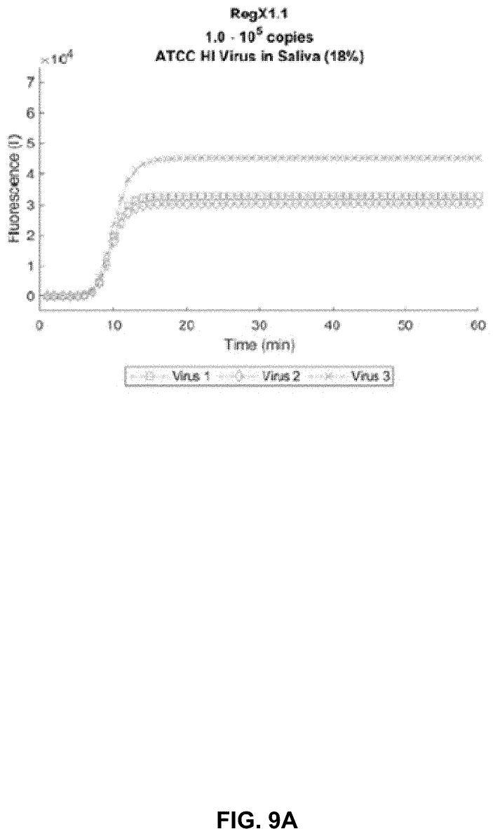

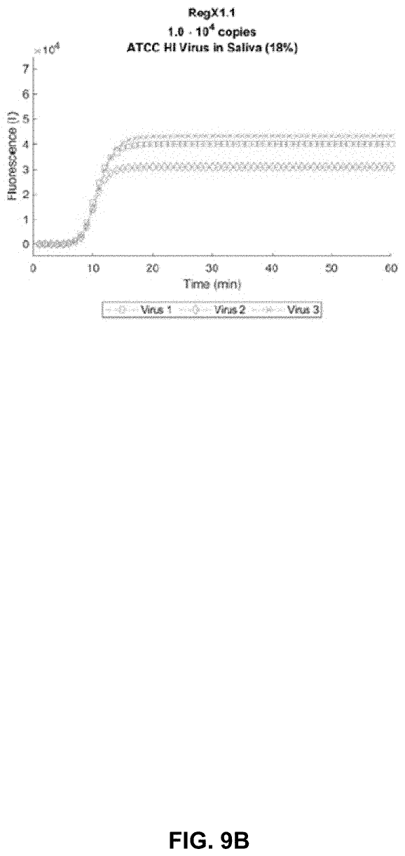

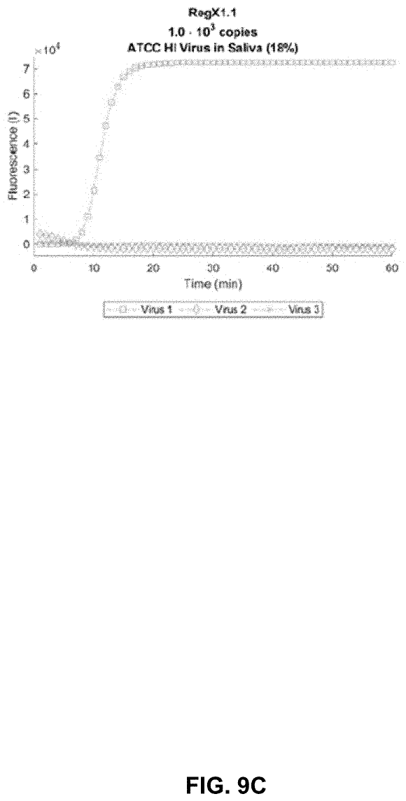

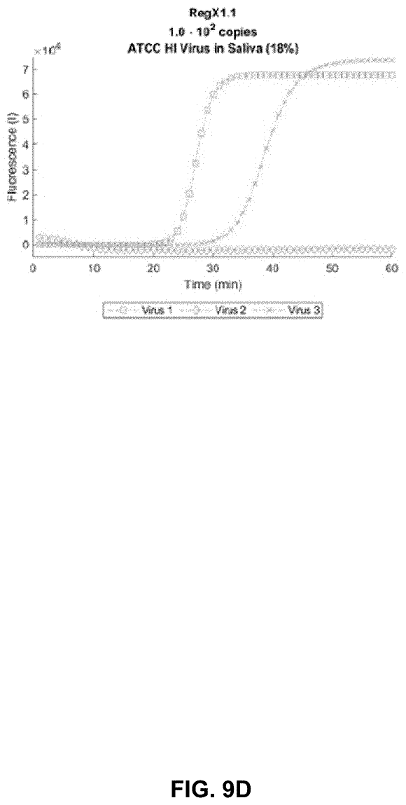

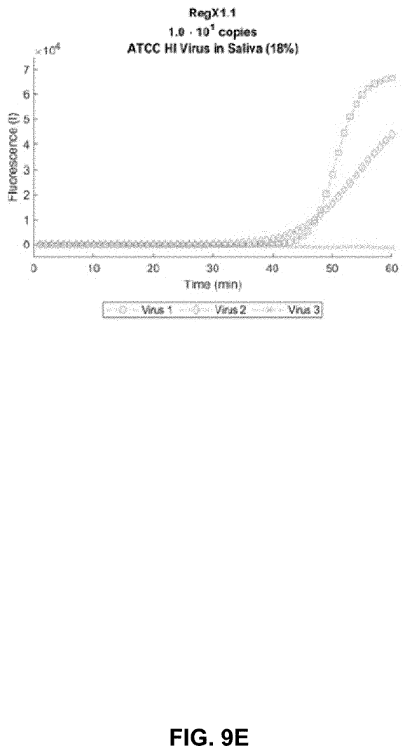

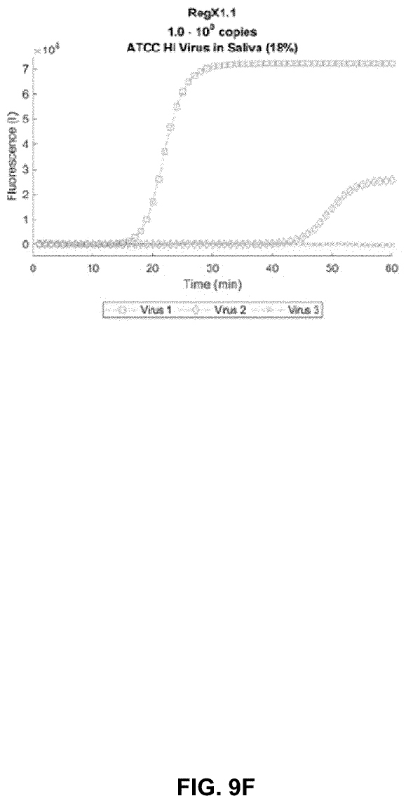

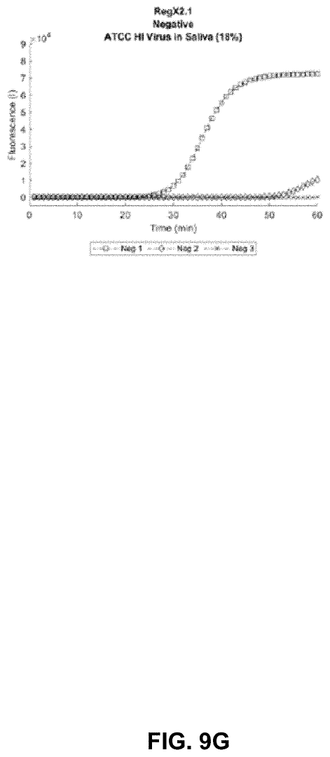

As illustrated in FIGS. 9A-9G, a graph of intensity of fluorescence over time in minutes was generated using 3 samples with a spiked SARS-CoV-2 virus in 18% saliva and 3 samples without the spiked SARS-CoV-2 virus in 18% saliva for the Region X1.1. Black lines indicate a non-template control (NTC), wherein 5 μL of saliva diluted to 18% in water was added to the reaction mix. Green lines indicate the samples spiked with SARS-CoV-2.

As shown in FIG. 9A, the three virus-spiked samples reach a fluorescence of about 3×10⁴ in intensity 10 minutes after commencement of the reaction for a viral concentration of about 100 k copies of the SARS-CoV-2 virus.

As illustrated in FIG. 9B, the three virus-spiked samples reached a fluorescence of about 3×10⁴ in intensity 10 minutes after commencement of the reaction for a viral concentration of about 10 k copies of the SARS-CoV-2 virus.

As illustrated in FIG. 9C, one of the three virus-spiked samples reached a fluorescence of about 3×10⁴ in intensity 10 minutes after commencement of the reaction for a viral concentration of about 1 k copies of the SARS-CoV-2 virus. The other two of the three virus-spiked samples did not exhibit a spike in fluorescence above the baseline level.

As illustrated in FIG. 9D, one of the three virus-spiked samples reached a fluorescence of about 7×10⁴ in intensity 20 minutes after commencement of the reaction for a viral concentration of about 100 copies of the SARS-CoV-2 virus. Another one of the three virus-spiked samples reached a fluorescence of about 7×10⁴ in intensity 40 minutes after commencement of the reaction for a viral concentration of about 100 copies of the SARS-CoV-2 virus. The other one of the three virus-spiked samples did not exhibit a spike in fluorescence above the baseline level.

As illustrated in FIG. 9E, one of the three virus-spiked samples reached a fluorescence of about 4×10⁴ in intensity 50 minutes after commencement of the reaction for a viral concentration of about 10 copies of the SARS-CoV-2 virus. Another one of the three virus-spiked samples reached a fluorescence of about 4×10⁴ in intensity 55 minutes after commencement of the reaction for a viral concentration of about 10 copies of the SARS-CoV-2 virus. The other one of the three virus-spiked samples did not exhibit a spike in fluorescence above the baseline level.

As illustrated in FIG. 9F, one of the three virus-spiked samples reached a fluorescence of about 7×10⁴ in intensity 25 minutes after commencement of the reaction for a viral concentration of about 1 copy of the SARS-CoV-2 virus. Another one of the three virus-spiked samples reached a fluorescence of about 3×10⁴ in intensity 55 minutes after commencement of the reaction for a viral concentration of about 1 copy of the SARS-CoV-2 virus. The other one of the three virus-spiked samples did not exhibit a spike in fluorescence above the baseline level.

As illustrated in FIG. 9G, for the controls that were not spiked with SARS-CoV-2 virus, one of the three samples reached a fluorescence of about 4×10⁴ in intensity 50 minutes after commencement of the reaction for a viral concentration of about 1 copy of the SARS-CoV-2 virus. The other two of the three virus-spiked samples did not exhibit a spike in fluorescence above the baseline level.

Based on the data presented in FIGS. 9A-9G, the Region X1.1 primer set did not provide reliable results for detecting SARS-CoV-2 at varying concentrations of SARS-CoV-2 in comparison to the Reg X3.1 primer set.

### Example 12—Comparative Primer Set Performance—Regions X2.1-X2.4

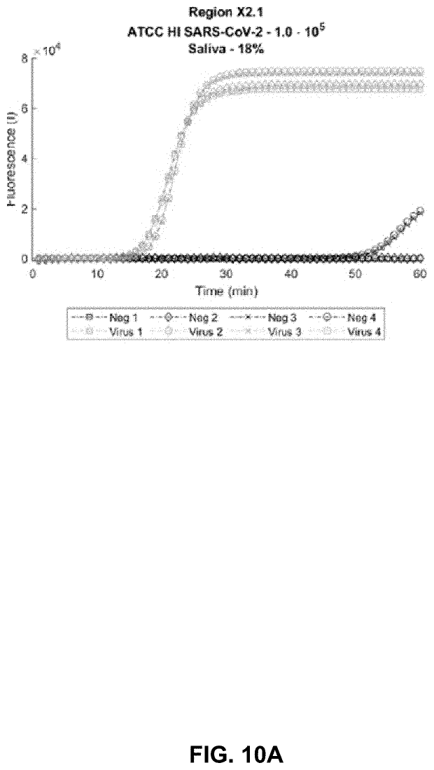

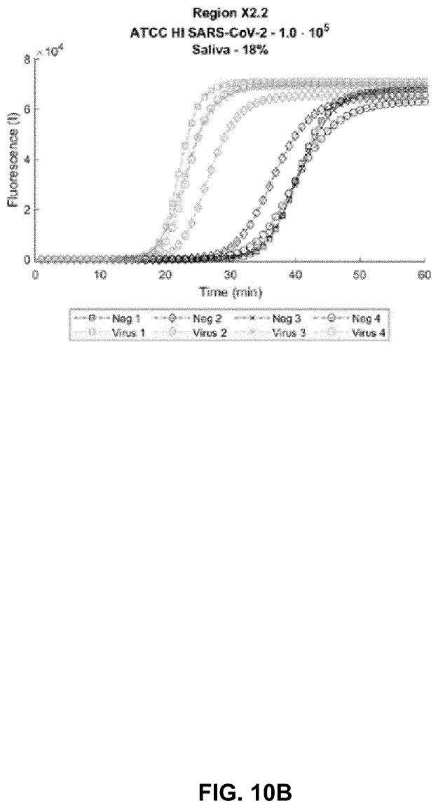

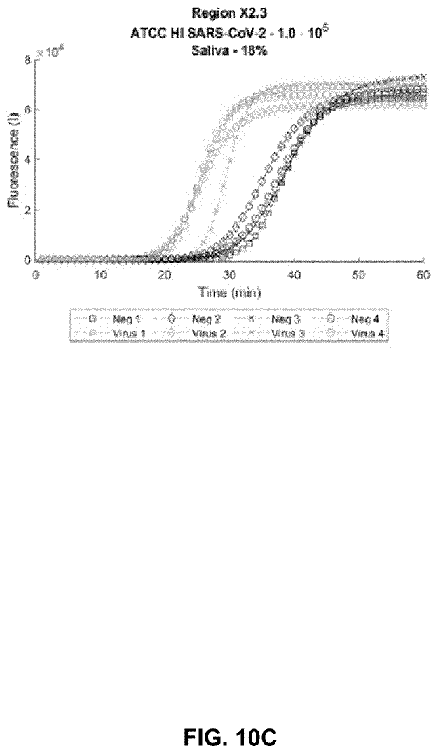

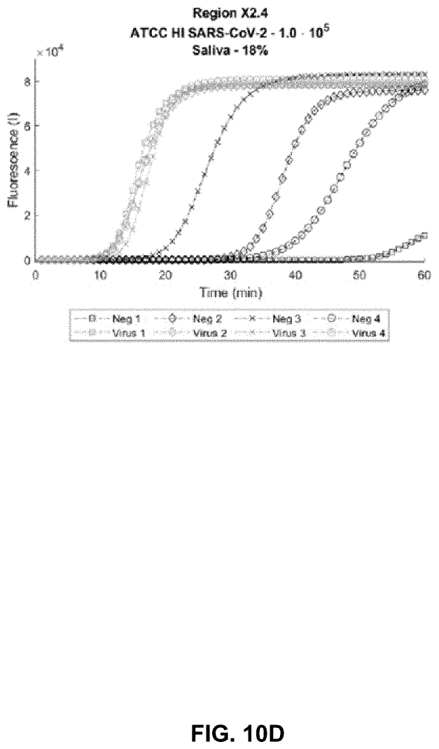

As illustrated in FIG. 10A-10D, a graph of intensity of fluorescence over time in minutes was generated using 4 samples with a spiked SARS-CoV-2 virus in 18% saliva and 4 samples without the spiked SARS-CoV-2 virus in 18% saliva for the Regions X2.1-X2.4. Black lines indicate a non-template control (NTC), wherein 5 μL of saliva diluted to 18% in water was added to the reaction mix. Green lines indicate the samples spiked with SARS-CoV-2 at an amount of 100 k copies.

As illustrated in FIG. 10A, the four virus-spiked samples reached a fluorescence of about 6×10⁴ in intensity 20 minutes after commencement of the reaction for a viral concentration of about 10 k copies of the SARS-CoV-2 virus when using primer sets drawn from Region X2.1. The controls did not spike until after 50 minutes.

As illustrated in FIG. 10B, the four virus-spiked samples reached a fluorescence of about 6×10⁴ in intensity 20-30 minutes after commencement of the reaction for a viral concentration of about 10 k copies of the SARS-CoV-2 virus when using primer sets drawn from Region X2.2. The controls did not spike until after 40 minutes.

As illustrated in FIG. 10C, the four virus-spiked samples reached a fluorescence of about 6×10⁴ in intensity 20-30 minutes after commencement of the reaction for a viral concentration of about 10 k copies of the SARS-CoV-2 virus when using primer sets drawn from Region X2.3. The controls did not spike until after 40 minutes.

As illustrated in FIG. 10D, the four virus-spiked samples reached a fluorescence of about 6×10⁴ in intensity 10-20 minutes after commencement of the reaction for a viral concentration of about 10 k copies of the SARS-CoV-2 virus when using primer sets drawn from Region X2.4. The controls did not spike until after 30 minutes.

Based on the data presented in FIGS. 10A-10D, the Region X2.1-X2.4 primer sets did not provide reliable results for detecting SARS-CoV-2 in comparison to the Reg X3.1 primer set.

### Example 13—Comparative Primer Set Performance—Region X2.1

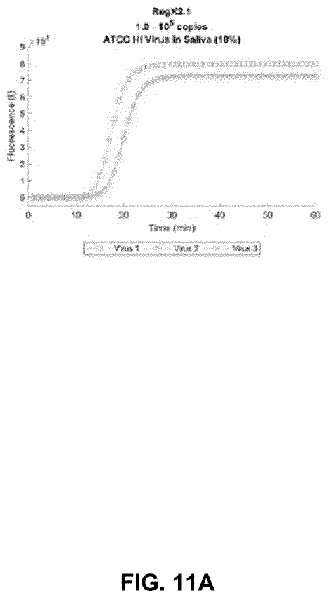

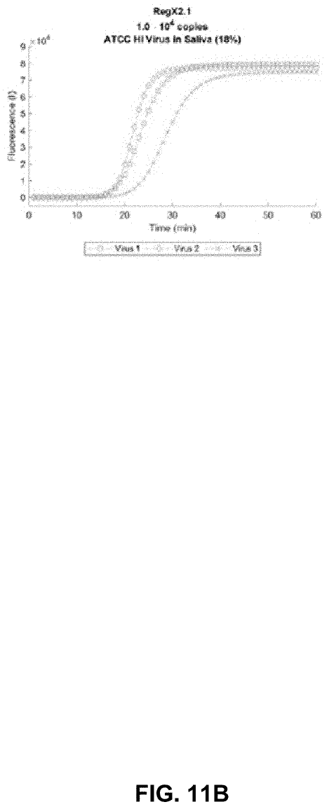

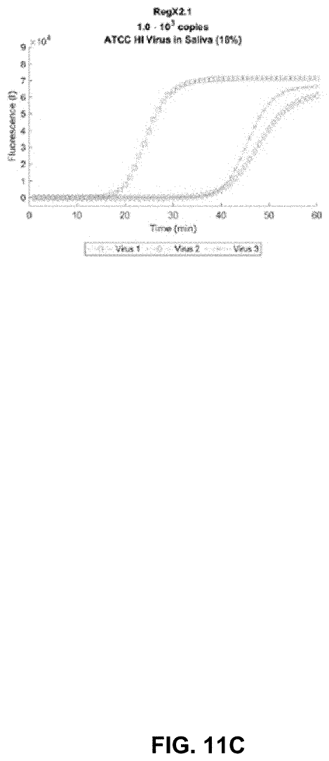

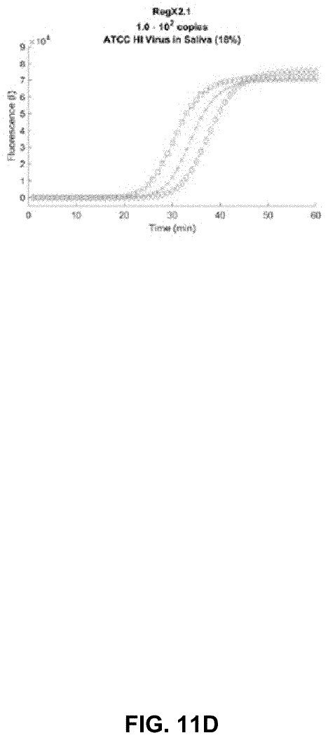

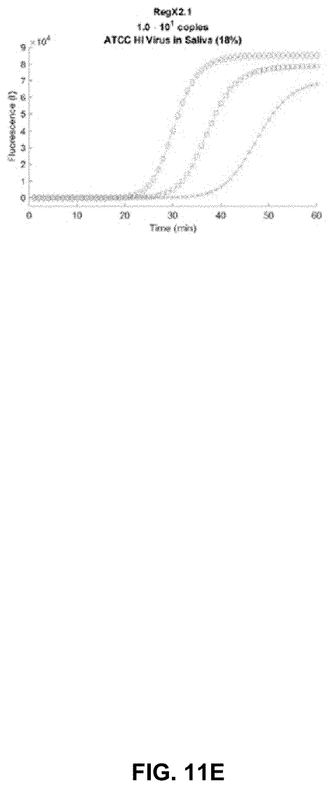

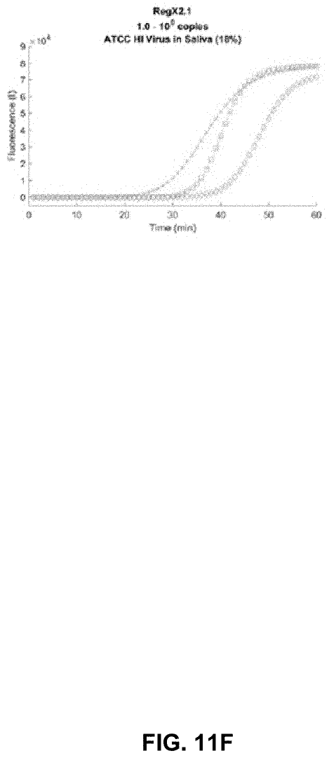

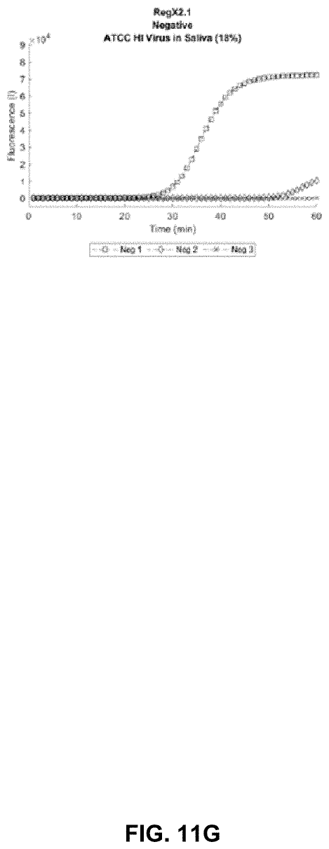

As illustrated in FIGS. 11A-11G, a graph of intensity of fluorescence over time in minutes was generated using 3 samples with a spiked SARS-CoV-2 virus in 18% saliva and 3 samples without the spiked SARS-CoV-2 virus in 18% saliva for the Region X2.1. Black lines indicate a non-template control (NTC), wherein 5 μL of saliva diluted to 18% in water was added to the reaction mix. Green lines indicate the samples spiked with SARS-CoV-2.

As shown in FIG. 11A, the three virus-spiked samples reach a fluorescence of about 7×10⁴ in intensity 20 minutes after commencement of the reaction for a viral concentration of about 100 k copies of the SARS-CoV-2 virus.

As illustrated in FIG. 11B, the three virus-spiked samples reached a fluorescence of about 7×10⁴ in intensity 20-30 minutes after commencement of the reaction for a viral concentration of about 10 k copies of the SARS-CoV-2 virus.

As illustrated in FIG. 11C, one of the three virus-spiked samples reached a fluorescence of about 7×10⁴ in intensity 30 minutes after commencement of the reaction for a viral concentration of about 1 k copies of the SARS-CoV-2 virus. The other two of the three virus-spiked samples exhibited a spike in fluorescence above the baseline level 40-60 minutes after commencement of the reaction.

As illustrated in FIG. 11D, all of the three virus-spiked samples reached a fluorescence of about 7×10⁴ in intensity 30-40 minutes after commencement of the reaction for a viral concentration of about 100 copies of the SARS-CoV-2 virus.

As illustrated in FIG. 11E, one of the three virus-spiked samples reached a fluorescence of about 7×10⁴ in intensity 30-60 minutes after commencement of the reaction for a viral concentration of about 10 copies of the SARS-CoV-2 virus.

As illustrated in FIG. 11F, all of the three virus-spiked samples reached a fluorescence of about 7×10⁴ in intensity 45-60 minutes after commencement of the reaction for a viral concentration of about 1 copy of the SARS-CoV-2 virus.

As illustrated in FIG. 11G, for the controls that were not spiked with SARS-CoV-2 virus, one of the three samples reached a fluorescence of about 7×10⁴ in intensity 45 minutes after commencement of the reaction. The other two of the three virus-spiked samples did not exhibit a spike in fluorescence until 50 minutes after commencement of the reaction.

Based on the data presented in FIGS. 11A-11G, the Region X2.1 primer set did not provide consistent results for detecting SARS-CoV-2 at varying concentrations of SARS-CoV-2 in comparison to the Reg X3.1 primer set.

### Example 14—Comparative Primer Set Performance—Region X3.1

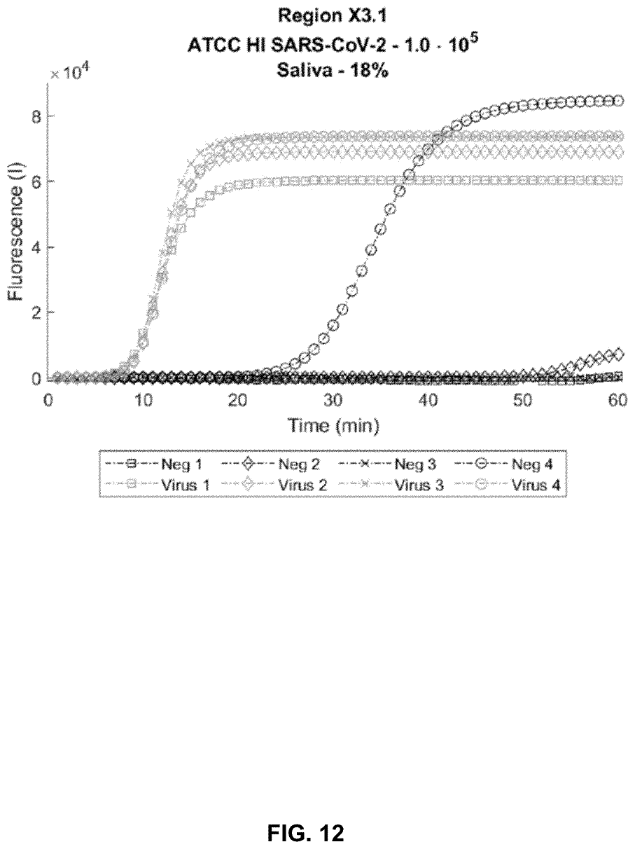

As illustrated in FIG. 12, a graph of intensity of fluorescence over time in minutes was generated using 4 samples with a spiked SARS-CoV-2 virus in 18% saliva and 4 samples without the spiked SARS-CoV-2 virus in 18% saliva for the Region X3.1. Black lines indicate a non-template control (NTC), wherein 5 μL of saliva diluted to 18% in water was added to the reaction mix. Green lines indicate the samples spiked with SARS-CoV-2.

The four virus-spiked samples reach a fluorescence of about 7×10⁴ in intensity 15 minutes after commencement of the reaction for a viral concentration of about 100 k copies of the SARS-CoV-2 virus. One of the four controls spiked after about 40 minutes with the remaining three controls exhibiting no spike in fluorescence above a baseline level.

### Example 15—Comparative Primer Set Performance—Region X3.1

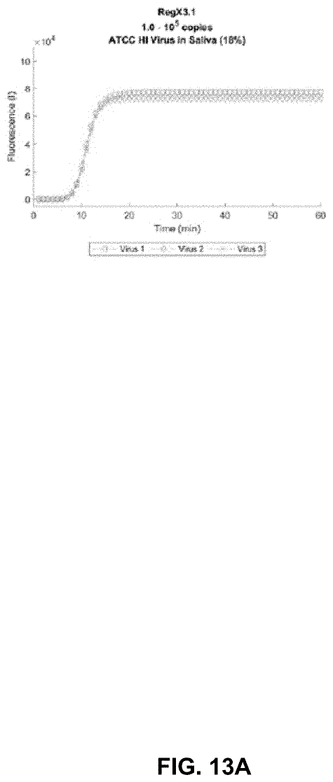

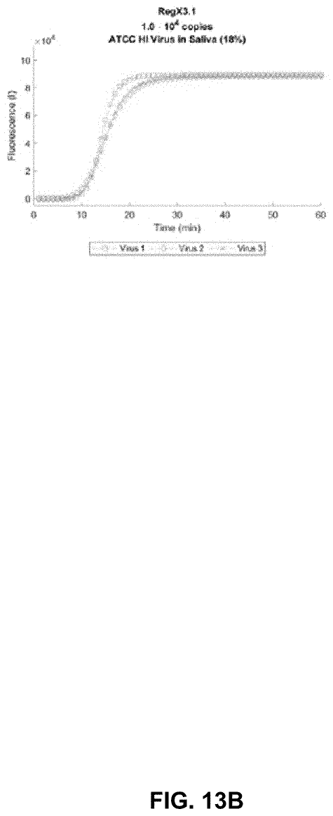

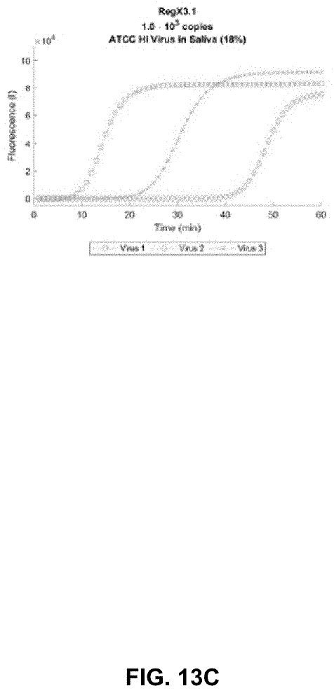

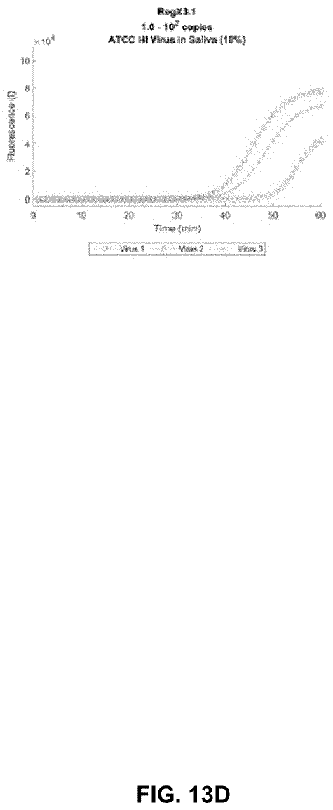

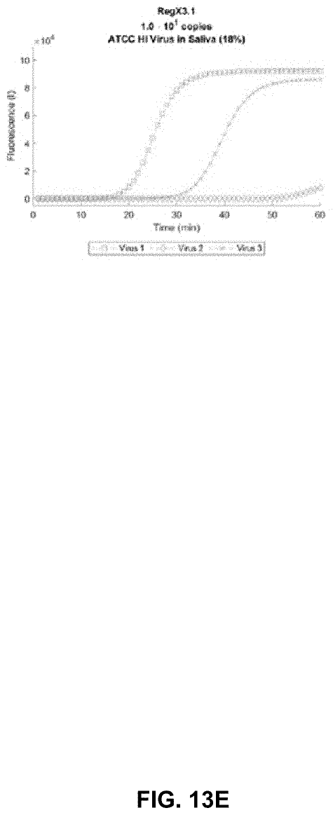

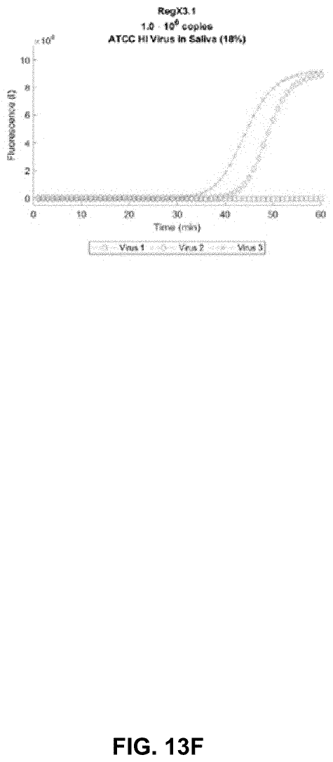

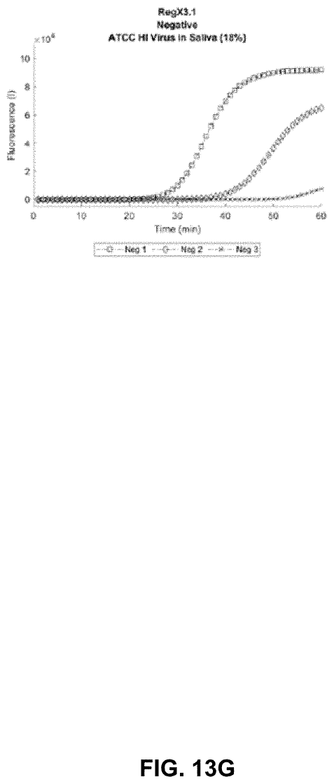

As illustrated in FIGS. 13A-13G, a graph of intensity of fluorescence over time in minutes was generated using 3 samples with a spiked SARS-CoV-2 virus in 18% saliva and 3 samples without the spiked SARS-CoV-2 virus in 18% saliva for the Region X2.1. Black lines indicate a non-template control (NTC), wherein 5 μL of saliva diluted to 18% in water was added to the reaction mix. Green lines indicate the samples spiked with SARS-CoV-2.

As shown in FIG. 13A, the three virus-spiked samples reach a fluorescence of about 7×10⁴ in intensity 10 minutes after commencement of the reaction for a viral concentration of about 100 k copies of the SARS-CoV-2 virus.

As illustrated in FIG. 13B, the three virus-spiked samples reached a fluorescence of about 7×10⁴ in intensity 20 minutes after commencement of the reaction for a viral concentration of about 10 k copies of the SARS-CoV-2 virus.

As illustrated in FIG. 13C, one of the three virus-spiked samples reached a fluorescence of about 7×10⁴ in intensity 20 minutes after commencement of the reaction for a viral concentration of about 1 k copies of the SARS-CoV-2 virus. The other two of the three virus-spiked samples exhibited a spike in fluorescence above the baseline level 40-60 minutes after commencement of the reaction.

As illustrated in FIG. 13D, all of the three virus-spiked samples reached a fluorescence of about 7×10⁴ in intensity 50 minutes after commencement of the reaction for a viral concentration of about 100 copies of the SARS-CoV-2 virus.

As illustrated in FIG. 13E, one of the three virus-spiked samples reached a fluorescence of about 7×10⁴ in intensity 30 minutes after commencement of the reaction for a viral concentration of about 10 copies of the SARS-CoV-2 virus. Another one of the three virus-spiked samples reached a fluorescence of about 7×10⁴ in intensity 45 minutes after commencement of the reaction for a viral concentration of about 10 copies of the SARS-CoV-2 virus. Another one of the three virus-spiked samples did not exhibit a spike in fluorescent above the baseline level.

As illustrated in FIG. 13F, two of the three virus-spiked samples reached a fluorescence of about 7×10⁴ in intensity 45-60 minutes after commencement of the reaction for a viral concentration of about 1 copy of the SARS-CoV-2 virus. Another one of the three virus-spiked samples did not exhibit a spike in fluorescent above the baseline level.

As illustrated in FIG. 13G, for the controls that were not spiked with SARS-CoV-2 virus, one of the three samples reached a fluorescence of about 7×10⁴ in intensity 40 minutes after commencement of the reaction. Another one of the three virus-spiked samples did not exhibit a spike in fluorescence until 50 minutes after commencement of the reaction. Another one of the three virus-spiked samples did not exhibit a spike in fluorescent above the baseline level.

Based on the data presented in FIGS. 13A-13G, the Region X3.1 primer set provided performance results that were more reliable, accurate, and consistent in comparison to the other primer sets (e.g., REG X1.1, REG X1.2, REG X2.1, REG X2.2, REG X2.3, REG X2.4, Orflab0.2).

### Example 16—Comparative Primer Set Performance—Orflab.2

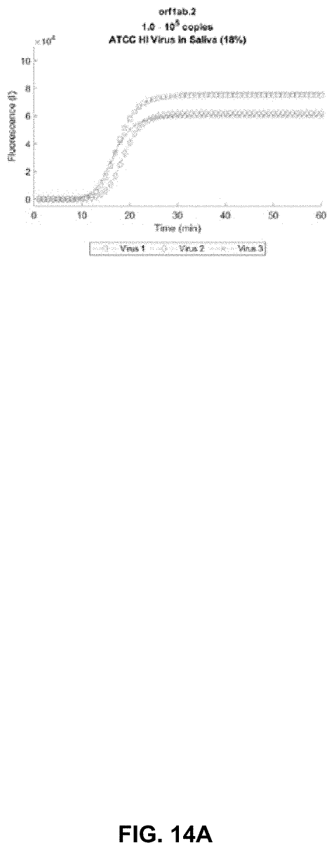

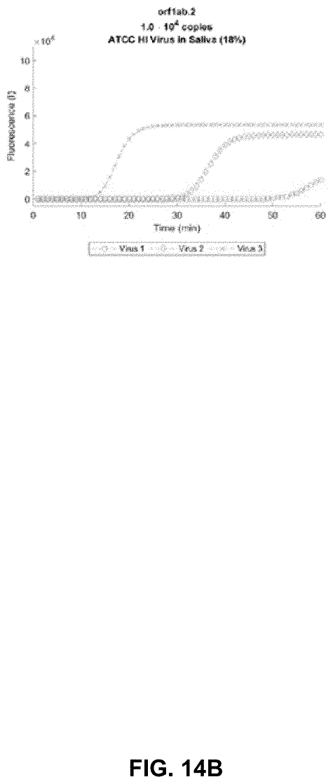

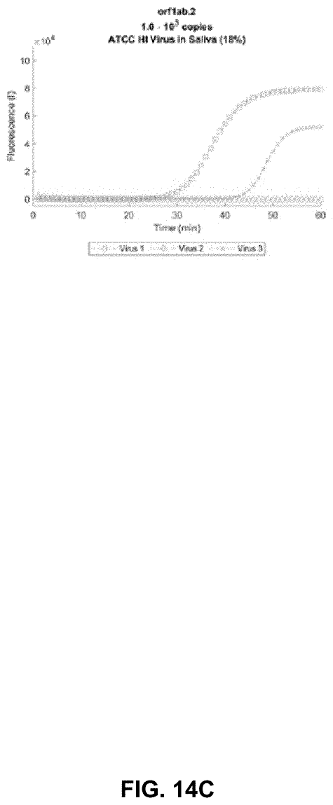

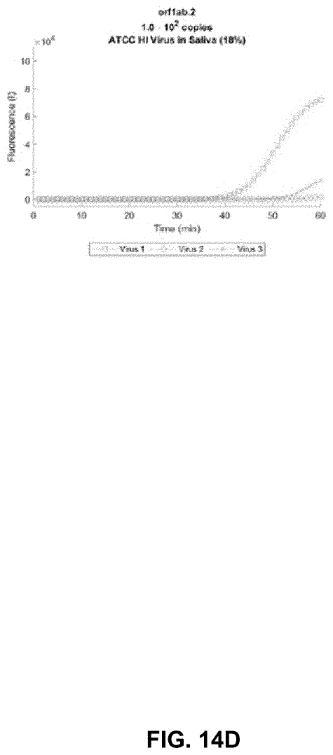

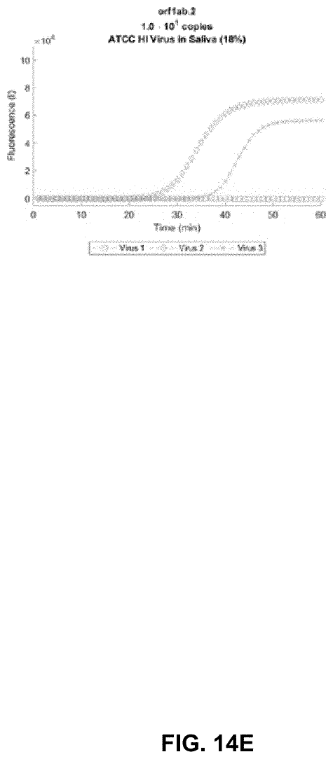

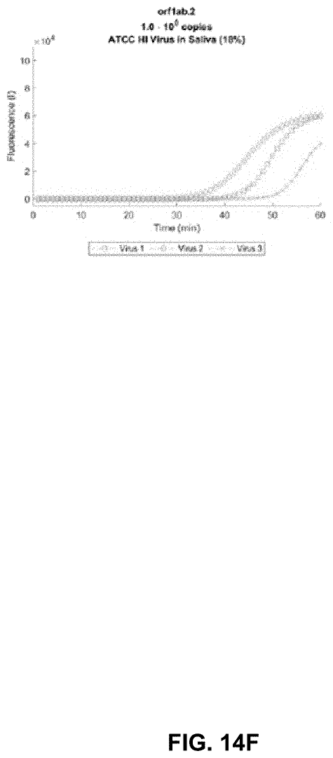

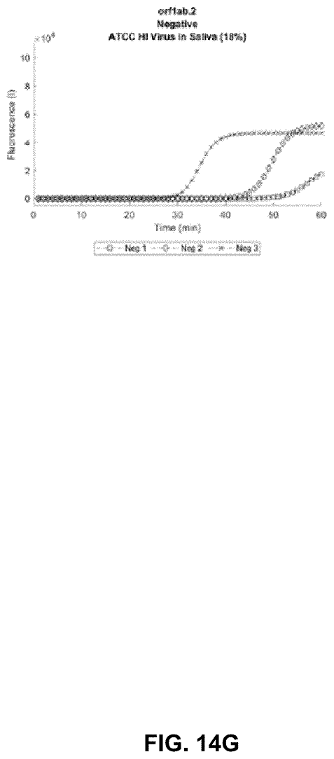

As illustrated in FIGS. 14A-14G, a graph of intensity of fluorescence over time in minutes was generated using 3 samples with a spiked SARS-CoV-2 virus in 18% saliva and 3 samples without the spiked SARS-CoV-2 virus in 18% saliva for the Region Orflab.2. Black lines indicate a non-template control (NTC), wherein 5 μL of saliva diluted to 18% in water was added to the reaction mix. Green lines indicate the samples spiked with SARS-CoV-2.

As shown in FIG. 14A, the three virus-spiked samples reach a fluorescence of about 7×10⁴ in intensity 20 minutes after commencement of the reaction for a viral concentration of about 100 k copies of the SARS-CoV-2 virus.

As illustrated in FIG. 14B, the three virus-spiked samples reached a fluorescence of about 6×10⁴ in intensity 20 minutes after commencement of the reaction for a viral concentration of about 10 k copies of the SARS-CoV-2 virus. The other two of the three virus-spiked samples exhibited a spike in fluorescence above the baseline level 40-60 minutes after commencement of the reaction.

As illustrated in FIG. 14C, one of the three virus-spiked samples reached a fluorescence of about 8×10⁴ in intensity 40 minutes after commencement of the reaction for a viral concentration of about 1 k copies of the SARS-CoV-2 virus. Another one of the three virus-spiked samples exhibited a spike in fluorescence above the baseline level 40-60 minutes after commencement of the reaction. Another one of the three virus-spiked samples did not exhibit a spike in fluorescent above the baseline level.

As illustrated in FIG. 14D, one of the three virus-spiked samples reached a fluorescence of about 7×10⁴ in intensity 60 minutes after commencement of the reaction for a viral concentration of about 100 copies of the SARS-CoV-2 virus. The other two of the three viral-spiked samples did not exhibit a spike in fluorescent above the baseline level until after 60 minutes.

As illustrated in FIG. 14E, one of the three virus-spiked samples reached a fluorescence of about 7×10⁴ in intensity 40 minutes after commencement of the reaction for a viral concentration of about 10 copies of the SARS-CoV-2 virus. Another one of the three virus-spiked samples reached a fluorescence of about 6×10⁴ in intensity 50 minutes after commencement of the reaction for a viral concentration of about 10 copies of the SARS-CoV-2 virus. Another one of the three virus-spiked samples did not exhibit a spike in fluorescent above the baseline level.

As illustrated in FIG. 14F, the three virus-spiked samples reached a fluorescence of about 4×10⁴ in intensity 60 minutes after commencement of the reaction for a viral concentration of about 1 copy of the SARS-CoV-2 virus.

As illustrated in FIG. 14G, for the controls that were not spiked with SARS-CoV-2 virus, one of the three samples reached a fluorescence of about 4×10⁴ in intensity 35 minutes after commencement of the reaction. Another one of the three virus-spiked samples did not exhibit a spike in fluorescence until 50 minutes after commencement of the reaction. Another one of the three virus-spiked samples did not exhibit a spike in fluorescent above the baseline level.

Based on the data presented in FIGS. 14A-14G, the Region Orflab.2 primer sets did not provide consistent results for detecting SARS-CoV-2 at varying concentrations of SARS-CoV-2 in comparison to the Reg X3.1 primer set.

### Example 17—List of Primers with Reverse Complements

A list of primers (F3, B3, FIP, BIP, LF, and LB) with sequences and reverse complements for N.3, N.6, N.10, N.13e, RdRP.1, RdRP.2, RdRP.3, RdRP.4, orflab.1, orflab.2, orflab.3, orflab.4, E.1, E.2, E.3, E.4, E.5, RNaseP.1, RNaseP.2, RNaseP.3, RNaseP.4, RNaseP.5, RegX1.1, RegX1.2, RegX2.1, RegX2.2, RegX2.3, RegX2.4, RegX2.3, RegX2.4, and RegX3.1 can be found in Table 10.

TABLE 10
List of primer sequences and reverse complements

| Primer | Sequence | Reverse Complement |
|---|---|---|
| N.3_F3 | TGGACCCCAAAATCAGCG | CGCTGATTTTGGGGTCCA |
| N.3_B3 | GCCTTGTCCTCGAGGGAAT | ATTCCCTCGAGGACAAGGC |
| N.3_FIP | CCACTGCGTTCTCCATTCTGGTA | CGTAATGCGGGGTGCATTTACCAG |
| | AATGCACCCCGCATTACG | AATGGAGAACGCAGTGG |
| N.3_BIP | CGCGATCAAAACAACGTCGGCC | TCTCACTCAACATGGCAAGGGCCG |
| | CTTGCCATGTTGAGTGAGA | ACGTTGTTTTGATCGCG |
| N.3_LF | GTTGAATCTGAGGGTCCACCA | TGGTGGACCCTCAGATTCAAC |
| N.3_LB | ACCCAATAATACTGCGTCTTGG | CCAAGACGCAGTATTATTGGGT |
| N.6_F3 | CCCCAAAATCAGCGAAATGC | GCATTTCGCTGATTTTGGGG |
| N.6_B3 | AGCCAATTTGGTCATCTGGA | TCCAGATGACCAAATTGGCT |
| N.6_FIP | CGACGTTGTTTTGATCGCGCCA | GAGGGTCCACCAAACGTAATGGC |
| | TTACGTTTGGTGGACCCTC | GCGATCAAAACAACGTCG |
| N.6_BIP | GCGTCTTGGTTCACCGCTCTAA | GAGGACAAGGCGTTCCAATTAGA |
| | TTGGAACGCCTTGTCCTC | GCGGTGAACCAAGACGC |
| N.6_LF | TCCATTCTGGTTACTGCCAGTTG | CAACTGGCAGTAACCAGAATGGA |
| N.6_LB | CAACATGGCAAGGAAGACCTT | AAGGTCTTCCTTGCCATGTTG |
| N.10_F3 | GCCCCAAGGTTTACCCAAT | ATTGGGTAAACCTTGGGGC |
| N.10_B3 | AGCACCATAGGGAAGTCCAG | CTGGACTTCCCTATGGTGCT |
| N.10_FIP | CGCCTTGTCCTCGAGGGAATTC | GAGCGGTGAACCAAGACGAATTC |
| | GTCTTGGTTCACCGCTC | CCTCGAGGACAAGGCG |
| N.10_BIP | AGACGAATTCGTGGTGGTGACG | CTACCTAGGAACTGGGCCACGTCA |
| | TGGCCCAGTTCCTAGGTAG | CCACCACGAATTCGTCT |
| N.10_LF | TCTTCCTTGCCATGTTGAGTG | CACTCAACATGGCAAGGAAGA |
| N.10_LB | ATGAAAGATCTCAGTCCAAGAT | CCATCTTGGACTGAGATCTTTCAT |
| | GG | |
| N.13e_F3 | AATTGGCTACTACCGAAGAGCT | TAGCTCTTCGGTAGTAGCCAATT |
| | A | |
| N.13e_B3 | GTAGAAGCCTTTTGGCAATGTT | CAACATTGCCAAAAGGCTTCTAC |
| | G | |
| N.13e_FIP | GTCTTTGTTAGCACCATAGGGA | CCATCTTGGACTGAGATCTTTCAG |
| | AGTCCTGAAAGATCTCAGTCCA | GACTTCCCTATGGTGCTAACAAAG |
| | AGATGG | AC |
| N.13e_BIP | GGAGCCTTGAATACACCAAAAG | CAATCGTGCTACAACTTCCTCAAG |
| | ATCACTTGAGGAAGTTGTAGCA | TGATCTTTTGGTGTATTCAAGGCTC |
| | CGATTG | C |
| N.13e_LF | TGGCCCAGTTCCTAGGTAGTAG | TATTTCTACTACCTAGGAACTGGG |
| | AAATA | CCA |
| N.13e_LB | CGCAATCCTGCTAACAATGCTG | CAGCATTGTTAGCAGGATTGCG |
| RdRP.1_ | CAGATGCATTCTGCATTGT | ACAATGCAGAATGCATCTG |
| F3 | | |
| RdRP.1_ | ATTACCAGAAGCAGCGTG | CACGCTGCTTCTGGTAAT |
| B3 | | |
| RdRP.1_ | CAGTTGAAACTACAAATGGAAC | TGTAGGTGGGAACACTGTAGGTGT |
| FIP | ACCTACAGTGTTCCCACCTACA | TCCATTTGTAGTTTCAACTG |
| RdRP.1_ | AGCTAGGTGTTGTACATAATCA | CTTGTGTATGCTGCTGACCTCCTG |
| BIP | GGAGGTCAGCAGCATACACAA | ATTATGTACAACACCTAGCT |
| | G | |
| RdRP.1_ | TTTTCTCACTAGTGGTCCAAAA | AGTTTTGGACCACTAGTGAGAAAA |
| LF | CT | |
| RdRP.1_ | TGTAAACTTACATAGCTCTAGA | AAGTCTAGAGCTATGTAAGTTTAC |
| LB | CTT | A |
| RdRP.2_ | ACTATGACCAATAGACAGTTTC | TGAAACTGTCTATTGGTCATAGT |
| F3 | A | |
| RdRP.2_ | GGCCATAATTCTAAGCATGTT | AACATGCTTAGAATTATGGCC |
| B3 | | |
| RdRP.2_ | GCCAACCACCATAGAATTTGCT | CCTCTAGTGGCGGCTATTAGCAAA |
| FIP | AATAGCCGCCACTAGAGG | TTCTATGGTGGTTGGC |
| RdRP.2_ | AGTGATGTAGAAAACCCTCACC | ATGTGATAGAGCCATGCCTAGGTG |
| BIP | TAGGCATGGCTCTATCACAT | AGGGTTTTCTACATCACT |
| RdRP.2_ | GTTCCAATTACTACAGTAGC | GCTACTGTAGTAATTGGAAC |
| LF | | |
| RdRP.2_ | ATGGGTTGGGATTATCCTAA | TTAGGATAATCCCAACCCAT |
| LB | | |
| RdRP.3_ | GCAAAATGTTGGACTGAGAC | GTCTCAGTCCAACATTTTGC |
| F3 | | |
| RdRP.3_ | GAACCGTTCAATCATAAGTGTA | TACACTTATGATTGAACGGTTC |
| B3 | | |
| RdRP.3_ | ATCACCCTGTTTAACTAGCATT | GAGGTCCTTTAGTAAGGTCAACAA |
| FIP | GTTGACCTTACTAAAGGACCTC | TGCTAGTTAAACAGGGTGAT |
| RdRP.3_ | TATGTGTACCTTCCTTACCCAG | AGATGATATCGTAAAAACAGATG |
| BIP | ACCATCTGTTTTTACGATATCAT | GTCTGGGTAAGGAAGGTACACATA |
| | CT | |
| RdRP.3_ | ATGTTGAGAGCAAAATTCAT | ATGAATTTTGCTCTCAACAT |
| LF | | |
| RdRP.3_ | TCCATCAAGAATCCTAGGGGC | GCCCCTAGGATTCTTGATGGA |
| LB | | |
| RdRP.4_ | CGATAAGTATGTCCGCAATT | AATTGCGGACATACTTATCG |
| F3 | | |
| RdRP.4_ | ACTGACTTAAAGTTCTTTATGCT | AGCATAAAGAACTTTAAGTCAGT |
| B3 | | |
| RdRP.4_ | ATGCGTAAAACTCATTCACAAA | GACACTCATAAAGTCTGTGTTGGA |
| FIP | GTCCAACACAGACTTTATGAGT | CTTTGTGAATGAGTTTTACGCAT |
| | GTC | |
| RdRP.4_ | TGATACTCTCTGACGATGCTGT | ATCTCAAGGTCTAGTGGCTACAGC |
| BIP | AGCCACTAGACCTTGAGAT | ATCGTCAGAGAGTATCA |
| RdRP.4_ | TGTGTCAACATCTCTATTTCTAT | CTATAGAAATAGAGATGTTGACAC |
| LF | AG | A |
| RdRP.4_ | TGTGTGTTTCAATAGCACTTAT | GCATAAGTGCTATTGAAACACACA |
| LB | GC | |
| orf1ab.1_ | AGCTGGTAATGCAACAGAA | TTCTGTTGCATTACCAGCT |
| F3 | | |
| orf1ab.1_ | CACCACCAAAGGATTCTTG | CAAGAATCCTTTGGTGGTG |
| B3 | | |
| orf1ab.1_ | TCCCCCACTAGCTAGATAATCT | CAGAAAGATAATACAGTTGAATTG |
| FIP | TTGCCAATTCAACTGTATTATCT | GCAAAGATTATCTAGCTAGTGGGG |
| | TTCTG | GA |
| orf1ab.1_ | GTGTTAAGATGTTGTGTACACA | CCGGAAGCCAATATGGATGTGTGT |
| BIP | CACATCCATATTGGCTTCCGG | GTACACAACATCTTAACAC |
| orf1ab.1_ | GCTTTAGCAGCATCTACAGCA | TGCTGTAGATGCTGCTAAAGC |
| LF | | |
| orf1ab.1_ | TGGTACTGGTCAGGCAATAACA | ACTGTTATTGCCTGACCAGTACCA |
| LB | GT | |
| orf1ab.2_ | ACTTAAAAACACAGTCTGTACC | GGTACAGACTGTGTTTTTAAGT |
| F3 | | |
| orf1ab.2_ | TCAAAAGCCCTGTATACGA | TCGTATACAGGGCTTTTGA |
| B3 | | |
| orf1ab.2_ | TGACTGAAGCATGGGTTCGCGT | CTTTCCACATACCGCAGACGCGAA |
| FIP | CTGCGGTATGTGGAAAG | CCCATGCTTCAGTCA |
| orf1ab.2_ | GCTGATGCACAATCGTTTTTAA | ACAGGCACTAGTACTGATGCGTTT |
| BIP | ACGCATCAGTACTAGTGCCTGT | AAAAACGATTGTGCATCAGC |
| orf1ab.2_ | GAGTTGATCACAACTACAGCCA | TATGGCTGTAGTTGTGATCAACTC |
| LF | TA | |
| orf1ab.2_ | TTGCGGTGTAAGTGCAGCC | GGCTGCACTTACACCGCAA |
| LB | | |
| orf1ab.3_ | TTGTGCTAATGACCCTGT | ACAGGGTCATTAGCACAA |
| F3 | | |
| orf1ab.3_ | TCAAAAGCCCTGTATACGA | TCGTATACAGGGCTTTTGA |
| B3 | | |
| orf1ab.3_ | GATCACAACTACAGCCATAACC | CTGTGTTTTTAAGTGTAAAACCCA |
| FIP | TTTGGGTTTTACACTTAAAAAC | AAGGTTATGGCTGTAGTTGTGATC |
| | ACAG | |
| orf1ab.3_ | TGATGCACAATCGTTTTTAAAC | ACAGGCACTAGTACTGATGCCGTT |
| BIP | GGCATCAGTACTAGTGCCTGT | TAAAAACGATTGTGCATCA |
| orf1ab.3_ | CCACATACCGCAGACGGTACAG | CTGTACCGTCTGCGGTATGTGG |
| LF | | |
| orf1ab.3_ | GGTGTAAGTGCAGCCCGT | ACGGGCTGCACTTACACC |
| LB | | |
| orf1ab.4_ | CTGTTATCCGATTTACAGGATT | AATCCTGTAAATCGGATAACAG |
| F3 | | |
| orf1ab.4_ | GGCAGCTAAACTACCAAGT | ACTTGGTAGTTTAGCTGCC |
| B3 | | |
| orf1ab.4_ | ACAAGGTGGTTCCAGTTCTGTA | ACTCTTAGGGAATCTAGCCCTACA |
| FIP | GGGCTAGATTCCCTAAGAGT | GAACTGGAACCACCTTGT |
| orf1ab.4_ | TGTTACAGACACACCTAAAGGT | AACAACCTAAATAGAGGTATGGTG |
| BIP | CCACCATACCTCTATTTAGGTT | GACCTTTAGGTGTGTCTGTAACA |
| | GTT | |
| orf1ab.4_ | TAGATAGTACCAGTTCCATC | GATGGAACTGGTACTATCTA |
| LF | | |
| orf1ab.4_ | TGAAGTATTTATACTTTATTAA | CCTTTAATAAAGTATAAATACTTC |
| LB | AGG | A |
| E.1_F3 | CCTGAAGAACATGTCCAAAT | ATTTGGACATGTTCTTCAGG |
| E.1_B3 | CGCTATTAACTATTAACGTACC | AGGTACGTTAATAGTTAATAGCG |
| | T | |
| E.1_FIP | CGTCGGTTCATCATAAATTGGT | GGATGAACCGTCGATTGTGGAACC |
| | TCCACAATCGACGGTTCATCC | AATTTATGATGAACCGACG |
| E.1_BIP | ACTACTAGCGTGCCTTTGTAAG | CATTCGTTTCGGAAGAGACGCTTA |
| | CGTCTCTTCCGAAACGAATG | CAAAGGCACGCTAGTAGT |
| E.1_LF | CATTACTGGATTAACAACTCC | GGAGTTGTTAATCCAGTAATG |
| E.1_LB | ACAAGCTGATGAGTACGAACTT | CATAAGTTCGTACTCATCAGCTTG |
| | ATG | T |
| E.2_F3 | TTGTAAGCACAAGCTGATG | CATCAGCTTGTGCTTACAA |
| E.2_B3 | AGAGTAAACGTAAAAAGAAGG | AACCTTCTTTTTACGTTTACTCT |
| | TT | |
| E.2_FIP | CGAAAGCAAGAAAAAGAAGTA | CGAATGAGTACATAAGTTCGTACT |
| | CGCTAGTACGAACTTATGTACT | AGCGTACTTCTTTTTCTTGCTTTCG |
| | CATTCG | |
| E.2_BIP | TGGTATTCTTGCTAGTTACACTA | GCAATATTGTTAACGTGAGTCTGC |
| | GCAGACTCACGTTAACAATATT | TAGTGTAACTAGCAAGAATACCA |
| | GC | |
| E.2_LF | ACGTACCTGTCTCTTCCGAAA | TTTCGGAAGAGACAGGTACGT |
| E.2_LB | CATCCTTACTGCGCTTCGATTGT | CACAATCGAAGCGCAGTAAGGAT |
| | G | G |
| E.3_F3 | GTACGAACTTATGTACTCATTC | CGAATGAGTACATAAGTTCGTAC |
| | G | |
| E.3_B3 | TTTTTAACACGAGAGTAAACGT | ACGTTTACTCTCGTGTTAAAAA |
| E.3_FIP | CTAGCAAGAATACCACGAAAG | CGTACCTGTCTCTTCCGAACTTGCT |
| | CAAGTTCGGAAGAGACAGGTA | TTCGTGGTATTCTTGCTAG |
| | CG | |
| E.3_BIP | CACTAGCCATCCTTACTGCGCA | ACGTGAGTCTTGTAAAACCTTGCG |
| | AGGTTTTACAAGACTCACGT | CAGTAAGGATGGCTAGTG |
| E.3_LF | AGAAGTACGCTATTAACTATTA | TAATAGTTAATAGCGTACTTCT |
| E.3_LB | TTCGATTGTGTGCGTACTGCTG | CAGCAGTACGCACACAATCGAA |
| E.4_F3 | CACTAGCCATCCTTACTGC | GCAGTAAGGATGGCTAGTG |
| E.4_B3 | GTACCGTTGGAATCTGCC | GGCAGATTCCAACGGTAC |
| E.4_FIP | ACGAGAGTAAACGTAAAAAGA | TACGCACACAATCGAAGCACCTTC |
| | AGGTGCTTCGATTGTGTGCGTA | TTTTTACGTTTACTCTCGT |
| E.4_BIP | CTAGAGTTCCTGATCTTCTGGTC | GTTTGGAACTTTAATTTTAGCCAA |
| | TTGGCTAAAATTAAAGTTCCAA | GACCAGAAGATCAGGAACTCTAG |
| | AC | |
| E.4_LF | AGACTCACGTTAACAATATTGC | GCTGCAATATTGTTAACGTGAGTC |
| | AGC | T |
| E.4_LB | ACGAACTAAATATTATATTAGT | AAAACTAATATAATATTTAGTTCG |
| | TTT | T |
| E.5_F3 | ACTCTCGTGTTAAAAATCTGAA | TTCAGATTTTTAACACGAGAGT |
| E.5_B3 | GCAAATTGTAGAAGACAAATCC | ATGGATTTGTCTTCTACAATTTGC |
| | AT | |
| E.5_FIP | CTGCCATGGCTAAAATTAAAGT | AGACCAGAAGATCAGGAACTGGA |
| | TCCAGTTCCTGATCTTCTGGTCT | ACTTTAATTTTAGCCATGGCAG |
| E.5_BIP | TCCAACGGTACTATTACCGTTG | CCTAGTAATAGGTTTCCTATTCCTT |
| | AAAGGAATAGGAAACCTATTAC | TCAACGGTAATAGTACCGTTGGA |
| | TAGG | |
| E.5_LF | AAAACTAATATAATATTTAGTT | ACGAACTAAATATTATATTAGTTT |
| | CGT | T |
| E.5_LB | AAAAAGCTCCTTGAACAATGGA | TTCCATTGTTCAAGGAGCTTTTT |
| | A | |
| RNaseP.1_ | GGTGGCTGCCAATACCTC | GAGGTATTGGCAGCCACC |
| F3 | | |
| RNaseP.1_ | ACTCAGCATGCGAAGAGC | GCTCTTCGCATGCTGAGT |
| B3 | | |
| RNaseP.1_ | GTTGCGGATCCGAGTCAGTGGC | TCATCAACAAGCTCCACGGCCACT |
| FIP | CGTGGAGCTTGTTGATGA | GACTCGGATCCGCAAC |
| RNaseP.1_ | AACTCAGCCATCCACATCCGAG | CCACTTATCCCCTCCGTGACTCGG |
| BIP | TCACGGAGGGGATAAGTGG | ATGTGGATGGCTGAGTT |
| RNaseP.1_ | GTGTGTCGGTCTCTGGCTCCA | TGGAGCCAGAGACCGACACAC |
| LF | | |
| RNaseP.1_ | TCTTCAGGGTCACACCCAAGT | ACTTGGGTGTGACCCTGAAGA |
| LB | | |
| RNaseP.2_ | CGTGGAGCTTGTTGATGAGC | GCTCATCAACAAGCTCCACG |
| F3 | | |
| RNaseP.2_ | TGGGCTTCCAGGGAACAG | CTGTTCCCTGGAAGCCCA |
| B3 | | |
| RNaseP.2_ | CGGATGTGGATGGCTGAGTTGT | TGTGTCGGTCTCTGGCTCACAACT |
| FIP | GAGCCAGAGACCGACACA | CAGCCATCCACATCCG |
| RNaseP.2_ | ACTCCTCCACTTATCCCCTCCGT | GTACTGGACCTCGGACCACGGAGG |
| BIP | GGTCCGAGGTCCAGTAC | GGATAAGTGGAGGAGT |
| RNaseP.2_ | ATCCGAGTCAGTGGCTCCCG | CGGGAGCCACTGACTCGGAT |
| LF | | |
| RNaseP.2_ | ATATGGCTCTTCGCATGCTG | CAGCATGCGAAGAGCCATAT |
| LB | | |
| RNaseP.3_ | TCAGGGTCACACCCAAGT | ACTTGGGTGTGACCCTGA |
| F3 | | |
| RNaseP.3_ | CGCATACACACACTCAGGAA | TTCCTGAGTGTGTGTATGCG |
| B3 | | |
| RNaseP.3_ | ACATGGCTCTGGTCCGAGGTCC | CACGGAGGGGATAAGTGGAGGAC |
| FIP | TCCACTTATCCCCTCCGTG | CTCGGACCAGAGCCATGT |
| RNaseP.3_ | CTGTTCCCTGGAAGCCCAAAGG | CTCTTGGTGGGCCCAGTTACCTTT |
| BIP | TAACTGGGCCCACCAAGAG | GGGCTTCCAGGGAACAG |
| RNaseP.3_ | ACTCAGCATGCGAAGAGCCATA | ATATGGCTCTTCGCATGCTGAGT |
| LF | T | |
| RNaseP.3_ | CTGCATTGAGGGTGGGGGTAAT | ATTACCCCCACCCTCAATGCAG |
| LB | | |
| RNaseP.4_ | GCCCTGTGGAACGAAGAG | CTCTTCGTTCCACAGGGC |
| F3 | | |
| RNaseP.4_ | TCCGTCCAGCAGCTTCTG | CAGAAGCTGCTGGACGGA |
| B3 | | |
| RNaseP.4_ | CACTGGATCCAGTTCAGCCTCC | TTCTGCCATGCTGTGTGCGGAGGC |
| FIP | GCACACAGCATGGCAGAA | TGAACTGGATCCAGTG |
| RNaseP.4_ | TTAGGAAAAGGCTTCCCAGCCG | AAGACGGACTTTAAGGCCCACGGC |
| BIP | TGGGCCTTAAAGTCCGTCTT | TGGGAAGCCTTTTCCTAA |
| RNaseP.4_ | CACCGCGGGGCTCTCGGT | ACCGAGAGCCCCGCGGTG |
| LF | | |
| RNaseP.4_ | CTGCCCCGGAGACCCAATG | CATTGGGTCTCCGGGGCAG |
| LB | | |
| RNaseP.5_ | TACATTCACGGCTTGGGC | GCCCAAGCCGTGAATGTA |
| F3 | | |
| RNaseP.5_ | GGGTGTGACCCTGAAGACT | AGTCTTCAGGGTCACACCC |
| B3 | | |
| RNaseP.5_ | CACCTGCAAGGACCCGAAGCA | GATGTTGATGGCGCGGTTGCTTCG |
| FIP | ACCGCGCCATCAACATC | GGTCCTTGCAGGTG |
| RNaseP.5_ | GCCAATACCTCCACCGTGGAGG | CTGACTCGGATCCGCAACCTCCAC |
| BIP | TTGCGGATCCGAGTCAG | GGTGGAGGTATTGGC |
| RNaseP.5_ | CGCCTGCAGCTGCAGCGC | GCGCTGCAGCTGCAGGCG |
| LF | | |
| RNaseP.5_ | GTTGATGAGCTGGAGCCAGAGA | TCTCTGGCTCCAGCTCATCAAC |
| LB | | |
| RegX1.1_ | GTCCGAACAACTGGACTT | AAGTCCAGTTGTTCGGAC |
| F3 | | |
| RegX1.1_ | GTCTTGATTATGGAATTTAAGG | TTCCCTTAAATTCCATAATCAAGA |
| B3 | GAA | C |
| RegX1.1_ | TTCCGTGTACCAAGCAATTTCA | TACACCCCTCTTAGTGTCACATGA |
| FIP | TGTGACACTAAGAGGGGTGTA | AATTGCTTGGTACACGGAA |
| RegX1.1_ | AAGAGCTATGAATTGCAGACAC | CTTCAATGGGGAATGTCCAGGTGT |
| BIP | CTGGACATTCCCCATTGAAG | CTGCAATTCATAGCTCTT |
| RegX1.1_ | CTCATGTTCACGGCAGCAGTA | TACTGCTGCCGTGAACATGAG |
| LF | | |
| RegX1.1_ | ATTGGCAAAGAAATTTGACAC | GTGTCAAATTTCTTTGCCAAT |
| LB | | |
| RegX1.2_ | GTCCGAACAACTGGACTT | AAGTCCAGTTGTTCGGAC |
| F3 | | |
| RegX1.2_ | GTCTTGATTATGGAATTTAAGG | TTCCCTTAAATTCCATAATCAAGA |
| B3 | GAA | C |
| RegX1.2_ | TTCCGTGTACCAAGCAATTTCA | TACACCCCTCTTAGTGTCACATGA |
| FIP | TGTGACACTAAGAGGGGTGTA | AATTGCTTGGTACACGGAA |
| RegX1.2_ | CTGAAAAGAGCTATGAATTGCA | TCAATGGGGAATGTCCAAGTCTGC |
| BIP | GACTTGGACATTCCCCATTGA | AATTCATAGCTCTTTTCAG |
| RegX1.2_ | TCATGTTCACGGCAGCAGTA | TACTGCTGCCGTGAACATGA |
| LF | | |
| RegX1.2_ | ATTGGCAAAGAAATTTGACACC | AGGTGTCAAATTTCTTTGCCAAT |
| LB | T | |
| RegX2.1_ | CTGTCCACGAGTGCTTTG | CAAAGCACTCGTGGACAG |
| F3 | | |
| RegX2.1_ | TGAGGTACACACTTAATAGCTT | AAGCTATTAAGTGTGTACCTCA |
| B3 | | |
| RegX2.1_ | AGCCGCATTAATCTTCAGTTCA | GTCCAGTCAACACGCTTAGATGAA |
| FIP | TCTAAGCGTGTTGACTGGAC | CTGAAGATTAATGCGGCT |
| RegX2.1_ | AGAAAGGTTCAACACATGGTTG | TCACGACATTGGTAACCCTAACAA |
| BIP | TTAGGGTTACCAATGTCGTGA | CCATGTGTTGAACCTTTCT |
| RegX2.1_ | ACCAATTATAGGATATTCAAT | ATTGAATATCCTATAATTGGT |
| LF | | |
| RegX2.1_ | AGCAGACAAATTCCCAGTTCT | AGAACTGGGAATTTGTCTGCT |
| LB | | |
| RegX2.2_ | CTGTCCACGAGTGCTTTG | CAAAGCACTCGTGGACAG |
| F3 | | |
| RegX2.2_ | TGAGGTACACACTTAATAGCT | AGCTATTAAGTGTGTACCTCA |
| B3 | | |
| RegX2.2_ | GCCGCATTAATCTTCAGTTCAT | TCCAGTCAACACGCTTAATGATGA |
| FIP | CATTAAGCGTGTTGACTGGA | ACTGAAGATTAATGCGGC |
| RegX2.2_ | AGAAAGGTTCAACACATGGTTG | ACGACATTGGTAACCCTAATAACA |
| BIP | TTATTAGGGTTACCAATGTCGT | ACCATGTGTTGAACCTTTCT |
| RegX2.2_ | CCAATTATAGGATATTCAATAG | CTATTGAATATCCTATAATTGG |
| LF | | |
| RegX2.2_ | TGCATTATTAGCAGACAAATTC | TGGGAATTTGTCTGCTAATAATGC |
| LB | CCA | A |
| RegX2.3_ | CTGTCCACGAGTGCTTTG | CAAAGCACTCGTGGACAG |
| F3 | | |
| RegX2.3_ | TGAGGTACACACTTAATAGCT | AGCTATTAAGTGTGTACCTCA |
| B3 | | |
| RegX2.3_ | GCCGCATTAATCTTCAGTTCAT | TCCAGTCAACACGCTTAATGATGA |
| FIP | CATTAAGCGTGTTGACTGGA | ACTGAAGATTAATGCGGC |
| RegX2.3_ | AGAAAGGTTCAACACATGGTTG | ACGACATTGGTAACCCTAAAACAA |
| BIP | TTTTAGGGTTACCAATGTCGT | CCATGTGTTGAACCTTTCT |
| RegX2.3_ | CCAATTATAGGATATTCAATAG | CTATTGAATATCCTATAATTGG |
| LF | | |
| RegX2.3_ | TGCATTATTAGCAGACAAATTC | TGGGAATTTGTCTGCTAATAATGC |
| LB | CCA | A |
| RegX2.4_ | CTGTCCACGAGTGCTTTG | CAAAGCACTCGTGGACAG |
| F3 | | |
| RegX2.4_ | TGAGGTACACACTTAATAGCT | AGCTATTAAGTGTGTACCTCA |
| B3 | | |
| RegX2.4_ | GCCGCATTAATCTTCAGTTCAT | GTCCAGTCAACACGCTTAATGATG |
| FIP | CATTAAGCGTGTTGACTGGAC | AACTGAAGATTAATGCGGC |
| RegX2.4_ | AGAAAGGTTCAACACATGGTTG | ACGACATTGGTAACCCTAAAACAA |
| BIP | TTTTAGGGTTACCAATGTCGT | CCATGTGTTGAACCTTTCT |
| RegX2.4_ | CCAATTATAGGATATTCAATA | TATTGAATATCCTATAATTGG |
| LF | | |
| RegX2.4_ | TGCATTATTAGCAGACAAATTC | TGGGAATTTGTCTGCTAATAATGC |
| LB | CCA | A |
| RegX3.1_ | CGGCGTAAAACACGTCTA | TAGACGTGTTTTACGCCG |
| F3 | | |
| RegX3.1_ | GCTAAAAAGCACAAATAGAAG | GACTTCTATTTGTGCTTTTTAGC |
| B3 | TC | |
| RegX3.1_ | GGAGAGTAAAGTTCTTGAACTT | CTGATCTGGCACGTAACTAGGAAG |
| FIP | CCTAGTTACGTGCCAGATCAG | TTCAAGAACTTTACTCTCC |
| RegX3.1_ | TGCGGCAATAGTGTTTATAACA | AGACAGAATGATTGAACTTTCATA |
| BIP | CTATGAAAGTTCAATCATTCTG | GTGTTATAAACACTATTGCCGCA |
| | TCT | |
| RegX3.1_ | TGTCTGATGAACAGTTTAGGTG | TTTCACCTAAACTGTTCATCAGAC |
| LF | AAA | A |
| RegX3.1_ | TTGCTTCACACTCAAAAGAA | TTCTTTTGAGTGTGAAGCAA |
| LB | | |

### Example 18—List of F2, F1c, B2, B1c Primers

A list of primers (F2, F1c, B2, and Bic) with forward sequences for N.3, N.6, N.10, N.13e, nsp12.1, nsp12.2, nsp12.3, nsp12.4, orflab.1, orflab.2, orflab.3, orflab.4, E.1, E.2, E.3, E.4, E.5, RNaseP.1, RNaseP.2, RNaseP.3, RNaseP.4, RNaseP.5, RegX1.1, RegX1.2, RegX2.1, RegX2.2, RegX2.3, RegX2.4, RegX2.3, RegX2.4, and RegX3.1 can be found in Table 11.

TABLE 11

| Sequence Name | Sequence (Forward) |
|---|---|
| SARS-CoV-2_N.3_F2 | AAATGCACCCCGCATTACG |
| SARS-CoV-2_N.3_F1C | CCACTGCGTTCTCCATTCTGGT |
| SARS-CoV-2_N.3_B2 | CCTTGCCATGTTGAGTGAGA |
| SARS-CoV-2_N.3_B1C | CGCGATCAAAACAACGTCGGC |
| SARS-CoV-2_N.6_F2 | ATTACGTTTGGTGGACCCTC |
| SARS-CoV-2_N.6_F1C | CGACGTTGTTTTGATCGCGCC |
| SARS-CoV-2_N.6_B2 | AATTGGAACGCCTTGTCCTC |
| SARS-CoV-2_N.6_B1C | GCGTCTTGGTTCACCGCTCT |
| SARS-CoV-2_N.10_F2 | CGTCTTGGTTCACCGCTC |
| SARS-CoV-2_N.10_F1C | CGCCTTGTCCTCGAGGGAATT |
| SARS-CoV-2_N.10_B2 | TGGCCCAGTTCCTAGGTAG |
| SARS-CoV-2_N.10_B1C | AGACGAATTCGTGGTGGTGACG |
| SARS-CoV-2_N.13e_F2 | TGAAAGATCTCAGTCCAAGATGG |
| SARS-CoV-2_N.13e_F1C | GTCTTTGTTAGCACCATAGGGAAGTCC |
| SARS-CoV-2_N.13e_B2 | TTGAGGAAGTTGTAGCACGATTG |
| SARS-CoV-2_N.13e_B1C | GGAGCCTTGAATACACCAAAAGATCAC |
| SARS-CoV-2_nsp12.1_F2 | TACAGTGTTCCCACCTACA |
| SARS-CoV-2_nsp12.1_F1C | CAGTTGAAACTACAAATGGAACACC |
| SARS-CoV-2_nsp12.1_B2 | GGTCAGCAGCATACACAAG |
| SARS-CoV-2_nsp12.1_B1C | AGCTAGGTGTTGTACATAATCAGGA |
| SARS-CoV-2_nsp12.2_F2 | AATAGCCGCCACTAGAGG |
| SARS-CoV-2_nsp12.2_F1C | GCCAACCACCATAGAATTTGCT |
| SARS-CoV-2_nsp12.2_B2 | AGGCATGGCTCTATCACAT |
| SARS-CoV-2_nsp12.2_B1C | AGTGATGTAGAAAACCCTCACCT |
| SARS-CoV-2_nsp12.3_F2 | TGACCTTACTAAAGGACCTC |
| SARS-CoV-2_nsp12.3_F1C | ATCACCCTGTTTAACTAGCATTGT |
| SARS-CoV-2_nsp12.3_B2 | CCATCTGTTTTTACGATATCATCT |
| SARS-CoV-2_nsp12.3_B1C | TATGTGTACCTTCCTTACCCAGA |
| SARS-CoV-2_nsp12.4_F2 | CAACACAGACTTTATGAGTGTC |
| SARS-CoV-2_nsp12.4_F1C | ATGCGTAAAACTCATTCACAAAGTC |
| SARS-CoV-2_nsp12.4_B2 | AGCCACTAGACCTTGAGAT |
| SARS-CoV-2_nsp12.4_B1C | TGATACTCTCTGACGATGCTGT |
| SARS-CoV-2_orf1ab.1_F2 | CCAATTCAACTGTATTATCTTTCTG |
| SARS-CoV-2_orf1ab.1_F1C | TCCCCCACTAGCTAGATAATCTTTG |
| SARS-CoV-2_orf1ab.1_B2 | ATCCATATTGGCTTCCGG |
| SARS-CoV-2_orf1ab.1_B1C | GTGTTAAGATGTTGTGTACACACAC |
| SARS-CoV-2_orf1ab.2_F2 | GTCTGCGGTATGTGGAAAG |
| SARS-CoV-2_orf1ab.2_F1C | TGACTGAAGCATGGGTTCGC |
| SARS-CoV-2_orf1ab.2_B2 | CATCAGTACTAGTGCCTGT |
| SARS-CoV-2_orf1ab.2_B1C | GCTGATGCACAATCGTTTTTAAACG |
| SARS-CoV-2_orf1ab.3_F2 | GGGTTTTACACTTAAAAACACAG |
| SARS-CoV-2_orf1ab.3_F1C | GATCACAACTACAGCCATAACCTTT |
| SARS-CoV-2_orf1ab.3_B2 | CATCAGTACTAGTGCCTGT |
| SARS-CoV-2_orf1ab.3_B1C | TGATGCACAATCGTTTTTAAACGG |
| SARS-CoV-2_orf1ab.4_F2 | GGGCTAGATTCCCTAAGAGT |
| SARS-CoV-2_orf1ab.4_F1C | ACAAGGTGGTTCCAGTTCTGTA |
| SARS-CoV-2_orf1ab.4_B2 | ACCATACCTCTATTTAGGTTGTT |
| SARS-CoV-2_orf1ab.4_B1C | TGTTACAGACACACCTAAAGGTCC |
| SARS-CoV-2_E.1_F2 | CACAATCGACGGTTCATCC |
| SARS-CoV-2_E.1_F1C | CGTCGGTTCATCATAAATTGGTTC |
| SARS-CoV-2_E.1_B2 | GTCTCTTCCGAAACGAATG |
| SARS-CoV-2_E.1_B1C | ACTACTAGCGTGCCTTTGTAAGC |
| SARS-CoV-2_E.2_F2 | AGTACGAACTTATGTACTCATTCG |
| SARS-CoV-2_E.2_F1C | CGAAAGCAAGAAAAAGAAGTACGCT |
| SARS-CoV-2_E.2_B2 | AGACTCACGTTAACAATATTGC |
| SARS-CoV-2_E.2_B1C | TGGTATTCTTGCTAGTTACACTAGC |
| SARS-CoV-2_E.3_F2 | TTCGGAAGAGACAGGTACG |
| SARS-CoV-2_E.3_F1C | CTAGCAAGAATACCACGAAAGCAAG |
| SARS-CoV-2_E.3_B2 | AAGGTTTTACAAGACTCACGT |
| SARS-CoV-2-E.3_B1C | CACTAGCCATCCTTACTGCGC |
| SARS-CoV-2_E.4_F2 | GCTTCGATTGTGTGCGTA |
| SARS-CoV-2_E.4_F1C | ACGAGAGTAAACGTAAAAAGAAGGT |
| SARS-CoV-2_E.4_B2 | TGGCTAAAATTAAAGTTCCAAAC |
| SARS-CoV-2_E.4_B1C | CTAGAGTTCCTGATCTTCTGGTCT |
| SARS-CoV-2_E.5_F2 | AGTTCCTGATCTTCTGGTCT |
| SARS-CoV-2_E.5_F1C | CTGCCATGGCTAAAATTAAAGTTCC |
| SARS-CoV-2_E.5_B2 | AAGGAATAGGAAACCTATTACTAGG |
| SARS-CoV-2_E.5_B1C | TCCAACGGTACTATTACCGTTGA |
| SARS-CoV-2_RNaseP.1_F2 | CCGTGGAGCTTGTTGATGA |
| SARS-CoV-2_RNaseP.1_F1C | GTTGCGGATCCGAGTCAGTGG |
| SARS-CoV-2_RNaseP.1_B2 | TCACGGAGGGGATAAGTGG |
| SARS-CoV-2_RNaseP.1_B1C | AACTCAGCCATCCACATCCGAG |
| SARS-CoV-2_RNaseP.2_F2 | GAGCCAGAGACCGACACA |
| SARS-CoV-2_RNaseP.2_F1C | CGGATGTGGATGGCTGAGTTGT |
| SARS-CoV-2_RNaseP.2_B2 | TGGTCCGAGGTCCAGTAC |
| SARS-CoV-2_RNaseP.2_B1C | ACTCCTCCACTTATCCCCTCCG |
| SARS-CoV-2_RNaseP.3_F2 | CTCCACTTATCCCCTCCGTG |
| SARS-CoV-2_RNaseP.3_F1C | ACATGGCTCTGGTCCGAGGTC |
| SARS-CoV-2_RNaseP.3_B2 | TAACTGGGCCCACCAAGAG |
| SARS-CoV-2_RNaseP.3_B1C | CTGTTCCCTGGAAGCCCAAAGG |
| SARS-CoV-2_RNaseP.4_F2 | GCACACAGCATGGCAGAA |
| SARS-CoV-2_RNaseP.4_F1C | CACTGGATCCAGTTCAGCCTCC |
| SARS-CoV-2_RNaseP.4_B2 | TGGGCCTTAAAGTCCGTCTT |
| SARS-CoV-2_RNaseP.4_B1C | TTAGGAAAAGGCTTCCCAGCCG |
| SARS-CoV-2_RNaseP.5_F2 | AACCGCGCCATCAACATC |
| SARS-CoV-2_RNaseP.5_F1C | CACCTGCAAGGACCCGAAGC |
| SARS-CoV-2_RNaseP.5_B2 | GTTGCGGATCCGAGTCAG |
| SARS-CoV-2_RNaseP.5_B1C | GCCAATACCTCCACCGTGGAG |
| SARS-CoV-2_RegX1.1_F2 | TGACACTAAGAGGGGTGTA |
| SARS-CoV-2_RegX1.1_F1C | TTCCGTGTACCAAGCAATTTCATG |
| SARS-CoV-2_RegX1.1_B2 | TGGACATTCCCCATTGAAG |
| SARS-CoV-2_RegX1.1_B1C | AAGAGCTATGAATTGCAGACACC |
| SARS-CoV-2_RegX1.2_F2 | TGACACTAAGAGGGGTGTA |
| SARS-CoV-2_RegX1.2_F1C | TTCCGTGTACCAAGCAATTTCATG |
| SARS-CoV-2_RegX1.2_B2 | TTGGACATTCCCCATTGA |
| SARS-CoV-2_RegX1.2_B1C | CTGAAAAGAGCTATGAATTGCAGAC |
| SARS-CoV-2_RegX2.1_F2 | TAAGCGTGTTGACTGGAC |
| SARS-CoV-2_RegX2.1_F1C | AGCCGCATTAATCTTCAGTTCATC |
| SARS-CoV-2_RegX2.1_B2 | TAGGGTTACCAATGTCGTGA |
| SARS-CoV-2_RegX2.1_B1C | AGAAAGGTTCAACACATGGTTGT |
| SARS-CoV-2_RegX2.2_F2 | TTAAGCGTGTTGACTGGA |
| SARS-CoV-2_RegX2.2_F1C | GCCGCATTAATCTTCAGTTCATCA |
| SARS-CoV-2_RegX2.2_B2 | TTAGGGTTACCAATGTCGT |
| SARS-CoV-2_RegX2.2_B1C | AGAAAGGTTCAACACATGGTTGTTA |
| SARS-CoV-2_RegX2.3_F2 | TTAAGCGTGTTGACTGGA |
| SARS-CoV-2_RegX2.3_F1C | GCCGCATTAATCTTCAGTTCATCA |
| SARS-CoV-2_RegX2.3_B2 | TTAGGGTTACCAATGTCGT |
| SARS-CoV-2_RegX2.3_B1C | AGAAAGGTTCAACACATGGTTGTT |
| SARS-CoV-2_RegX2.4_F2 | TTAAGCGTGTTGACTGGAC |
| SARS-CoV-2_RegX2.4_F1C | GCCGCATTAATCTTCAGTTCATCA |
| SARS-CoV-2_RegX2.4_B2 | TTAGGGTTACCAATGTCGT |
| SARS-CoV-2_RegX2.4_B1C | AGAAAGGTTCAACACATGGTTGTT |
| SARS-CoV-2_RegX3.1_F2 | AGTTACGTGCCAGATCAG |
| SARS-CoV-2_RegX3.1_F1C | GGAGAGTAAAGTTCTTGAACTTCCT |
| SARS-CoV-2_RegX3.1_B2 | ATGAAAGTTCAATCATTCTGTCT |
| SARS-CoV-2_RegX3.1_B1C | TGCGGCAATAGTGTTTATAACACT |

### Example 19—Primer Design and Tiling

RT-LAMP primers were initially designed using the regions targeted by the CDC SARS-CoV-2 RT-PCR primers and other RT-LAMP primers. Primers were blasted against the target genome using the NCBI's blastn algorithm with the following parameters: word size: 7; expect threshold; 1E11. The regions contained within the resulting alignment of the Forward/Reverse primers (for RT-PCR primers) or F3/B3 primers (for RT-LAMP primers) were exported to FASTA file format. If the region identified by primer alignment was less than 1200 nucleotides, the identified region was padded equally on both sides with nucleotides corresponding to the organism's sequence until the total length of the region was approximately 1200 nucleotides. The RdRP gene was divided into two regions to ensure that the sequences were less than 2,000 nucleotides in length.

Additional regions were identified by separating the SARS-CoV-2 genome (Accession #: NC_045512.2) into portions of 2,000 nucleotides. The regions overlapped by 500 nucleotides. Each of these regions were referred to as Tiled Regions. For example, Tiled region 1 would be the nucleotide sequence from position 0 to position 2000 of the reference genome, tiled region 2 from 1,500 to 3,500, tiled region 3 from 3,000 to 5,000, and so forth.

Each Tiling Region was used as the input into the Primer Explorer v5 algorithm. The algorithm parameters were adjusted to design primers (most notably the length of the primers and distance between primers). Primer sets for targeted regions from the CDC and literature were chosen based on their end stability, namely the 5′ end of the F1c/B1c and the 3′ end of the F3/B3/F2/B2/LF/LB should be less than −4.00 kcal/mol (i.e., more negative). If these restrictions could not be maintained, then the primer sets with closest end stabilities to −4.00 were selected. Selected primer sets were used as inputs to design loop primers in the Primer Explorer v5 algorithm. Loop primers with melting temperatures closest to 65° C. were chosen provided they still maintained the thermodynamic parameters previously described in this disclosure.

Tiled regions were used as input into the Primer Explorer v5 algorithm. Parameters were set to maximize the number of returned primer sets by: (a) reducing the minimum primer dimerization energy, (b) increasing the distance between loop primers and F2, and (c) increasing the maximum number of primer sets returned. Each of the resulting primer sets (which did not include loop primers) was aligned against results from the proprietary FAST-NA algorithm (which determines subsequences with minimal sequence identity to organisms found in the human respiratory tract background, namely human DNA and bacteria/viruses which inhabit the respiratory tract). Primer sets that mostly aligned with these FAST-NA results (less than 5 nucleotides total for all primers outside of the FAST-NA regions) and maintained most of the thermodynamic parameters as previously described were selected for further experimental screening. These primers are indicated by the prefix RegX.

Primer sets selected the preceding were screened experimentally to determine their reaction efficacy and efficiency in order of decreasing priority: (i) number of false positives, (ii) reaction speed, and (iii) limit of detection. Experiments were carried out sequentially in (1) solution (water followed by saliva) using fluorometric RT-LAMP, then (2) colorimetric RT-LAMP in solution, and finally (3) colorimetric RT-LAMP on paper. Screened primer sets were experimentally tested for cross-reactivity against other organisms in the human respiratory tract.

EXAMPLE 20 - SARS-CoV-2_N
SARS-CoV-2 N can have the sequence:
ATGTCTGATAATGGACCCCAAAATCAGCGAAATGCACCCCGCATTACGTTTGGTGGAC
CCTCAGATTCAACTGGCAGTAACCAGAATGGAGAACGCAGTGGGGCGCGATCAAAA
CAACGTCGGCCCCAAGGTTTACCCAATAATACTGCGTCTTGGTTCACCGCTCTCACTC
AACATGGCAAGGAAGACCTTAAATTCCCTCGAGGACAAGGCGTTCCAATTAACACCA
ATAGCAGTCCAGATGACCAAATTGGCTACTACCGAAGAGCTACCAGACGAATTCGTG
GTGGTGACGGTAAAATGAAAGATCTCAGTCCAAGATGGTATTTCTACTACCTAGGAAC
TGGGCCAGAAGCTGGACTTCCCTATGGTGCTAACAAAGACGGCATCATATGGGTTGC
AACTGAGGGAGCCTTGAATACACCAAAAGATCACATTGGCACCCGCAATCCTGCTAA
CAATGCTGCAATCGTGCTACAACTTCCTCAAGGAACAACATTGCCAAAAGGCTTCTA
CGCAGAAGGGAGCAGAGGCGGCAGTCAAGCCTCTTCTCGTTCCTCATCACGTAGTCG
CAACAGTTCAAGAAATTCAACTCCAGGCAGCAGTAGGGGAACTTCTCCTGCTAGAAT
GGCTGGCAATGGCGGTGATGCTGCTCTTGCTTTGCTGCTGCTTGACAGATTGAACCAG
CTTGAGAGCAAAATGTCTGGTAAAGGCCAACAACAACAAGGCCAAACTGTCACTAA
GAAATCTGCTGCTGAGGCTTCTAAGAAGCCTCGGCAAAAACGTACTGCCACTAAAGC
ATACAATGTAACACAAGCTTTCGGCAGACGTGGTCCAGAACAAACCCAAGGAAATTT
TGGGGACCAGGAACTAATCAGACAAGGAACTGATTACAAACATTGGCCGCAAATTGC
ACAATTTGCCCCCAGCGCTTCAGCGTTCTTCGGAATGTCGCGCATTGGCATGGAAGTC
ACACCTTCGGGAACGTGGTTGACCTACACAGGTGCCATCAAATTGGATGACAAAGAT
CCAAATTTCAAAGATCAAGTCATTTTGCTGAATAAGCATATTGACGCATACAAAACATT
CCCACCAACAGAGCCTAAAAAGGACAAAAAGAAGAAGGCTGATGAAACTCAAGCCT
TACCGCAGAGACAGAAGAAACAGCAAACTGTGACTCTTCTTCCTGCTGCAGATTTGG
ATGATTTCTCCAAACAATTGCAACAATCCATGAGCAGTGCTGACTCAACTCAGGCCTA
A.
SARS-CoV-2 N antisense can have the sequence:
TTAGGCCTGAGTTGAGTCAGCACTGCTCATGGATTGTTGCAATTGTTTGGAGAAATCA
TCCAAATCTGCAGCAGGAAGAAGAGTCACAGTTTGCTGTTTCTTCTGTCTCTGCGGTA
AGGCTTGAGTTTCATCAGCCTTCTTCTTTTTGTCCTTTTTAGGCTCTGTTGGTGGGAAT
GTTTTGTATGCGTCAATATGCTTATTCAGCAAAATGACTTGATCTTTGAAATTTGGATCT
TTGTCATCCAATTTGATGGCACCTGTGTAGGTCAACCACGTTCCCGAAGGTGTGACTT
CCATGCCAATGCGCGACATTCCGAAGAACGCTGAAGCGCTGGGGGCAAATTGTGCAA
TTTGCGGCCAATGTTTGTAATCAGTTCCTTGTCTGATTAGTTCCTGGTCCCCAAAATTT
CCTTGGGTTTGTTCTGGACCACGTCTGCCGAAAGCTTGTGTTACATTGTATGCTTTAGT
GGCAGTACGTTTTTGCCGAGGCTTCTTAGAAGCCTCAGCAGCAGATTTCTTAGTGACA
GTTTGGCCTTGTTGTTGTTGGCCTTTACCAGACATTTTGCTCTCAAGCTGGTTCAATCT
GTCAAGCAGCAGCAAAGCAAGAGCAGCATCACCGCCATTGCCAGCCATTCTAGCAGG
AGAAGTTCCCCTACTGCTGCCTGGAGTTGAATTTCTTGAACTGTTGCGACTACGTGAT
GAGGAACGAGAAGAGGCTTGACTGCCGCCTCTGCTCCCTTCTGCGTAGAAGCCTTTT
GGCAATGTTGTTCCTTGAGGAAGTTGTAGCACGATTGCAGCATTGTTAGCAGGATTGC
GGGTGCCAATGTGATCTTTTGGTGTATTCAAGGCTCCCTCAGTTGCAACCCATATGATG
CCGTCTTTGTTAGCACCATAGGGAAGTCCAGCTTCTGGCCCAGTTCCTAGGTAGTAGA
AATACCATCTTGGACTGAGATCTTTCATTTTACCGTCACCACCACGAATTCGTCTGGTA
GCTCTTCGGTAGTAGCCAATTTGGTCATCTGGACTGCTATTGGTGTTAATTGGAACGCC
TTGTCCTCGAGGGAATTTAAGGTCTTCCTTGCCATGTTGAGTGAGAGCGGTGAACCA
AGACGCAGTATTATTGGGTAAACCTTGGGGCCGACGTTGTTTTGATCGCGCCCCACTG
CGTTCTCCATTCTGGTTACTGCCAGTTGAATCTGAGGGTCCACCAAACGTAATGCGGG
GTGCATTTCGCTGATTTTGGGGTCCATTATCAGACAT.
EXAMPLE 21 - SARS-CoV-2 orf1ab
SARS-CoV-2 orf1ab can have the sequence:
AGGGAGGTAGGTTTGTACTTGCACTGTTATCCGATTTACAGGATTTGAAATGGGCTAG
ATTCCCTAAGAGTGATGGAACTGGTACTATCTATACAGAACTGGAACCACCTTGTAGG
TTTGTTACAGACACACCTAAAGGTCCTAAAGTGAAGTATTTATACTTTATTAAAGGATT
AAACAACCTAAATAGAGGTATGGTACTTGGTAGTTTAGCTGCCACAGTACGTCTACAA
GCTGGTAATGCAACAGAAGTGCCTGCCAATTCAACTGTATTATCTTTCTGTGCTTTTGC
TGTAGATGCTGCTAAAGCTTACAAAGATTATCTAGCTAGTGGGGGACAACCAATCACT
AATTGTGTTAAGATGTTGTGTACACACACTGGTACTGGTCAGGCAATAACAGTTACAC
CGGAAGCCAATATGGATCAAGAATCCTTTGGTGGTGCATCGTGTTGTCTGTACTGCCG
TTGCCACATAGATCATCCAAATCCTAAAGGATTTTGTGACTTAAAAGGTAAGTATGTAC
AAATACCTACAACTTGTGCTAATGACCCTGTGGGTTTTACACTTAAAAACACAGTCTG
TACCGTCTGCGGTATGTGGAAAGGTTATGGCTGTAGTTGTGATCAACTCCGCGAACCC
ATGCTTCAGTCAGCTGATGCACAATCGTTTTTAAACGGGTTTGCGGTGTAAGTGCAGC
CCGTCTTACACCGTGCGGCACAGGCACTAGTACTGATGTCGTATACAGGGCTTTTGAC
ATCTACAATGATAAAGTAGCTGGTTTTGCTAAATTCCTAAAAACTAATTGTTGTCGCTT
CCAAGAAAAGGACGAAGATGACAATTTAATTGATTCTTACTTTGTAGTTAAGAGACAC
ACTTTCTCTAACTACCAACATGAAGAAACAATTTATAATTTACTTAAGGATTGTCCAGC
TGTTGCTAAACATGACTTCTTTAAGTTTAGAATAGACGGTGACATGGTACCACATATAT
CACGTCAACGTCTTACTAAATACACAATGGCAGACCTCGTCTATGCTTTAAGGCATTTT
GATGAAGGTAATTGTGACACATTAAAAGAAATACTTGTCACATACAATTGTTGTGATG
ATGATTATTTCAATAAAAAGGACTGGTATGATTTTGTAGAAAACCCAGATATATTACGC
GTATACGCCAACTTAGGTGAACGTGTACGCCAAGCTTTGTTAAAAACAGTA.
SARS-CoV-2 orf1ab antisense can have the sequence:
TACTGTTTTTAACAAAGCTTGGCGTACACGTTCACCTAAGTTGGCGTATACGCGTAATA
TATCTGGGTTTTCTACAAAATCATACCAGTCCTTTTTATTGAAATAATCATCATCACAAC
AATTGTATGTGACAAGTATTTCTTTTAATGTGTCACAATTACCTTCATCAAAATGCCTTA
AAGCATAGACGAGGTCTGCCATTGTGTATTTAGTAAGACGTTGACGTGATATATGTGGT
ACCATGTCACCGTCTATTCTAAACTTAAAGAAGTCATGTTTAGCAACAGCTGGACAAT
CCTTAAGTAAATTATAAATTGTTTCTTCATGTTGGTAGTTAGAGAAAGTGTGTCTCTTA
ACTACAAAGTAAGAATCAATTAAATTGTCATCTTCGTCCTTTTCTTGGAAGCGACAAC
AATTAGTTTTTAGGAATTTAGCAAAACCAGCTACTTTATCATTGTAGATGTCAAAAGCC
CTGTATACGACATCAGTACTAGTGCCTGTGCCGCACGGTGTAAGACGGGCTGCACTTA
CACCGCAAACCCGTTTAAAAACGATTGTGCATCAGCTGACTGAAGCATGGGTTCGCG
GAGTTGATCACAACTACAGCCATAACCTTTCCACATACCGCAGACGGTACAGACTGTG
TTTTTAAGTGTAAAACCCACAGGGTCATTAGCACAAGTTGTAGGTATTTGTACATACTT
ACCTTTTAAGTCACAAAATCCTTTAGGATTTGGATGATCTATGTGGCAACGGCAGTAC
AGACAACACGATGCACCACCAAAGGATTCTTGATCCATATTGGCTTCCGGTGTAACTG
TTATTGCCTGACCAGTACCAGTGTGTGTACACAACATCTTAACACAATTAGTGATTGGT
TGTCCCCCACTAGCTAGATAATCTTTGTAAGCTTTAGCAGCATCTACAGCAAAAGCAC
AGAAAGATAATACAGTTGAATTGGCAGGCACTTCTGTTGCATTACCAGCTTGTAGACG
TACTGTGGCAGCTAAACTACCAAGTACCATACCTCTATTTAGGTTGTTTAATCCTTTAAT
AAAGTATAAATACTTCACTTTAGGACCTTTAGGTGTGTCTGTAACAAACCTACAAGGT
GGTTCCAGTTCTGTATAGATAGTACCAGTTCCATCACTCTTAGGGAATCTAGCCCATTT
CAAATCCTGTAAATCGGATAACAGTGCAAGTACAAACCTACCTCCCT.
EXAMPLE 22 - SARS-CoV-2_RdRP-1
SARS-CoV-2 RdRP-1 can have the sequence:
TCAGCTGATGCACAATCGTTTTTAAACGGGTTTGCGGTGTAAGTGCAGCCCGTCTTAC
ACCGTGCGGCACAGGCACTAGTACTGATGTCGTATACAGGGCTTTTGACATCTACAAT
GATAAAGTAGCTGGTTTTGCTAAATTCCTAAAAACTAATTGTTGTCGCTTCCAAGAAA
AGGACGAAGATGACAATTTAATTGATTCTTACTTTGTAGTTAAGAGACACACTTTCTC
TAACTACCAACATGAAGAAACAATTTATAATTTACTTAAGGATTGTCCAGCTGTTGCTA
AACATGACTTCTTTAAGTTTAGAATAGACGGTGACATGGTACCACATATATCACGTCAA
CGTCTTACTAAATACACAATGGCAGACCTCGTCTATGCTTTAAGGCATTTTGATGAAGG
TAATTGTGACACATTAAAAGAAATACTTGTCACATACAATTGTTGTGATGATGATTATT
TCAATAAAAAGGACTGGTATGATTTTGTAGAAAACCCAGATATATTACGCGTATACGCC
AACTTAGGTGAACGTGTACGCCAAGCTTTGTTAAAAACAGTACAATTCTGTGATGCCA
TGCGAAATGCTGGTATTGTTGGTGTACTGACATTAGATAATCAAGATCTCAATGGTAAC
TGGTATGATTTCGGTGATTTCATACAAACCACGCCAGGTAGTGGAGTTCCTGTTGTAG
ATTCTTATTATTCATTGTTAATGCCTATATTAACCTTGACCAGGGCTTTAACTGCAGAGT
CACATGTTGACACTGACTTAACAAAGCCTTACATTAAGTGGGATTTGTTAAAATATGA
CTTCACGGAAGAGAGGTTAAAACTCTTTGACCGTTATTTTAAATATTGGGATCAGACA
TACCACCCAAATTGTGTTAACTGTTTGGATGACAGATGCATTCTGCATTGTGCAAACT
TTAATGTTTTATTCTCTACAGTGTTCCCACCTACAAGTTTTGGACCACTAGTGAGAAAA
ATATTTGTTGATGGTGTTCCATTTGTAGTTTCAACTGGATACCACTTCAGAGAGCTAGG
TGTTGTACATAATCAGGATGTAAACTTACATAGCTCTAGACTTAGTTTTAAGGAATTAC
TTGTGTATGCTGCTGACCCTGCTATGCACGCTGCTTCTGGTAATCTATTACTAGATAAA
CGCACTACGTGCTTTTCAGTAGCTGCACTTACTAACAATGTTGCTTTTCAAACTGTCA
AACCCGGTAATTTTAACAAAGACTTCTATGACTTTGCTGTGTCTAAGGGTTTCTTTAAG
GAAGGAAGTTCTGTTGAATTAAAACACTTCTTCTTTGCTCAGGATGGTAATGCTGCTA
TCAGCGATTATGACTACTATCGTTATAATCTACCAACAATGTGTGATATCAGACAACTA
CTATTTGTAGTTGAAGTTGTTGATAAGTACTTTGATTGTTACGATGGTGGCTGTATTAAT
GCTAACCAAGTCATCGTCAACAACCTAGACAAATCAGCTGGTTTTCCATTTAATAAAT
GGGGTAAGGCTAGACTTTATTATGATTCAATGAGTTATGAGGATCAAGATGCACTTTTC
GCATATACAAAACGTAATGTCATCCCTACTATAACTCAAATGAATCTTAAGTATGCCATT
AGTGCAAAGAATAGAGCTCGCACCGTAGCTGGTGTCTCTATCTGTAGTACTATGACCA
ATAGACAGTTTCATCAAAAATTATTGAAATCAATAGCCGCCACTAGAGGAGCTACTGT
AGTAATTGGAACAAGCAAATTCTATGGTGGTTGGCACAACATGTTAAAAACTGTTTAT
AGTGATGTAGAAAACCCTCACCTTATGGGTTGGGATTATCCTAAATGTGATAGAGCCAT
GCCTAACATGCTTAGAATTATGGCC.
SARS-CoV-2 RdRP-1 antisense can have the sequence:
GGCCATAATTCTAAGCATGTTAGGCATGGCTCTATCACATTTAGGATAATCCCAACCCA
TAAGGTGAGGGTTTTCTACATCACTATAAACAGTTTTTAACATGTTGTGCCAACCACC
ATAGAATTTGCTTGTTCCAATTACTACAGTAGCTCCTCTAGTGGCGGCTATTGATTTCA
ATAATTTTTGATGAAACTGTCTATTGGTCATAGTACTACAGATAGAGACACCAGCTACG
GTGCGAGCTCTATTCTTTGCACTAATGGCATACTTAAGATTCATTTGAGTTATAGTAGG
GATGACATTACGTTTTGTATATGCGAAAAGTGCATCTTGATCCTCATAACTCATTGAAT
CATAATAAAGTCTAGCCTTACCCCATTTATTAAATGGAAAACCAGCTGATTTGTCTAGG
TTGTTGACGATGACTTGGTTAGCATTAATACAGCCACCATCGTAACAATCAAAGTACTT
ATCAACAACTTCAACTACAAATAGTAGTTGTCTGATATCACACATTGTTGGTAGATTAT
AACGATAGTAGTCATAATCGCTGATAGCAGCATTACCATCCTGAGCAAAGAAGAAGTG
TTTTAATTCAACAGAACTTCCTTCCTTAAAGAAACCCTTAGACACAGCAAAGTCATAG
AAGTCTTTGTTAAAATTACCGGGTTTGACAGTTTGAAAAGCAACATTGTTAGTAAGTG
CAGCTACTGAAAAGCACGTAGTGCGTTTATCTAGTAATAGATTACCAGAAGCAGCGTG
CATAGCAGGGTCAGCAGCATACACAAGTAATTCCTTAAAACTAAGTCTAGAGCTATGT
AAGTTTACATCCTGATTATGTACAACACCTAGCTCTCTGAAGTGGTATCCAGTTGAAA
CTACAAATGGAACACCATCAACAAATATTTTTCTCACTAGTGGTCCAAAACTTGTAGG
TGGGAACACTGTAGAGAATAAAACATTAAAGTTTGCACAATGCAGAATGCATCTGTC
ATCCAAACAGTTAACACAATTTGGGTGGTATGTCTGATCCCAATATTTAAAATAACGGT
CAAAGAGTTTTAACCTCTCTTCCGTGAAGTCATATTTTAACAAATCCCACTTAATGTAA
GGCTTTGTTAAGTCAGTGTCAACATGTGACTCTGCAGTTAAAGCCCTGGTCAAGGTTA
ATATAGGCATTAACAATGAATAATAAGAATCTACAACAGGAACTCCACTACCTGGCGT
GGTTTGTATGAAATCACCGAAATCATACCAGTTACCATTGAGATCTTGATTATCTAATG
TCAGTACACCAACAATACCAGCATTTCGCATGGCATCACAGAATTGTACTGTTTTTAA
CAAAGCTTGGCGTACACGTTCACCTAAGTTGGCGTATACGCGTAATATATCTGGGTTTT
CTACAAAATCATACCAGTCCTTTTTATTGAAATAATCATCATCACAACAATTGTATGTG
ACAAGTATTTCTTTTAATGTGTCACAATTACCTTCATCAAAATGCCTTAAAGCATAGAC
GAGGTCTGCCATTGTGTATTTAGTAAGACGTTGACGTGATATATGTGGTACCATGTCAC
CGTCTATTCTAAACTTAAAGAAGTCATGTTTAGCAACAGCTGGACAATCCTTAAGTAA
ATTATAAATTGTTTCTTCATGTTGGTAGTTAGAGAAAGTGTGTCTCTTAACTACAAAGT
AAGAATCAATTAAATTGTCATCTTCGTCCTTTTCTTGGAAGCGACAACAATTAGTTTTT
AGGAATTTAGCAAAACCAGCTACTTTATCATTGTAGATGTCAAAAGCCCTGTATACGA
CATCAGTACTAGTGCCTGTGCCGCACGGTGTAAGACGGGCTGCACTTACACCGCAAA
CCCGTTTAAAAACGATTGTGCATCAGCTGA.
EXAMPLE 23 - SARS-CoV-2_RdRP-2
SARS-CoV-2 RdRP-2 can have the sequence:
TTAACTGTTTGGATGACAGATGCATTCTGCATTGTGCAAACTTTAATGTTTTATTCTCTA
CAGTGTTCCCACCTACAAGTTTTGGACCACTAGTGAGAAAAATATTTGTTGATGGTGT
TCCATTTGTAGTTTCAACTGGATACCACTTCAGAGAGCTAGGTGTTGTACATAATCAG
GATGTAAACTTACATAGCTCTAGACTTAGTTTTAAGGAATTACTTGTGTATGCTGCTGA
CCCTGCTATGCACGCTGCTTCTGGTAATCTATTACTAGATAAACGCACTACGTGCTTTT
CAGTAGCTGCACTTACTAACAATGTTGCTTTTCAAACTGTCAAACCCGGTAATTTTAA
CAAAGACTTCTATGACTTTGCTGTGTCTAAGGGTTTCTTTAAGGAAGGAAGTTCTGTT
GAATTAAAACACTTCTTCTTTGCTCAGGATGGTAATGCTGCTATCAGCGATTATGACTA
CTATCGTTATAATCTACCAACAATGTGTGATATCAGACAACTACTATTTGTAGTTGAAG
TTGTTGATAAGTACTTTGATTGTTACGATGGTGGCTGTATTAATGCTAACCAAGTCATC
GTCAACAACCTAGACAAATCAGCTGGTTTTCCATTTAATAAATGGGGTAAGGCTAGAC
TTTATTATGATTCAATGAGTTATGAGGATCAAGATGCACTTTTCGCATATACAAAACGT
AATGTCATCCCTACTATAACTCAAATGAATCTTAAGTATGCCATTAGTGCAAAGAATAG
AGCTCGCACCGTAGCTGGTGTCTCTATCTGTAGTACTATGACCAATAGACAGTTTCATC
AAAAATTATTGAAATCAATAGCCGCCACTAGAGGAGCTACTGTAGTAATTGGAACAAG
CAAATTCTATGGTGGTTGGCACAACATGTTAAAAACTGTTTATAGTGATGTAGAAAAC
CCTCACCTTATGGGTTGGGATTATCCTAAATGTGATAGAGCCATGCCTAACATGCTTAG
AATTATGGCCTCACTTGTTCTTGCTCGCAAACATACAACGTGTTGTAGCTTGTCACAC
CGTTTCTATAGATTAGCTAATGAGTGTGCTCAAGTATTGAGTGAAATGGTCATGTGTGG
CGGTTCACTATATGTTAAACCAGGTGGAACCTCATCAGGAGATGCCACAACTGCTTAT
GCTAATAGTGTTTTTAACATTTGTCAAGCTGTCACGGCCAATGTTAATGCACTTTTATC
TACTGATGGTAACAAAATTGCCGATAAGTATGTCCGCAATTTACAACACAGACTTTATG
AGTGTCTCTATAGAAATAGAGATGTTGACACAGACTTTGTGAATGAGTTTTACGCATAT
TTGCGTAAACATTTCTCAATGATGATACTCTCTGACGATGCTGTTGTGTGTTTCAATAG
CACTTATGCATCTCAAGGTCTAGTGGCTAGCATAAAGAACTTTAAGTCAGTTCTTTATT
ATCAAAACAATGTTTTTATGTCTGAAGCAAAATGTTGGACTGAGACTGACCTTACTAA
AGGACCTCATGAATTTTGCTCTCAACATACAATGCTAGTTAAACAGGGTGATGATTATG
TGTACCTTCCTTACCCAGATCCATCAAGAATCCTAGGGGCCGGCTGTTTTGTAGATGAT
ATCGTAAAAACAGATGGTACACTTATGATTGAACGGTTCGTGTCTTTAGCTATAGATGC
TTACCCACTTACTAAACATCCTAATCAGGAGTATGCTGATGTCTTTCATTTGTACTTACA
ATACATAAGAAAGCTACATGATGAGTTAACAGGACACATGTTAGACATGTATTCTGTTA
TGCTTACTAATGATAACACTTCAAGGTATTGGGAACCTGAGTTTTATGAGGCTATGTAC
ACACCGCATACAGTCTTACAG.
SARS-CoV-2 RdRP-2 antisense can have the sequence:
CTGTAAGACTGTATGCGGTGTGTACATAGCCTCATAAAACTCAGGTTCCCAATACCTT
GAAGTGTTATCATTAGTAAGCATAACAGAATACATGTCTAACATGTGTCCTGTTAACTC
ATCATGTAGCTTTCTTATGTATTGTAAGTACAAATGAAAGACATCAGCATACTCCTGAT
TAGGATGTTTAGTAAGTGGGTAAGCATCTATAGCTAAAGACACGAACCGTTCAATCAT
AAGTGTACCATCTGTTTTTACGATATCATCTACAAAACAGCCGGCCCCTAGGATTCTTG
ATGGATCTGGGTAAGGAAGGTACACATAATCATCACCCTGTTTAACTAGCATTGTATGT
TGAGAGCAAAATTCATGAGGTCCTTTAGTAAGGTCAGTCTCAGTCCAACATTTTGCTT
CAGACATAAAAACATTGTTTTGATAATAAAGAACTGACTTAAAGTTCTTTATGCTAGCC
ACTAGACCTTGAGATGCATAAGTGCTATTGAAACACACAACAGCATCGTCAGAGAGT
ATCATCATTGAGAAATGTTTACGCAAATATGCGTAAAACTCATTCACAAAGTCTGTGTC
AACATCTCTATTTCTATAGAGACACTCATAAAGTCTGTGTTGTAAATTGCGGACATACT
TATCGGCAATTTTGTTACCATCAGTAGATAAAAGTGCATTAACATTGGCCGTGACAGCT
TGACAAATGTTAAAAACACTATTAGCATAAGCAGTTGTGGCATCTCCTGATGAGGTTC
CACCTGGTTTAACATATAGTGAACCGCCACACATGACCATTTCACTCAATACTTGAGC
ACACTCATTAGCTAATCTATAGAAACGGTGTGACAAGCTACAACACGTTGTATGTTTG
CGAGCAAGAACAAGTGAGGCCATAATTCTAAGCATGTTAGGCATGGCTCTATCACATT
TAGGATAATCCCAACCCATAAGGTGAGGGTTTTCTACATCACTATAAACAGTTTTTAAC
ATGTTGTGCCAACCACCATAGAATTTGCTTGTTCCAATTACTACAGTAGCTCCTCTAGT
GGCGGCTATTGATTTCAATAATTTTTGATGAAACTGTCTATTGGTCATAGTACTACAGAT
AGAGACACCAGCTACGGTGCGAGCTCTATTCTTTGCACTAATGGCATACTTAAGATTC
ATTTGAGTTATAGTAGGGATGACATTACGTTTTGTATATGCGAAAAGTGCATCTTGATC
CTCATAACTCATTGAATCATAATAAAGTCTAGCCTTACCCCATTTATTAAATGGAAAAC
CAGCTGATTTGTCTAGGTTGTTGACGATGACTTGGTTAGCATTAATACAGCCACCATCG
TAACAATCAAAGTACTTATCAACAACTTCAACTACAAATAGTAGTTGTCTGATATCACA
CATTGTTGGTAGATTATAACGATAGTAGTCATAATCGCTGATAGCAGCATTACCATCCTG
AGCAAAGAAGAAGTGTTTTAATTCAACAGAACTTCCTTCCTTAAAGAAACCCTTAGA
CACAGCAAAGTCATAGAAGTCTTTGTTAAAATTACCGGGTTTGACAGTTTGAAAAGC
AACATTGTTAGTAAGTGCAGCTACTGAAAAGCACGTAGTGCGTTTATCTAGTAATAGA
TTACCAGAAGCAGCGTGCATAGCAGGGTCAGCAGCATACACAAGTAATTCCTTAAAA
CTAAGTCTAGAGCTATGTAAGTTTACATCCTGATTATGTACAACACCTAGCTCTCTGAA
GTGGTATCCAGTTGAAACTACAAATGGAACACCATCAACAAATATTTTTCTCACTAGT
GGTCCAAAACTTGTAGGTGGGAACACTGTAGAGAATAAAACATTAAAGTTTGCACAA
TGCAGAATGCATCTGTCATCCAAACAGTTAA.
EXAMPLE 24 - RNaseP POP7-mRNA
RNaseP POP7-mRNA can have the sequence:
ACTCCGCAGCCCGTTCAGGACCCCGGCGCGGGCAGGGCGCCCACGAGCTGGCTGGC
TGCTTGCACCCACATCCTTCTTTCTCTGGGACCTGGGGTCGCGGTTACTTGGGCTGGC
CGGCGAACCCTTGAGTGGCCTGGCGGGGAGCGGGCCTCGCGCGCCTGGAGGGCCCT
GTGGAACGAAGAGAGGCACACAGCATGGCAGAAAACCGAGAGCCCCGCGGTGCTG
TGGAGGCTGAACTGGATCCAGTGGAATACACCCTTAGGAAAAGGCTTCCCAGCCGCC
TGCCCCGGAGACCCAATGACATTTATGTCAACATGAAGACGGACTTTAAGGCCCAGC
TGGCCCGCTGCCAGAAGCTGCTGGACGGAGGGGCCCGGGGTCAGAACGCGTGCTCT
GAGATCTACATTCACGGCTTGGGCCTGGCCATCAACCGCGCCATCAACATCGCGCTGC
AGCTGCAGGCGGGCAGCTTCGGGTCCTTGCAGGTGGCTGCCAATACCTCCACCGTGG
AGCTTGTTGATGAGCTGGAGCCAGAGACCGACACACGGGAGCCACTGACTCGGATC
CGCAACAACTCAGCCATCCACATCCGAGTCTTCAGGGTCACACCCAAGTAATTGAAA
AGACACTCCTCCACTTATCCCCTCCGTGATATGGCTCTTCGCATGCTGAGTACTGGACC
TCGGACCAGAGCCATGTAAGAAAAGGCCTGTTCCCTGGAAGCCCAAAGGACTCTGC
ATTGAGGGTGGGGGTAATTGTCTCTTGGTGGGCCCAGTTAGTGGGCCTTCCTGAGTGT
GTGTATGCGGTCTGTAACTATTGCCATATAAATAAAAAATCCTGTTGCACTAGT.
RNaseP POP7-mRNA antisense can have the sequence:
ACTAGTGCAACAGGATTTTTTATTTATATGGCAATAGTTACAGACCGCATACACACACT
CAGGAAGGCCCACTAACTGGGCCCACCAAGAGACAATTACCCCCACCCTCAATGCAG
AGTCCTTTGGGCTTCCAGGGAACAGGCCTTTTCTTACATGGCTCTGGTCCGAGGTCCA
GTACTCAGCATGCGAAGAGCCATATCACGGAGGGGATAAGTGGAGGAGTGTCTTTTC
AATTACTTGGGTGTGACCCTGAAGACTCGGATGTGGATGGCTGAGTTGTTGCGGATCC
GAGTCAGTGGCTCCCGTGTGTCGGTCTCTGGCTCCAGCTCATCAACAAGCTCCACGG
TGGAGGTATTGGCAGCCACCTGCAAGGACCCGAAGCTGCCCGCCTGCAGCTGCAGC
GCGATGTTGATGGCGCGGTTGATGGCCAGGCCCAAGCCGTGAATGTAGATCTCAGAG
CACGCGTTCTGACCCCGGGCCCCTCCGTCCAGCAGCTTCTGGCAGCGGGCCAGCTGG
GCCTTAAAGTCCGTCTTCATGTTGACATAAATGTCATTGGGTCTCCGGGGCAGGCGGC
TGGGAAGCCTTTTCCTAAGGGTGTATTCCACTGGATCCAGTTCAGCCTCCACAGCACC
GCGGGGCTCTCGGTTTTCTGCCATGCTGTGTGCCTCTCTTCGTTCCACAGGGCCCTCC
AGGCGCGCGAGGCCCGCTCCCCGCCAGGCCACTCAAGGGTTCGCCGGCCAGCCCAA
GTAACCGCGACCCCAGGTCCCAGAGAAAGAAGGATGTGGGTGCAAGCAGCCAGCCA
GCTCGTGGGCGCCCTGCCCGCGCCGGGGTCCTGAACGGGCTGCGGAGT.
EXAMPLE 25 - RegX1
RegX1 can have the sequence:
ATTAAAGGTTTATACCTTCCCAGGTAACAAACCAACCAACTTTCGATCTCTTGTAGATC
TGTTCTCTAAACGAACTTTAAAATCTGTGTGGCTGTCACTCGGCTGCATGCTTAGTGC
ACTCACGCAGTATAATTAATAACTAATTACTGTCGTTGACAGGACACGAGTAACTCGT
CTATCTTCTGCAGGCTGCTTACGGTTTCGTCCGTGTTGCAGCCGATCATCAGCACATCT
AGGTTTCGTCCGGGTGTGACCGAAAGGTAAGATGGAGAGCCTTGTCCCTGGTTTCAA
CGAGAAAACACACGTCCAACTCAGTTTGCCTGTTTTACAGGTTCGCGACGTGCTCGT
ACGTGGCTTTGGAGACTCCGTGGAGGAGGTCTTATCAGAGGCACGTCAACATCTTAA
AGATGGCACTTGTGGCTTAGTAGAAGTTGAAAAAGGCGTTTTGCCTCAACTTGAACA
GCCCTATGTGTTCATCAAACGTTCGGATGCTCGAACTGCACCTCATGGTCATGTTATGG
TTGAGCTGGTAGCAGAACTCGAAGGCATTCAGTACGGTCGTAGTGGTGAGACACTTG
GTGTCCTTGTCCCTCATGTGGGCGAAATACCAGTGGCTTACCGCAAGGTTCTTCTTCG
TAAGAACGGTAATAAAGGAGCTGGTGGCCATAGTTACGGCGCCGATCTAAAGTCATTT
GACTTAGGCGACGAGCTTGGCACTGATCCTTATGAAGATTTTCAAGAAAACTGGAAC
ACTAAACATAGCAGTGGTGTTACCCGTGAACTCATGCGTGAGCTTAACGGAGGGGCA
TACACTCGCTATGTCGATAACAACTTCTGTGGCCCTGATGGCTACCCTCTTGAGTGCAT
TAAAGACCTTCTAGCACGTGCTGGTAAAGCTTCATGCACTTTGTCCGAACAACTGGA
CTTTATTGACACTAAGAGGGGTGTATACTGCTGCCGTGAACATGAGCATGAAATTGCT
TGGTACACGGAACGTTCTGAAAAGAGCTATGAATTGCAGACACCTTTTGAAATTAAAT
TGGCAAAGAAATTTGACACCTTCAATGGGGAATGTCCAAATTTTGTATTTCCCTTAAA
TTCCATAATCAAGACTATTCAACCAAGGGTTGAAAAGAAAAAGCTTGATGGCTTTATG
GGTAGAATTCGATCTGTCTATCCAGTTGCGTCACCAAATGAATGCAACCAAATGTGCC
TTTCAACTCTCATGAAGTGTGATCATTGTGGTGAAACTTCATGGCAGACGGGCGATTT
TGTTAAAGCCACTTGCGAATTTTGTGGCACTGAGAATTTGACTAAAGAAGGTGCCAC
TACTTGTGGTTACTTACCCCAAAATGCTGTTGTTAAAATTTATTGTCCAGCATGTCACA
ATTCAGAAGTAGGACCTGAGCATAGTCTTGCCGAATACCATAATGAATCTGGCTTGAA
AACCATTCTTCGTAAGGGTGGTCGCACTATTGCCTTTGGAGGCTGTGTGTTCTCTTATG
TTGGTTGCCATAACAAGTGTGCCTATTGGGTTCCACGTGCTAGCGCTAACATAGGTTG
TAACCATACAGGTGTTGTTGGAGAAGGTTCCGAAGGTCTTAATGACAACCTTCTTGAA
ATACTCCAAAAAGAGAAAGTCAACATCAATATTGTTGGTGACTTTAAACTTAATGAAG
AGATCGCCATTATTTTGGCATCTTTTTCTGCTTCCACAAGTGCTTTTGTGGAAACTGTG
AAAGGTTTGGATTATAAAGCATTCAAACAAATTGTTGAATCCTGTGGTAATTTTAAAG
TTACAAAAGGAAAAGCTAAAAAAGGTGCCTGGAATATTGGTGAACAGAAATCAATAC
TGAGTCCTCTTTATGCATTTGCATCAGAGGCTGCTCGTGTTGTACGATCAATTTTCTCC
CGCACTCTTGAAACTGCTCAAAATTCTGTGCGTGTTTTACAGAAGGCCGCTATAACAA
TACTAGATGGAATTTCACAGTATTCACTGA.
RegX1 antisense can have the sequence:
TCAGTGAATACTGTGAAATTCCATCTAGTATTGTTATAGCGGCCTTCTGTAAAACACGC
ACAGAATTTTGAGCAGTTTCAAGAGTGCGGGAGAAAATTGATCGTACAACACGAGCA
GCCTCTGATGCAAATGCATAAAGAGGACTCAGTATTGATTTCTGTTCACCAATATTCCA
GGCACCTTTTTTAGCTTTTCCTTTTGTAACTTTAAAATTACCACAGGATTCAACAATTT
GTTTGAATGCTTTATAATCCAAACCTTTCACAGTTTCCACAAAAGCACTTGTGGAAGC
AGAAAAAGATGCCAAAATAATGGCGATCTCTTCATTAAGTTTAAAGTCACCAACAATA
TTGATGTTGACTTTCTCTTTTTGGAGTATTTCAAGAAGGTTGTCATTAAGACCTTCGGA
ACCTTCTCCAACAACACCTGTATGGTTACAACCTATGTTAGCGCTAGCACGTGGAACC
CAATAGGCACACTTGTTATGGCAACCAACATAAGAGAACACACAGCCTCCAAAGGCA
ATAGTGCGACCACCCTTACGAAGAATGGTTTTCAAGCCAGATTCATTATGGTATTCGG
CAAGACTATGCTCAGGTCCTACTTCTGAATTGTGACATGCTGGACAATAAATTTTAAC
AACAGCATTTTGGGGTAAGTAACCACAAGTAGTGGCACCTTCTTTAGTCAAATTCTCA
GTGCCACAAAATTCGCAAGTGGCTTTAACAAAATCGCCCGTCTGCCATGAAGTTTCA
CCACAATGATCACACTTCATGAGAGTTGAAAGGCACATTTGGTTGCATTCATTTGGTG
ACGCAACTGGATAGACAGATCGAATTCTACCCATAAAGCCATCAAGCTTTTTCTTTTC
AACCCTTGGTTGAATAGTCTTGATTATGGAATTTAAGGGAAATACAAAATTTGGACATT
CCCCATTGAAGGTGTCAAATTTCTTTGCCAATTTAATTTCAAAAGGTGTCTGCAATTCA
TAGCTCTTTTCAGAACGTTCCGTGTACCAAGCAATTTCATGCTCATGTTCACGGCAGC
AGTATACACCCCTCTTAGTGTCAATAAAGTCCAGTTGTTCGGACAAAGTGCATGAAGC
TTTACCAGCACGTGCTAGAAGGTCTTTAATGCACTCAAGAGGGTAGCCATCAGGGCC
ACAGAAGTTGTTATCGACATAGCGAGTGTATGCCCCTCCGTTAAGCTCACGCATGAGT
TCACGGGTAACACCACTGCTATGTTTAGTGTTCCAGTTTTCTTGAAAATCTTCATAAGG
ATCAGTGCCAAGCTCGTCGCCTAAGTCAAATGACTTTAGATCGGCGCCGTAACTATGG
CCACCAGCTCCTTTATTACCGTTCTTACGAAGAAGAACCTTGCGGTAAGCCACTGGTA
TTTCGCCCACATGAGGGACAAGGACACCAAGTGTCTCACCACTACGACCGTACTGAA
TGCCTTCGAGTTCTGCTACCAGCTCAACCATAACATGACCATGAGGTGCAGTTCGAGC
ATCCGAACGTTTGATGAACACATAGGGCTGTTCAAGTTGAGGCAAAACGCCTTTTTC
AACTTCTACTAAGCCACAAGTGCCATCTTTAAGATGTTGACGTGCCTCTGATAAGACC
TCCTCCACGGAGTCTCCAAAGCCACGTACGAGCACGTCGCGAACCTGTAAAACAGG
CAAACTGAGTTGGACGTGTGTTTTCTCGTTGAAACCAGGGACAAGGCTCTCCATCTT
ACCTTTCGGTCACACCCGGACGAAACCTAGATGTGCTGATGATCGGCTGCAACACGG
ACGAAACCGTAAGCAGCCTGCAGAAGATAGACGAGTTACTCGTGTCCTGTCAACGAC
AGTAATTAGTTATTAATTATACTGCGTGAGTGCACTAAGCATGCAGCCGAGTGACAGC
CACACAGATTTTAAAGTTCGTTTAGAGAACAGATCTACAAGAGATCGAAAGTTGGTT
GGTTTGTTACCTGGGAAGGTATAAACCTTTAAT.
EXAMPLE 26 - RegX2
RegX2 can have the sequence:
AGTCTTGAAATTCCACGTAGGAATGTGGCAACTTTACAAGCTGAAAATGTAACAGGA
CTCTTTAAAGATTGTAGTAAGGTAATCACTGGGTTACATCCTACACAGGCACCTACAC
ACCTCAGTGTTGACACTAAATTCAAAACTGAAGGTTTATGTGTTGACATACCTGGCAT
ACCTAAGGACATGACCTATAGAAGACTCATCTCTATGATGGGTTTTAAAATGAATTATC
AAGTTAATGGTTACCCTAACATGTTTATCACCCGCGAAGAAGCTATAAGACATGTACG
TGCATGGATTGGCTTCGATGTCGAGGGGTGTCATGCTACTAGAGAAGCTGTTGGTACC
AATTTACCTTTACAGCTAGGTTTTTCTACAGGTGTTAACCTAGTTGCTGTACCTACAGG
TTATGTTGATACACCTAATAATACAGATTTTTCCAGAGTTAGTGCTAAACCACCGCCTG
GAGATCAATTTAAACACCTCATACCACTTATGTACAAAGGACTTCCTTGGAATGTAGT
GCGTATAAAGATTGTACAAATGTTAAGTGACACACTTAAAAATCTCTCTGACAGAGTC
GTATTTGTCTTATGGGCACATGGCTTTGAGTTGACATCTATGAAGTATTTTGTGAAAAT
AGGACCTGAGCGCACCTGTTGTCTATGTGATAGACGTGCCACATGCTTTTCCACTGCT
TCAGACACTTATGCCTGTTGGCATCATTCTATTGGATTTGATTACGTCTATAATCCGTTT
ATGATTGATGTTCAACAATGGGGTTTTACAGGTAACCTACAAAGCAACCATGATCTGT
ATTGTCAAGTCCATGGTAATGCACATGTAGCTAGTTGTGATGCAATCATGACTAGGTGT
CTAGCTGTCCACGAGTGCTTTGTTAAGCGTGTTGACTGGACTATTGAATATCCTATAAT
TGGTGATGAACTGAAGATTAATGCGGCTTGTAGAAAGGTTCAACACATGGTTGTTAAA
GCTGCATTATTAGCAGACAAATTCCCAGTTCTTCACGACATTGGTAACCCTAAAGCTAT
TAAGTGTGTACCTCAAGCTGATGTAGAATGGAAGTTCTATGATGCACAGCCTTGTAGT
GACAAAGCTTATAAAATAGAAGAATTATTCTATTCTTATGCCACACATTCTGACAAATT
CACAGATGGTGTATGCCTATTTTGGAATTGCAATGTCGATAGATATCCTGCTAATTCCAT
TGTTTGTAGATTTGACACTAGAGTGCTATCTAACCTTAACTTGCCTGGTTGTGATGGTG
GCAGTTTGTATGTAAATAAACATGCATTCCACACACCAGCTTTTGATAAAAGTGCTTTT
GTTAATTTAAAACAATTACCATTTTTCTATTACTCTGACAGTCCATGTGAGTCTCATGG
AAAACAAGTAGTGTCAGATATAGATTATGTACCACTAAAGTCTGCTACGTGTATAACAC
GTTGCAATTTAGGTGGTGCTGTCTGTAGACATCATGCTAATGAGTACAGATTGTATCTC
GATGCTTATAACATGATGATCTCAGCTGGCTTTAGCTTGTGGGTTTACAAACAATTTGA
TACTTATAACCTCTGGAACACTTTTACAAGACTTCAGAGTTTAGAAAATGTGGCTTTT
AATGTTGTAAATAAGGGACACTTTGATGGACAACAGGGTGAAGTACCAGTTTCTATCA
TTAATAACACTGTTTACACAAAAGTTGATGGTGTTGATGTAGAATTGTTTGAAAATAA
AACAACATTACCTGTTAATGTAGCATTTGAGCTTTGGGCTAAGCGCAACATTAAACCA
GTACCAGAGGTGAAAATACTCAATAATTTGGGTGTGGACATTGCTGCTAATACTGTGA
TCTGGGACTACAAAAGAGATGCTCCAGCACATATATCTACTATTGGTGTTTGTTCTATG
ACTGACATAGCCAAGAAACCAACTGAAACGATTTGTGCACCACTCACTGTCTTTTTTG
ATGGTAGAGT.
RegX2 antisense can have the sequence:
ACTCTACCATCAAAAAAGACAGTGAGTGGTGCACAAATCGTTTCAGTTGGTTTCTTG
GCTATGTCAGTCATAGAACAAACACCAATAGTAGATATATGTGCTGGAGCATCTCTTTT
GTAGTCCCAGATCACAGTATTAGCAGCAATGTCCACACCCAAATTATTGAGTATTTTCA
CCTCTGGTACTGGTTTAATGTTGCGCTTAGCCCAAAGCTCAAATGCTACATTAACAGG
TAATGTTGTTTTATTTTCAAACAATTCTACATCAACACCATCAACTTTTGTGTAAACAG
TGTTATTAATGATAGAAACTGGTACTTCACCCTGTTGTCCATCAAAGTGTCCCTTATTT
ACAACATTAAAAGCCACATTTTCTAAACTCTGAAGTCTTGTAAAAGTGTTCCAGAGGT
TATAAGTATCAAATTGTTTGTAAACCCACAAGCTAAAGCCAGCTGAGATCATCATGTTA
TAAGCATCGAGATACAATCTGTACTCATTAGCATGATGTCTACAGACAGCACCACCTA
AATTGCAACGTGTTATACACGTAGCAGACTTTAGTGGTACATAATCTATATCTGACACT
ACTTGTTTTCCATGAGACTCACATGGACTGTCAGAGTAATAGAAAAATGGTAATTGTT
TTAAATTAACAAAAGCACTTTTATCAAAAGCTGGTGTGTGGAATGCATGTTTATTTACA
TACAAACTGCCACCATCACAACCAGGCAAGTTAAGGTTAGATAGCACTCTAGTGTCA
AATCTACAAACAATGGAATTAGCAGGATATCTATCGACATTGCAATTCCAAAATAGGC
ATACACCATCTGTGAATTTGTCAGAATGTGTGGCATAAGAATAGAATAATTCTTCTATT
TTATAAGCTTTGTCACTACAAGGCTGTGCATCATAGAACTTCCATTCTACATCAGCTTG
AGGTACACACTTAATAGCTTTAGGGTTACCAATGTCGTGAAGAACTGGGAATTTGTCT
GCTAATAATGCAGCTTTAACAACCATGTGTTGAACCTTTCTACAAGCCGCATTAATCTT
CAGTTCATCACCAATTATAGGATATTCAATAGTCCAGTCAACACGCTTAACAAAGCAC
TCGTGGACAGCTAGACACCTAGTCATGATTGCATCACAACTAGCTACATGTGCATTAC
CATGGACTTGACAATACAGATCATGGTTGCTTTGTAGGTTACCTGTAAAACCCCATTGT
TGAACATCAATCATAAACGGATTATAGACGTAATCAAATCCAATAGAATGATGCCAAC
AGGCATAAGTGTCTGAAGCAGTGGAAAAGCATGTGGCACGTCTATCACATAGACAAC
AGGTGCGCTCAGGTCCTATTTTCACAAAATACTTCATAGATGTCAACTCAAAGCCATG
TGCCCATAAGACAAATACGACTCTGTCAGAGAGATTTTTAAGTGTGTCACTTAACATT
TGTACAATCTTTATACGCACTACATTCCAAGGAAGTCCTTTGTACATAAGTGGTATGAG
GTGTTTAAATTGATCTCCAGGCGGTGGTTTAGCACTAACTCTGGAAAAATCTGTATTAT
TAGGTGTATCAACATAACCTGTAGGTACAGCAACTAGGTTAACACCTGTAGAAAAACC
TAGCTGTAAAGGTAAATTGGTACCAACAGCTTCTCTAGTAGCATGACACCCCTCGACA
TCGAAGCCAATCCATGCACGTACATGTCTTATAGCTTCTTCGCGGGTGATAAACATGTT
AGGGTAACCATTAACTTGATAATTCATTTTAAAACCCATCATAGAGATGAGTCTTCTAT
AGGTCATGTCCTTAGGTATGCCAGGTATGTCAACACATAAACCTTCAGTTTTGAATTTA
GTGTCAACACTGAGGTGTGTAGGTGCCTGTGTAGGATGTAACCCAGTGATTACCTTAC
TACAATCTTTAAAGAGTCCTGTTACATTTTCAGCTTGTAAAGTTGCCACATTCCTACGT
GGAATTTCAAGACT.
EXAMPLE 27 - RegX3
RegX3 can have the sequence:
ACATCAAGGACCTGCCTAAAGAAATCACTGTTGCTACATCACGAACGCTTTCTTATTA
CAAATTGGGAGCTTCGCAGCGTGTAGCAGGTGACTCAGGTTTTGCTGCATACAGTCG
CTACAGGATTGGCAACTATAAATTAAACACAGACCATTCCAGTAGCAGTGACAATATT
GCTTTGCTTGTACAGTAAGTGACAACAGATGTTTCATCTCGTTGACTTTCAGGTTACT
ATAGCAGAGATATTACTAATTATTATGAGGACTTTTAAAGTTTCCATTTGGAATCTTGAT
TACATCATAAACCTCATAATTAAAAATTTATCTAAGTCACTAACTGAGAATAAATATTCT
CAATTAGATGAAGAGCAACCAATGGAGATTGATTAAACGAACATGAAAATTATTCTTT
TCTTGGCACTGATAACACTCGCTACTTGTGAGCTTTATCACTACCAAGAGTGTGTTAG
AGGTACAACAGTACTTTTAAAAGAACCTTGCTCTTCTGGAACATACGAGGGCAATTC
ACCATTTCATCCTCTAGCTGATAACAAATTTGCACTGACTTGCTTTAGCACTCAATTTG
CTTTTGCTTGTCCTGACGGCGTAAAACACGTCTATCAGTTACGTGCCAGATCAGTTTC
ACCTAAACTGTTCATCAGACAAGAGGAAGTTCAAGAACTTTACTCTCCAATTTTTCTT
ATTGTTGCGGCAATAGTGTTTATAACACTTTGCTTCACACTCAAAAGAAAGACAGAAT
GATTGAACTTTCATTAATTGACTTCTATTTGTGCTTTTTAGCCTTTCTGCTATTCCTTGT
TTTAATTATGCTTATTATCTTTTGGTTCTCACTTGAACTGCAAGATCATAATGAAACTTG
TCACGCCTAAACGAACATGAAATTTCTTGTTTTCTTAGGAATCATCACAACTGTAGCT
GCATTTCACCAAGAATGTAGTTTACAGTCATGTACTCAACATCAACCATATGTAGTTGA
TGACCCGTGTCCTATTCACTTCTATTCTAAATGGTATATTAGAGTAGGAGCTAGAAAAT
CAGCACCTTTAATTGAATTGTGCGTGGATGAGGCTGGTTCTAAATCACCCATTCAGTA
CATCGATATCGGTAATTATACAGTTTCCTGTTTACCTTTTACAATTAATTGCCAGGAACC
TAAATTGGGTAGTCTTGTAGTGCGTTGTTCGTTCTATGAAGACTTTTTAGAGTATCATG
ACGTTCGTGTTGTTTTAGATTTCATCTAAACGAACAAACTAAAATGTCTGATAATGGA
CCCCAAAATCAGCGAAATGCACCCCGCATTACGTTTGGTGGACCCTCAGATTCAACTG
GCAGTAACCAGAATGGAGAACGCAGTGGGGCGCGATCAAAACAACGTCGGCCCCAA
GGTTTACCCAATAATACTGCGTCTTGGTTCACCGCTCTCACTCAACATGGCAAGGAAG
ACCTTAAATTCCCTCGAGGACAAGGCGTTCCAATTAACACCAATAGCAGTCCAGATGA
CCAAATTGGCTACTACCGAAGAGCTACCAGACGAATTCGTGGTGGTGACGGTAAAAT
GAAAGATCTCAGTCCAAGATGGTATTTCTACTACCTAGGAACTGGGCCAGAAGCTGG
ACTTCCCTATGGTGCTAACAAAGACGGCATCATATGGGTTGCAACTGAGGGAGCCTTG
AATACACCAAAAGATCACATTGGCACCCGCAATCCTGCTAACAATGCTGCAATCGTGC
TACAACTTCCTCAAGGAACAACATTGCCAAAAGGCTTCTACGCAGAAGGGAGCAGA
GGCGGCAGTCAAGCCTCTTCTCGTTCCTCATCACGTAGTCGCAACAGTTCAAGAAATT
CAACTCCAGGCAGCAGTAGGGGAACTTCTCCTGCTAGAATGGCTGGCAATGGCGGTG
ATGCTGCTCTTGCTTTGCTGCTGCTTGACAGATTGAACCAGCTTGAGAGCAAAATGTC
TGGTAAAGGCCAACAACAACAAG.
RegX3 antisense can have the sequence:
CTTGTTGTTGTTGGCCTTTACCAGACATTTTGCTCTCAAGCTGGTTCAATCTGTCAAGC
AGCAGCAAAGCAAGAGCAGCATCACCGCCATTGCCAGCCATTCTAGCAGGAGAAGTT
CCCCTACTGCTGCCTGGAGTTGAATTTCTTGAACTGTTGCGACTACGTGATGAGGAAC
GAGAAGAGGCTTGACTGCCGCCTCTGCTCCCTTCTGCGTAGAAGCCTTTTGGCAATG
TTGTTCCTTGAGGAAGTTGTAGCACGATTGCAGCATTGTTAGCAGGATTGCGGGTGCC
AATGTGATCTTTTGGTGTATTCAAGGCTCCCTCAGTTGCAACCCATATGATGCCGTCTT
TGTTAGCACCATAGGGAAGTCCAGCTTCTGGCCCAGTTCCTAGGTAGTAGAAATACCA
TCTTGGACTGAGATCTTTCATTTTACCGTCACCACCACGAATTCGTCTGGTAGCTCTTC
GGTAGTAGCCAATTTGGTCATCTGGACTGCTATTGGTGTTAATTGGAACGCCTTGTCCT
CGAGGGAATTTAAGGTCTTCCTTGCCATGTTGAGTGAGAGCGGTGAACCAAGACGCA
GTATTATTGGGTAAACCTTGGGGCCGACGTTGTTTTGATCGCGCCCCACTGCGTTCTC
CATTCTGGTTACTGCCAGTTGAATCTGAGGGTCCACCAAACGTAATGCGGGGTGCATT
TCGCTGATTTTGGGGTCCATTATCAGACATTTTAGTTTGTTCGTTTAGATGAAATCTAA
AACAACACGAACGTCATGATACTCTAAAAAGTCTTCATAGAACGAACAACGCACTAC
AAGACTACCCAATTTAGGTTCCTGGCAATTAATTGTAAAAGGTAAACAGGAAACTGTA
TAATTACCGATATCGATGTACTGAATGGGTGATTTAGAACCAGCCTCATCCACGCACAA
TTCAATTAAAGGTGCTGATTTTCTAGCTCCTACTCTAATATACCATTTAGAATAGAAGT
GAATAGGACACGGGTCATCAACTACATATGGTTGATGTTGAGTACATGACTGTAAACT
ACATTCTTGGTGAAATGCAGCTACAGTTGTGATGATTCCTAAGAAAACAAGAAATTTC
ATGTTCGTTTAGGCGTGACAAGTTTCATTATGATCTTGCAGTTCAAGTGAGAACCAAA
AGATAATAAGCATAATTAAAACAAGGAATAGCAGAAAGGCTAAAAAGCACAAATAGA
AGTCAATTAATGAAAGTTCAATCATTCTGTCTTTCTTTTGAGTGTGAAGCAAAGTGTT
ATAAACACTATTGCCGCAACAATAAGAAAAATTGGAGAGTAAAGTTCTTGAACTTCCT
CTTGTCTGATGAACAGTTTAGGTGAAACTGATCTGGCACGTAACTGATAGACGTGTTT
TACGCCGTCAGGACAAGCAAAAGCAAATTGAGTGCTAAAGCAAGTCAGTGCAAATTT
GTTATCAGCTAGAGGATGAAATGGTGAATTGCCCTCGTATGTTCCAGAAGAGCAAGGT
TCTTTTAAAAGTACTGTTGTACCTCTAACACACTCTTGGTAGTGATAAAGCTCACAAG
TAGCGAGTGTTATCAGTGCCAAGAAAAGAATAATTTTCATGTTCGTTTAATCAATCTCC
ATTGGTTGCTCTTCATCTAATTGAGAATATTTATTCTCAGTTAGTGACTTAGATAAATTT
TTAATTATGAGGTTTATGATGTAATCAAGATTCCAAATGGAAACTTTAAAAGTCCTCAT
AATAATTAGTAATATCTCTGCTATAGTAACCTGAAAGTCAACGAGATGAAACATCTGTT
GTCACTTACTGTACAAGCAAAGCAATATTGTCACTGCTACTGGAATGGTCTGTGTTTA
ATTTATAGTTGCCAATCCTGTAGCGACTGTATGCAGCAAAACCTGAGTCACCTGCTAC
ACGCTGCGAAGCTCCCAATTTGTAATAAGAAAGCGTTCGTGATGTAGCAACAGTGATT
TCTTTAGGCAGGTCCTTGATGT.

### Specific Example Embodiments

In one example, an isolated complementary DNA (cDNA) of a nucleic acid molecule is provided and can comprise: a nucleotide sequence that is at least 85% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 or a combination thereof.

In one example of an isolated complementary DNA (cDNA) of a nucleic acid molecule, the nucleotide sequence can be at least 85% identical to SEQ ID NO: 9.

In another example of an isolated complementary DNA (cDNA) of a nucleic acid molecule, the nucleotide sequence can comprise SEQ ID NO: 1 joined to SEQ ID NO: 2 by a linking sequence selected from Table 11.

In another example of an isolated complementary DNA (cDNA) of a nucleic acid molecule, the nucleotide sequence can be at least 85% identical to SEQ ID NO: 10.

In another example of an isolated complementary DNA (cDNA) of a nucleic acid molecule, the nucleotide sequence can comprise SEQ ID NO: 3 joined to SEQ ID NO: 4 by a linking sequence selected from Table 11.

In another example of an isolated complementary DNA (cDNA) of a nucleic acid molecule, the guanine and cytosine (GC) content of the nucleotide sequence can be 50% or less.

In another example of an isolated complementary DNA (cDNA) of a nucleic acid molecule, the guanine and cytosine (GC) content of the nucleotide sequence can be 40% or less.

In another example of an isolated complementary DNA (cDNA) of a nucleic acid molecule, an end stability of the nucleotide sequence can be less than −3.5 kcal/mol.

In another example of an isolated complementary DNA (cDNA) of a nucleic acid molecule, the nucleotide sequence can have a melting temperature of from about 40° C. to about 62° C.

In another example of an isolated complementary DNA (cDNA) of a nucleic acid molecule, the nucleotide sequence can have a minimum primer dimerization energy of less than −3 kcal/mol.

In another example of an isolated complementary DNA (cDNA) of a nucleic acid molecule, the nucleotide sequence can be less than 50% identical to nucleotide sequences associated with non-target agents.

In another example of an isolated complementary DNA (cDNA) of a nucleic acid molecule, the nucleotide sequence can be at least 90% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 or a combination thereof.

In another example of an isolated complementary DNA (cDNA) of a nucleic acid molecule, the nucleotide sequence can be at least 95% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 or a combination thereof.

In another example of an isolated complementary DNA (cDNA) of a nucleic acid molecule, the nucleotide sequence can be 100% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 or a combination thereof

In one example there is provided a primer set for reverse transcription loop-mediated isothermal amplification (RT-LAMP) analysis that can comprise: a forward inner primer (FIP) sequence that is at least 85% identical to a combination of SEQ ID NO: 1 and SEQ ID NO: 2; a backward inner primer (BIP) sequence that is at least 85% identical to a combination of seq ID NO: 3 and SEQ ID NO: 4. a forward outer primer (F3) sequence that is at least 85% identical to SEQ ID NO: 5; a backward outer primer (B3) sequence that is at least 85% identical to SEQ ID NO: 6; a forward loop primer (LF) sequence that is at least 85% identical to SEQ ID NO: 7; and a backward loop primer (LB) sequence that is at least 85% identical to SEQ ID NO: 8.

In one example of a primer set for reverse transcription loop-mediated isothermal amplification (RT-LAMP) analysis, the FIP sequence can further comprise a linking sequence joining SEQ ID NO: 1 and SEQ ID NO: 2, wherein the linking sequence is selected from Table 11.

In another example of a primer set for reverse transcription loop-mediated isothermal amplification (RT-LAMP) analysis, the BIP sequence can further comprise a linking sequence joining SEQ ID NO: 3 and SEQ ID NO: 4, wherein the linking sequence is selected from Table 11.

In another example of a primer set for reverse transcription loop-mediated isothermal amplification (RT-LAMP) analysis, the guanine and cytosine (GC) content of the FIP, the BIP, the F3, the B3, the LF, the LB, or a combination thereof can be 50% or less.

In another example of a primer set for reverse transcription loop-mediated isothermal amplification (RT-LAMP) analysis, the guanine and cytosine (GC) content of the FIP, the BIP, the F3, the B3, the LF, the LB, or a combination thereof can be 40% or less.

In another example of a primer set for reverse transcription loop-mediated isothermal amplification (RT-LAMP) analysis, an end stability of the FIP, the BIP, the F3, the B3, the LF, the LB, or a combination thereof can be less than −2.5 kcal/mol.

In another example of a primer set for reverse transcription loop-mediated isothermal amplification (RT-LAMP) analysis, the FIP, the BIP, the F3, the B3, the LF, the LB, or a combination thereof can have a melting temperature of from about 40° C. to about 62° C.

In another example of a primer set for reverse transcription loop-mediated isothermal amplification (RT-LAMP) analysis, the FIP, the BIP, the F3, the B3, the LF, the LB, or a combination thereof can have a minimum primer dimerization energy of less than −3.0 kcal/mol.

In another example of a primer set for reverse transcription loop-mediated isothermal amplification (RT-LAMP) analysis, the FIP, the BIP, the F3, the B3, the LF, the LB, or a combination thereof can be less than 50% identical to nucleotide sequences associated with non-target agents.

In another example of a primer set for reverse transcription loop-mediated isothermal amplification (RT-LAMP) analysis, the FIP sequence can be at least 90% identical to a combination of SEQ ID NO: 1 and SEQ ID NO: 2; the BIP sequence can be at least 90% identical to a combination of seq ID NO: 3 and SEQ ID NO: 4; the F3 sequence can be at least 90% identical to SEQ ID NO: 5; the B3 sequence can be at least 90% identical to SEQ ID NO: 6; the LF sequence can be at least 90% identical to SEQ ID NO: 7; and the LB sequence can be at least 90% identical to SEQ ID NO: 8.

In another example of a primer set for reverse transcription loop-mediated isothermal amplification (RT-LAMP) analysis, the FIP sequence can be at least 95% identical to a combination of SEQ ID NO: 1 and SEQ ID NO: 2; the BIP sequence can be at least 95% identical to a combination of seq ID NO: 3 and SEQ ID NO: 4; the F3 sequence can be at least 95% identical to SEQ ID NO: 5; the B3 sequence can be at least 95% identical to SEQ ID NO: 6; the LF sequence can be at least 95% identical to SEQ ID NO: 7; and the LB sequence can be at least 95% identical to SEQ ID NO: 8.

In another example of a primer set for reverse transcription loop-mediated isothermal amplification (RT-LAMP) analysis, the FIP sequence can be at least 100% identical to a combination of SEQ ID NO: 1 and SEQ ID NO: 2, which is equivalent to SEQ ID NO: 9; the BIP sequence can be at least 100% identical to a combination of seq ID NO: 3 and SEQ ID NO: 4, which is equivalent to SEQ ID NO: 10; the F3 sequence is at least 100% identical to SEQ ID NO: 5; the B3 sequence can be at least 100% identical to SEQ ID NO: 6; the LF sequence can be at least 100% identical to SEQ ID NO: 7; and the LB sequence can be at least 100% identical to SEQ ID NO: 8.

In one example there is provided, a method of detecting a target pathogen from a Coronaviridae family in a sample comprising: providing a primer set comprising: a forward inner primer (FIP) sequence that is at least 85% identical to a combination of SEQ ID NO: 1 and SEQ ID NO: 2; a backward inner primer (BIP) sequence that is at least 85% identical to a combination of seq ID NO: 3 and SEQ ID NO: 4. a forward outer primer (F3) sequence that is at least 85% identical to SEQ ID NO: 5; a backward outer primer (B3) sequence that is at least 85% identical to SEQ ID NO: 6; a forward loop primer (LF) sequence that is at least 85% identical to SEQ ID NO: 7; and a backward loop primer (LB) sequence that is at least 85% identical to SEQ ID NO: 8; and including the primer set in a reverse transcription loop-mediated isothermal amplification (RT-LAMP) procedure containing the sample.

In one example of a method of detecting a target pathogen from a Coronaviridae family in a sample, the target pathogen can be a coronavirus selected from: Severe Acute Respiratory Syndrome (SARS)-CoV (SARS-CoV), Severe Acute Respiratory Syndrome (SARS)-CoV 2 (SARS-CoV-2), Middle East Respiratory Syndrome (MERS)-CoV (MERS-CoV), SARS-CoV hCoV-HKU1, hCoV-0C43, hCoV-NL63, and hCoV-229E.

In another example of a method of detecting a target pathogen from a Coronaviridae family in a sample, the sample can be from a human subject.

In another example of a method of detecting a target pathogen from a Coronaviridae family in a sample, the target pathogen can be Severe Acute Respiratory Syndrome (SARS)-CoV 2 (SARS-CoV-2).

In another example of a method of detecting a target pathogen from a Coronaviridae family in a sample, the method can further comprise observing an output test indicator of the RT-LAMP process indicating the presence or absence of the target pathogen.

In another example of a method of detecting a target pathogen from a Coronaviridae family in a sample, the output test indicator is a color indicator.

In one example there is provided a primer set for reverse transcription loop-mediated isothermal amplification (RT-LAMP) analysis which comprises: a forward inner primer (FIP) sequence that is at least 85% identical to a combination of SEQ ID NO: 11 and SEQ ID NO: 12; a backward inner primer (BIP) sequence that is at least 85% identical to a combination of seq ID NO: 13 and SEQ ID NO: 14. a forward outer primer (F3) sequence that is at least 85% identical to SEQ ID NO: 15; a backward outer primer (B3) sequence that is at least 85% identical to SEQ ID NO: 16; a forward loop primer (LF) sequence that is at least 85% identical to SEQ ID NO: 17; and a backward loop primer (LB) sequence that is at least 85% identical to SEQ ID NO: 18.

In one example of a primer set for reverse transcription loop-mediated isothermal amplification (RT-LAMP) analysis, the primer set can comprise the FIP sequence can be 100% identical to a combination of SEQ ID NO: 11 and SEQ ID NO: 12, which is equivalent to SEQ ID NO: 19.

In another example of a primer set for reverse transcription loop-mediated isothermal amplification (RT-LAMP) analysis, the FIP sequence can further comprise a linking sequence joining SEQ ID NO: 11 and SEQ ID NO: 12, wherein the linking sequence is selected from Table 11.

In another example of a primer set for reverse transcription loop-mediated isothermal amplification (RT-LAMP) analysis, the BIP sequence can be 100% identical to a combination of SEQ ID NO: 13 and SEQ ID NO: 14, which is equivalent to SEQ ID NO: 20.

In another example of a primer set for reverse transcription loop-mediated isothermal amplification (RT-LAMP) analysis, the BIP sequence can further comprise a linking sequence joining SEQ ID NO: 13 and SEQ ID NO: 14, wherein the linking sequence is selected from Table 11.

In one example there is provided, a primer set for reverse transcription loop-mediated isothermal amplification (RT-LAMP) analysis that can comprise: a forward inner primer (FIP) sequence that is at least 85% identical to a combination of SEQ ID NO: 21 and SEQ ID NO: 22;

a backward inner primer (BIP) sequence that is at least 85% identical to a combination of seq ID NO: 23 and SEQ ID NO: 24. a forward outer primer (F3) sequence that is at least 85% identical to SEQ ID NO: 25; a backward outer primer (B3) sequence that is at least 85% identical to SEQ ID NO: 26; a forward loop primer (LF) sequence that is at least 85% identical to SEQ ID NO: 27; and a backward loop primer (LB) sequence that is at least 85% identical to SEQ ID NO: 28.

In one example of a primer set for reverse transcription loop-mediated isothermal amplification (RT-LAMP) analysis, the FIP sequence can be 100% identical to a combination of SEQ ID NO: 21 and SEQ ID NO: 22 which is equivalent to SEQ ID NO: 29.

In another example of a primer set for reverse transcription loop-mediated isothermal amplification (RT-LAMP) analysis, the FIP sequence can further comprise a linking sequence joining SEQ ID NO: 21 and SEQ ID NO: 22, wherein the linking sequence is selected from Table 11.

In another example of a primer set for reverse transcription loop-mediated isothermal amplification (RT-LAMP) analysis, the BIP sequence can be 100% identical to a combination of SEQ ID NO: 23 and SEQ ID NO: 24, which is equivalent to SEQ ID NO: 30.

In another example of a primer set for reverse transcription loop-mediated isothermal amplification (RT-LAMP) analysis, the BIP sequence can further comprise a linking sequence joining SEQ ID NO: 23 and SEQ ID NO: 24, wherein the linking sequence is selected from Table 11.

In one example there is provided, a primer set for reverse transcription loop-mediated isothermal amplification (RT-LAMP) analysis that can comprise: a forward inner primer (FIP) sequence that is at least 85% identical to a combination of SEQ ID NO: 31 and SEQ ID NO: 32; a backward inner primer (BIP) sequence that is at least 85% identical to a combination of seq ID NO: 33 and SEQ ID NO: 34. a forward outer primer (F3) sequence that is at least 85% identical to SEQ ID NO: 35; a backward outer primer (B3) sequence that is at least 85% identical to SEQ ID NO: 36; a forward loop primer (LF) sequence that is at least 85% identical to SEQ ID NO: 37; and a backward loop primer (LB) sequence that is at least 85% identical to SEQ ID NO: 38.

In one example of a primer set for reverse transcription loop-mediated isothermal amplification (RT-LAMP) analysis, the FIP sequence can be 100% identical to a combination of SEQ ID NO: 31 and SEQ ID NO: 32, which is equivalent to SEQ ID NO: 39.

In another example of a primer set for reverse transcription loop-mediated isothermal amplification (RT-LAMP) analysis, the FIP sequence can further comprise a linking sequence joining SEQ ID NO: 31 and SEQ ID NO: 32, wherein the linking sequence is selected from Table 11.

In another example of a primer set for reverse transcription loop-mediated isothermal amplification (RT-LAMP) analysis, the BIP sequence can be 100% identical to a combination of SEQ ID NO: 33 and SEQ ID NO: 34 which is equivalent to SEQ ID NO: 40.

In another example of a primer set for reverse transcription loop-mediated isothermal amplification (RT-LAMP) analysis, the BIP sequence can further comprise a linking sequence joining SEQ ID NO: 33 and SEQ ID NO: 34, wherein the linking sequence is selected from Table 11.

In yet another example there is provided, a primer set for reverse transcription loop-mediated isothermal amplification (RT-LAMP) analysis that can comprise: a forward inner primer (FIP) sequence that is at least 85% identical to a combination of SEQ ID NO: 41 and SEQ ID NO: 42; a backward inner primer (BIP) sequence that is at least 85% identical to a combination of seq ID NO: 43 and SEQ ID NO: 44. a forward outer primer (F3) sequence that is at least 85% identical to SEQ ID NO: 45; a backward outer primer (B3) sequence that is at least 85% identical to SEQ ID NO: 46; a forward loop primer (LF) sequence that is at least 85% identical to SEQ ID NO: 47; and a backward loop primer (LB) sequence that is at least 85% identical to SEQ ID NO: 48.

In one example of a primer set for reverse transcription loop-mediated isothermal amplification (RT-LAMP) analysis, the FIP sequence can be 100% identical to a combination of SEQ ID NO: 41 and SEQ ID NO: 42 which is equivalent to SEQ ID NO: 49.

In another example of a primer set for reverse transcription loop-mediated isothermal amplification (RT-LAMP) analysis, the FIP sequence can further comprise a linking sequence joining SEQ ID NO: 41 and SEQ ID NO: 42, wherein the linking sequence is selected from Table 11.

In another example of a primer set for reverse transcription loop-mediated isothermal amplification (RT-LAMP) analysis, the BIP sequence can be 100% identical to a combination of SEQ ID NO: 43 and SEQ ID NO: 44 which is equivalent to SEQ ID NO: 50.

In another example of a primer set for reverse transcription loop-mediated isothermal amplification (RT-LAMP) analysis, the BIP sequence can further comprise a linking sequence joining SEQ ID NO: 43 and SEQ ID NO: 44, wherein the linking sequence is selected from Table 11.

In one example there is provided, a primer set for reverse transcription loop-mediated isothermal amplification (RT-LAMP) analysis which can comprise: a forward inner primer (FIP) sequence that is at least 85% identical to a combination of SEQ ID NO: 51 and SEQ ID NO: 52; a backward inner primer (BIP) sequence that is at least 85% identical to a combination of seq ID NO: 53 and SEQ ID NO: 54. a forward outer primer (F3) sequence that is at least 85% identical to SEQ ID NO: 55; a backward outer primer (B3) sequence that is at least 85% identical to SEQ ID NO: 56; a forward loop primer (LF) sequence that is at least 85% identical to SEQ ID NO: 57; and a backward loop primer (LB) sequence that is at least 85% identical to SEQ ID NO: 58.

In one aspect, the FIP sequence can be 100% identical to a combination of SEQ ID NO: 51 and SEQ ID NO: 52 which is equivalent to SEQ ID NO: 59.

In another example of a primer set for reverse transcription loop-mediated isothermal amplification (RT-LAMP) analysis, the FIP sequence can further comprise a linking sequence joining SEQ ID NO: 51 and SEQ ID NO: 52, wherein the linking sequence is selected from Table 11.

In another example of a primer set for reverse transcription loop-mediated isothermal amplification (RT-LAMP) analysis, the BIP sequence can be 100% identical to a combination of SEQ ID NO: 53 and SEQ ID NO: 54, which is equivalent to SEQ ID NO: 60.

In another example of a primer set for reverse transcription loop-mediated isothermal amplification (RT-LAMP) analysis, the BIP sequence can further comprise a linking sequence joining SEQ ID NO: 53 and SEQ ID NO: 54, wherein the linking sequence is selected from Table 11.

In one example there is provided, a primer set for reverse transcription loop-mediated isothermal amplification (RT-LAMP) analysis which can comprise: a forward inner primer (FIP) sequence that is at least 85% identical to a combination of SEQ ID NO: 61 and SEQ ID NO: 62; a backward inner primer (BIP) sequence that is at least 85% identical to a combination of seq ID NO: 63 and SEQ ID NO: 64. a forward outer primer (F3) sequence that is at least 85% identical to SEQ ID NO: 65; a backward outer primer (B3) sequence that is at least 85% identical to SEQ ID NO: 66; a forward loop primer (LF) sequence that is at least 85% identical to SEQ ID NO: 67; and a backward loop primer (LB) sequence that is at least 85% identical to SEQ ID NO: 68.

In one example of a primer set for reverse transcription loop-mediated isothermal amplification (RT-LAMP) analysis, the FIP sequence can be 100% identical to a combination of SEQ ID NO: 61 and SEQ ID NO: 62 which is equivalent to SEQ ID NO: 69.

In another example of a primer set for reverse transcription loop-mediated isothermal amplification (RT-LAMP) analysis, the FIP sequence can further comprise a linking sequence joining SEQ ID NO: 61 and SEQ ID NO: 62, wherein the linking sequence is selected from Table 11.

In another example of a primer set for reverse transcription loop-mediated isothermal amplification (RT-LAMP) analysis, the BIP sequence can be 100% identical to a combination of SEQ ID NO: 63 and SEQ ID NO: 64, which is equivalent to SEQ ID NO: 70.

In another example of a primer set for reverse transcription loop-mediated isothermal amplification (RT-LAMP) analysis, the BIP sequence can further comprise a linking sequence joining SEQ ID NO: 63 and SEQ ID NO: 64, wherein the linking sequence is selected from Table 11.

It should be understood that the above-described methods are only illustrative of some embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that variations including, may be made without departing from the principles and concepts set forth herein.

What is claimed is:

1. An isolated complementary DNA (cDNA) of a nucleic acid molecule, comprising:
a nucleotide sequence that is at least 85% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 or a combination thereof.

2. The isolated cDNA of the nucleic acid molecule of claim 1, wherein the nucleotide sequence is at least 85% identical to SEQ ID NO: 9 or SEQ ID 10.

3. The isolated cDNA of the nucleic acid molecule of claim 1, wherein the nucleotide sequence comprises a linking sequence selected from Table 11 joining SEQ ID NO: 1 to SEQ ID NO: 2, or SEQ ID NO: 3 to SEQ ID NO: 4.

4. The isolated cDNA of the nucleic acid molecule of claim 1, wherein the guanine and cytosine (GC) content of the nucleotide sequence is 50% or less.

5. The isolated cDNA of the nucleic acid molecule of claim 1, wherein an end stability of the nucleotide sequence is less than −3.5 kcal/mol.

6. The isolated cDNA of the nucleic acid molecule of claim 1, wherein the nucleotide sequence has a melting temperature of from about 40° C. to about 62° C.

7. The isolated cDNA of the nucleic acid molecule of claim 1, wherein the nucleotide sequence has a minimum primer dimerization energy of less than −3 kcal/mol.

8. The isolated cDNA of the nucleic acid molecule of claim 1, wherein the nucleotide sequence is between 90% and 100% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 or a combination thereof.

9. A primer set for reverse transcription loop-mediated isothermal amplification (RT-LAMP) analysis, comprising:
a forward inner primer (FIP) sequence that is at least 85% identical to a combination of SEQ ID NO: 1 and SEQ ID NO: 2;
a backward inner primer (BIP) sequence that is at least 85% identical to a combination of seq ID NO: 3 and SEQ ID NO: 4.
a forward outer primer (F3) sequence that is at least 85% identical to SEQ ID NO: 5;
a backward outer primer (B3) sequence that is at least 85% identical to SEQ ID NO: 6;
a forward loop primer (LF) sequence that is at least 85% identical to SEQ ID NO: 7; and
a backward loop primer (LB) sequence that is at least 85% identical to SEQ ID NO: 8.

10. The primer set of claim 9, wherein the FIP sequence further comprises a linking sequence from Table 11 joining:
SEQ ID NO: 1 and SEQ ID NO: 2; or
SEQ ID NO: 3 and SEQ ID NO: 4.

11. The primer set of claim 9, wherein the guanine and cytosine (GC) content of the FIP, the BIP, the F3, the B3, the LF, the LB, or a combination thereof is 50% or less.

12. The primer set of claim 9, wherein an end stability of the FIP, the BIP, the F3, the B3, the LF, the LB, or a combination thereof is less than −2.5 kcal/mol.

13. The primer set of claim 9, wherein the FIP, the BIP, the F3, the B3, the LF, the LB, or a combination thereof has a melting temperature of from about 40° C. to about 62° C.

14. The primer set of claim 9, wherein the FIP, the BIP, the F3, the B3, the LF, the LB, or a combination thereof has a minimum primer dimerization energy of less than −3.0 kcal/mol.

15. The primer set of claim 9, wherein:
the FIP sequence is from 90% to 100% identical to a combination of SEQ ID NO: 1 and SEQ ID NO: 2;
the BIP sequence is from 90% to 100% identical to a combination of seq ID NO: 3 and SEQ ID NO: 4;
the F3 sequence is from 90% to 100% identical to SEQ ID NO: 5;
the B3 sequence is from 90% to 100% identical to SEQ ID NO: 6;
the LF sequence is from 90% to 100% identical to SEQ ID NO: 7; and
the LB sequence is from 90% to 100% identical to SEQ ID NO: 8.

16. A method of detecting a target pathogen from a Coronaviridae family in a subject, comprising:
providing a primer set comprising:
a forward inner primer (FIP) sequence that is at least 85% identical to a combination of SEQ ID NO: 1 and SEQ ID NO: 2;
a backward inner primer (BIP) sequence that is at least 85% identical to a combination of seq ID NO: 3 and SEQ ID NO: 4.
a forward outer primer (F3) sequence that is at least 85% identical to SEQ ID NO: 5;
a backward outer primer (B3) sequence that is at least 85% identical to SEQ ID NO: 6;
a forward loop primer (LF) sequence that is at least 85% identical to SEQ ID NO: 7;
a backward loop primer (LB) sequence that is at least 85% identical to SEQ ID NO: 8; and
including the primer set in a reverse transcription loop-mediated isothermal amplification (RT-LAMP) procedure containing a biological sample from the subject.

17. The method of claim 16, wherein the target pathogen is a human coronavirus selected from: Severe Acute Respiratory Syndrome (SARS)-CoV (SARS-CoV), Severe Acute Respiratory Syndrome (SARS)-CoV 2 (SARS-CoV-2), Middle East Respiratory Syndrome (MERS)-CoV (MERS-CoV), SARS-CoV hCoV-HKU1, hCoV-0C43, hCoV-NL63, and hCoV-229E.

18. The method of claim 16, wherein the subject is a human subject.

19. The method of claim 16, wherein the target pathogen is Severe Acute Respiratory Syndrome (SARS)-CoV 2 (SARS-CoV-2).

20. The method of claim 16, further comprising observing an output test indicator of the RT-LAMP process indicating the presence or absence of the target pathogen.

21. The method of claim 20, wherein the output test indicator is a color indicator.
